(19) United States
(12) Patent Application Publication
(10) Pub. No.: US 2011/0118116 A1
(43) Pub. Date: May 19, 2011

(54) Azolin-2-ylamino Compounds for Combating Animal Pests

(76) Inventors: Ronan Le Vezouet, Mannheim (DE); Markus Kordes, Bobenheim-Roxheim (DE); Ralph Paulini, Bad Durkheim (DE); Deborah L. Culbertson, Fuquay Varina, NC (US)

(21) Appl. No.: 13/003,927

(22) PCT Filed: Jul. 14, 2009
(86) PCT No.: PCT/EP2009/058981

§ 371 Date: Jan. 13, 2011

Related U.S. Application Data
(60) Provisional application No. 61/081,568, filed on Jul. 17, 2008.

(51) Int. Cl. A01N 43/78; C07D 277/42; C07C 335/12; A61K 31/426; A01P 7/04; A01P 7/02; A61P 33/14; A01C 1/06

(52) U.S. Cl. 504/100; 548/190; 514/370; 560/251; 564/28; 548/193

(57) ABSTRACT

The present invention relates to azolin-2-ylamino compounds of formula (I), wherein A is a radical of the formulae (A.1) or (A.2), wherein * denotes the binding site and wherein the indices and variables are as defined in the description. The present invention relates as well as to the precursor compounds thereof, which are both useful for combating animal pests selected from insects, arachnids and nematodes. The invention also relates to methods for controlling and/or combating such animal pests by using these compounds, to methods of protecting crops and plant propargation material, and further also to seeds and agricultural and veterinary composition comprising said compounds.

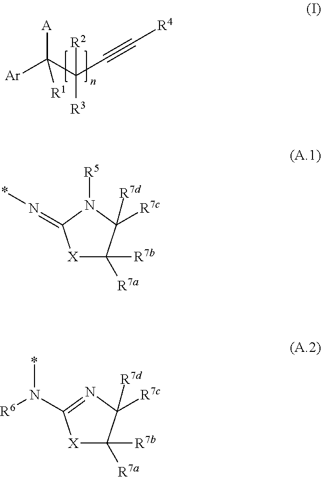

The present invention relates to azolin-2-ylamino compounds as well as to their precursor compounds, which are both useful for combating animal pests. The invention also relates to a method for controlling animal pests by using these compounds, to seed and to an agricultural and veterinary composition comprising said compounds.

Animal pests and in particular arthropods and nematodes destroy growing and harvested crops and attack wooden dwelling and commercial structures, causing large economic loss to the food supply and to property. While a large number of pesticidal agents are known, due to the ability of target pests to develop resistance to said agents, there is an ongoing need for new agents for combating insects, arachnids and nematodes. It is therefore an object of the present invention to provide compounds having a good pesticidal activity and showing a broad activity spectrum against a large number of different animal pests, especially against difficult to control insects, arachnids and nematodes.

Jennings et al. Pesticide Biochemistry and Physiology 30, 1988, p. 190-197 describe several 2-phenylamino oxazolines and 2-benzylamino oxazolines which have insecticidal activity. Insecticicidal active derivates of phenylalkylamines and benzylamines have been described in WO2008/119506 and WO2008/119511. Insecticidal substituted thiourea compounds are disclosed in WO2008/138499. Biosci. Biotech. Biochem. 1992, 56 (7), 1062-1065 discloses phenyl-, benzyl- and phenethyl thiazolines having insecticidal activity. However, these compounds are limited in their activity or with regard to breadth of their activity spectrum. WO 2005/063724 describes 1-(azolin-2-yl)amino-1,2-diphenylethane compounds of the general formulae C.1 and C.2,

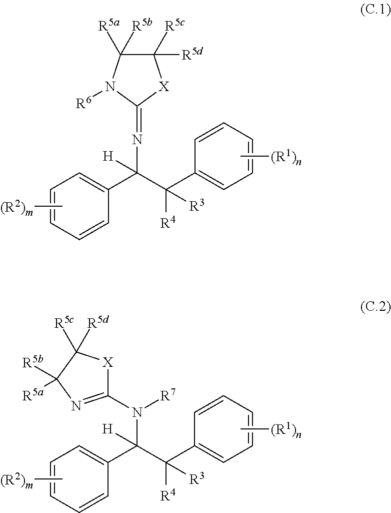

wherein X is sulfur or oxygen, which are useful for combating insects, arachnids and nematodes.

It is an object of the present invention to provide compounds that have a good pesticidal activity, in particular insecticidal activity, and show a broad activity spectrum against a large number of different animal pests, especially against difficult to control insects.

It has been found that these objectives can be achieved by compounds of the formula I below.

Therefore, in a first aspect the invention relates to azolin-2-ylamino compounds of formula (I),

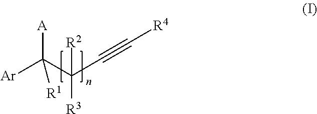

wherein
n is 0, 1, 2 or 3;
Ar is phenyl, which is unsubstituted or carries 1, 2, 3, 4 or 5 substituents R^{Ar}, wherein
   R^{Ar} is selected from halogen, CN, N₃, NO₂, C₁-C₆-alkyl, C₂-C₆-alkenyl and C₂-C₆-alkynyl, wherein each carbon atom in the aforementioned radicals may carry any combination of 1, 2 or 3 substituents, independently of one another selected from halogen, CN, NO₂, NH₂, OH, SH, COOH, C₁-C₆-alkoxy, C₂-C₆-alkenyloxy, C₂-C₆-alkynyloxy, C₁-C₆-haloalkoxy and C₁-C₆-alkylthio, and
   R^{Ar} may further be selected from —C(═O)R^{a}, —C(═S)R^{a}, —C(═NR^{f})R^{a}, —C(═NR^{f})OR^{b}, —C(═NR^{f})NR^{c}R^{d}, —C(═NR^{f})SR^{e}, —C(═O)OR^{b}, —C(═O)NR^{c}R^{d}, —C(═O)SR^{e}, —C(═S)OR^{b}, —C(═S)NR^{c}R^{d}, —C(═S)SR^{e}, —OR^{b}, —O—C(═O)R^{a}, —O—C(═O)OR^{b}, —O—C(═O)—NR^{c}R^{d}, —O—C(═O)SR^{e}, —SR^{e}, —S(═O)R^{e}, —S(═O)₂R^{e}, —S(═O)₂OR^{b}, —S(═O)₂NR^{c}R^{d}, —NR^{c}R^{d}, —NR^{f}—C(═O)R^{a}, —NR^{f}—C(═O)OR^{b}, —NR^{f}—C(═O)NR^{c}R^{d}, —N═CR^{a}R^{a′}, —NR^{f}—NR^{c}R^{d}, —NR^{f}—C(═O)SR^{e} and —NR^{f}—C(═S)NR^{c}R^{d}, wherein R^{a}, R^{a′}, R^{b}, R^{c}, R^{d}, R^{e} and R^{f} independently of one another have one of the meanings given below, and
   R^{Ar} may further be selected from cyclic radicals —Y-Cy or —Y—Ar¹, wherein Ar¹ has one of the meanings given below,
      Y is a single bond, C₁-C₆-alkandiyl, C₁-C₆-alkandiyloxy, —C(═O)—, —C(═S)—, —C(═NR^{f})—, —C(═NR^{f})O—, —C(═NR^{f})S—, —C(═NR^{f})NR^{c}, —C(═O)O—, —C(═O)NR^{c}—, —C(═O)S—, —C(═S)O—, —C(═S)NR^{c}—, —C(═S)S—, —O—, —O—C(═O)—, —O—C(═O)O—, —O—C(═O)—NR^{c}—, —O—C(═O)S—, —S—, —S(═O)—, —S(═O)₂—, —S(═O)₂O—, —S(═O)₂NR^{c}—, —NR^{c}—, —NR^{f}—C(═O)—, —NR^{f}—C(═O)O—, —NR^{f}—C(═O)NR^{c}—, —N═CR^{a}—, —NR^{f}—NR^{c}—, —NR^{f}—C(═O)S—, —NR^{f}—C(═S)NR^{c}—, wherein R^{a}, R^{b}, R^{c} and R^{f} independently of one another have one of the meanings given below,
      Cy is C₃-C₁₂-cycloalkyl or a saturated or partially unsaturated 5, 6 or 7-membered heterocycle containing 1, 2, 3 or 4 heteroatoms selected independently from one another from S, O and N as ring members, wherein C₃-C₁₂-cycloalkyl and the heterocycle are each unsubstituted or carry any combination of 1, 2, 3, 4 or 5 substituents, independently of one another selected from halogen, halogen, CN, NO₂, OH, SH, NH₂, COOH, C₁-C₆-alkyl, C₁-C₆-haloalkyl, C₁-C₆-alkoxy, C₂-C₆-alkenyloxy, C₂-C₆-alkynyloxy, C₁-C₆-haloalkoxy and C₁-C₆-alkylthio,
   and/or two radicals R^{Ar} bound to adjacent carbon atoms of the phenyl ring together with said carbon atoms form a fused benzene, a fused saturated or partially unsaturated 5-, 6- or 7-membered carbocycle or a fused 5-, 6- or 7-membered heterocycle, which contains 1, 2, 3 or 4 heteroatoms selected from O, S and N as ring members, wherein each fused benzene, carbocycle or heterocycle is unsubstituted or carries 1, 2, 3 or 4 substituents R^{Ar} as defined above with the exception that such two radicals R^{Ar} when bound to adjacent carbon atoms of the fused benzene, carbocycle or heterocycle do not form another fused ring system;

A is a radical of the formulae A.1 or A.2,

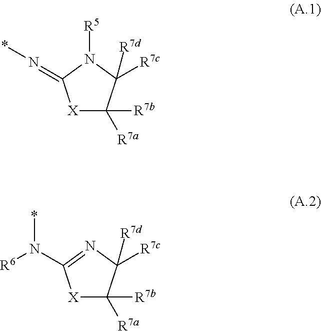

wherein
   indicates the point of attachment to the remaining part of the compound;
X is S, O or N(R⁸);
R^{7a}, R^{7b}, R^{7c} and R^{7d} are selected independently from one another from hydrogen, halogen, C₁-C₆-alkyl, C₁-C₆-alkoxy, C₁-C₆-alkylamino and C₃-C₆-cycloalkyl, wherein
   each carbon atom of the aforementioned radicals may be unsubstituted or carries any combination of 1, 2 or 3 substituents, independently of one another selected from halogen, CN, NO₂, —OR^{b}, —SR^{e}, —NR^{c}R^{d}, C(═O)—R^{a}, C(═O)OR^{b} and Ar², wherein Ar², R^{a}, R^{b}, R^{c}, R^{d} and R^{e} have one of the meanings given below and wherein C₃-C₆-cycloalkyl additionally may carry any combination of 1, 2 or 3 substituents of one another selected from C₁-C₆-alkyl, C₂-C₆-alkenyl, C₂-C₆-alkynyl and C₁-C₆-haloalkyl,
R⁵, R⁶ and R⁸ are selected each independently from one another from hydrogen, CN, NO₂, C₁-C₆-alkyl, C₂-C₆-alkenyl, C₂-C₆-alkynyl, C₃-C₈-cycloalkyl and Ar³, wherein C₁-C₆-alkyl, C₂-C₆-alkenyl, C₂-C₆-alkynyl and C₃-C₈-cycloalkyl are unsubstituted or carry any combination of 1, 2, 3, 4 or 5 substituents, independently of one another selected from halogen, CN, NO₂, —OR^{b}, —NR^{c}R^{d}, —SR^{e}, —C(═O)R^{a}, —C(═O)OR^{b}, saturated or partially unsaturated 5, 6 or 7-membered heterocycles containing 1, 2, 3 or 4 heteroatoms selected independently from one another from S, O and N as ring members, —Ar⁴, —O—Ar⁴, —S—Ar⁴ or —CH₂—Ar⁴, wherein Ar³, Ar⁴, R^{a}, R^{b}, R^{c}, R^{d}, R^{e} and R^{f} have one of the meanings given below, and wherein C₃-C₈-cycloalkyl and the heterocycles additionally may carry any combination of 1, 2 or 3 or substituents, independently of one another selected from C₁-C₆-alkyl, C₁-C₆-haloalkyl, C₂-C₆-alkenyl and C₂-C₆-alkynyl, and
R⁵, R⁶ and R⁸ may further be selected from -E-C(═O)R^{a}, -E-C(═S)R^{a}, -E-C(═NR^{f})R^{a}, -E-C(═NR^{f})OR^{b}, -E-C(═NR^{f})NR^{c}R^{d}, -E-C(═NR^{f})SR^{e}, -E-C(═O)OR^{b}, -E-C(═O)NR^{c}R^{d}, -E-C(═O)SR^{e}, -E-C(═S)OR^{b}, -E-C(═S)NR^{c}R^{d}, -E-C(═S)SR^{e}, -E-OR^{b}, -E-O—C(═O)R^{a}, -E-O—C(═O)OR^{b}, -E-O—C(═O)—NR^{c}R^{d}, -E-O—C(═O)SR^{e}, -E-SR^{e}, -E-NR^{c}R^{d}, -E-NR^{f}—C(═O)R^{a}, -E-NR^{f}—C(═O)OR^{b}, -E-NR^{f}—C(═O)NR^{c}R^{d}, -E-N═CR^{a}R^{a′}, -E-NR^{f}—NR^{c}R^{d}, -E-NR^{f}—C(═O)SR^{e}, -E-NR^{f}—C(═S)NR^{c}R^{d}, -E-S(═O)R^{e}, -E-S(═O)₂R^{e}, —S(═O)₂OR^{b}, —S(═O)₂NR^{c}R^{d}, —P(═O)R^{g}R^{d} and P(═S)R^{g}R^{h}, wherein
   E is a single bond or linear or branched C₁-C₄-alkanediyl, and
   R^{a}, R^{a′}, R^{b}, R^{c}, R^{d}, R^{e}, R^{f}, R^{g} and R^{h} independently of one another have one of the meanings given below;
R¹ is selected from hydrogen, halogen, C₁-C₆-alkyl, C₂-C₆-alkenyl, C₂-C₆-alkynyl, C₃-C₆-cycloalkyl, phenyl and benzyl, wherein each carbon atom of the aforementioned radicals is unsubstituted or carries any combination of 1, 2 or 3 substituents selected independently of one another from halogen, CN, NO₂, —OR^{b}, NR^{c}R^{d}, —SR^{e}, —C(═O)R^{a} and —C(═O)OR^{b}, wherein R^{a}, R^{b}, R^{c}, R^{d} and R^{e} have one of the meanings given below, and wherein C₃-C₆-cycloalkyl, phenyl and benzyl additionally may carry 1, 2, 3, 4 or 5 substituents selected independently of one another from C₁-C₆-alkyl, C₂-C₆-alkenyl, C₂-C₆-alkynyl and C₁-C₆-haloalkyl;
R² and R³ are selected each independently of one another from hydrogen, halogen, C₁-C₆-alkyl, C₂-C₆-alkenyl, C₂-C₆-alkynyl, C₃-C₆-cycloalkyl, phenyl and benzyl, wherein each carbon atom of the aforementioned radicals is un-substituted or carries any combination of 1, 2 or 3 radicals selected independently of one another from the group consisting of halogen, CN, NO₂, —OR^{b}, NR^{c}R^{d}, —SR^{e} —C(═O)R^{a} and —C(═O)OR^{b}, wherein R^{a}, R^{b}, R^{c}, R^{d} and R^{e} have one of the meanings given above, and wherein C₃-C₁-cycloalkyl, phenyl and benzyl additionally may carry 1, 2, 3, 4 or 5 substituents selected independently of one another from C₁-C₆-alkyl, C₂-C₆-alkenyl, C₂-C₆-alkynyl and C₁-C₆-haloalkyl,
   wherein, when n is 2 or 3, the respective 2 or 3 substituents R² and 2 or 3 substituents R³ of this azolin-2-ylamino compound of formula I are selected independently from one another.
R⁴ is selected from hydrogen, halogen, CN, C₁-C₆-alkyl, C₂-C₆-alkenyl, C₂-C₆-alkynyl, C₃-C₈-cycloalkyl, saturated or partially unsaturated 5, 6 or 7-membered heterocycle containing 1, 2, 3 or 4 heteroatoms selected independently from one another from O, S and N as ring members, and Ar⁵, wherein C₁-C₆-alkyl, C₂-C₆-alkenyl, C₂-C₆-alkynyl, C₃-C₈-cycloalkyl and the heterocycle are unsubstituted or carry any combination of 1, 2, 3, 4 or 5 substituents, independently of one another selected from halogen, CN, NO₂, —OR^{b}, —NR^{c}R^{d}, —SR^{e}, —C(═O)R^{a}, —C(═O)OR^{b}, Ar⁶, —CH₂—Ar⁶, —O—Ar⁶, and —S—Ar⁶, wherein R^{b}, R^{c}, R^{d} R^{e}, Ar⁵ and Ar⁶ have one of the meanings given below, and wherein C₃-C₈-cycloalkyl and the heterocycle additionally may carry 1, 2, 3, 4 or 5 substituents selected independently of one another from C₁-C₆-alkyl, C₂-C₆-alkenyl, C₂-C₆-alkynyl and C₁-C₆-haloalkyl, and
   R⁴ may further be selected from —SiR^{a}R^{b}R^{c}, —Si(OR^{a})R^{b}R^{c}, —Si(OR^{a})(OR^{b})R^{c}, —Si(OR^{a})(OR^{b})(OR^{c}), —Si(—NR^{a}R^{b})R^{c}R^{d}, —C(═O)R^{a}, —C(═S)R^{a}, —C(═NR^{f})R^{a}, —C(═NR^{f})OR^{b}, —C(═NR^{f})NR^{c}R^{d}, —C(═NR^{f})SR^{e}, —C(═O)OR^{b}, —C(═O)NR^{c}R^{e}, —C(═S)OR^{b}, —C(═S)NR^{c}R^{d}, —OR^{b}, —O—C(═O)R^{a}, —O—C(═O)OR^{b}, —O—C(═O)—NR^{c}R^{d}, —SR^{e}, —S(═O)R^{e}, —S(═O)₂R^{e}, —S(═O)₂NR^{c}R^{d}, —S(═N—R^{a})R^{b}, —S(═O)(═N—R^{a})R^{b}, —NR^{c}R^{d}, —NR^{f}—C(═O)R^{a}, —NR^{f}—C(═O)OR^{b}, —NR^{f}—C(═O)NR^{c}R^{d}, —N═CR^{a}R^{a′}, —NR^{f}—NR^{c}R^{d}, —NR^{f}—C(═O)SR^{e} and —NR^{f}—C(═S)NR^{c}R^{d};
   wherein R^{a}, R^{a′}, R^{b}, R^{c}, R^{d}, R^{e}, R^{f}, R^{g} and R^{h} are independently of one another selected from hydrogen, C₁-C₆-alkyl, C₂-C₆-alkenyl, C₂-C₆-alkynyl, C₃-C₈-cycloalkyl, saturated or partially unsaturated 5, 6 or 7-membered heterocycles containing 1, 2, 3 or 4 heteroatoms selected independently from one another from O, S, N as ring members, and Ar⁷,
   wherein each carbon atom in the aforementioned radicals may carry any combination of 1, 2 or 3 substituents, independently of one another selected from halogen, CN, NO₂, OH, C₁-C₆-alkoxy, C₁-C₆-haloalkoxy, C₁-C₆-alkylthio, C₁-C₆-haloalkylthio, C₁-C₆-alkylcarbonyl, C₁-C₆-haloalkylcarbonyl, C₁-C₆-alkoxycarbonyl, C₁-C₆-haloalkoxycarbonyl, C₁-C₆-alkylamino, di(C₁-C₆-alkyl)amino, C₃-C₆-cycloalkyl, C₃-C₆-halocycloalkyl, Ar⁸, —CH₂—Ar⁸, —O—Ar⁸ and —S—Ar⁸, wherein Ar⁷ and Ar⁸ have one of the meanings given below, and wherein C₃-C₈-cycloalkyl and the heterocycles additionally may carry 1, 2, 3, 4 or 5 substituents selected independently of one another from C₁-C₆-alkyl, C₂-C₆-alkenyl, C₂-C₆-alkynyl and C₁-C₆-haloalkyl; and
   Ar¹, Ar², Ar³, A⁴, Ar⁵, Ar⁶, Ar⁷, and Ar⁸ are independently of one another selected from phenyl, naphthyl and mono- or bicyclic 5- to 10-membered heteroaryl, which contains 1, 2, 3 or 4 heteroatoms selected from O, S and N as ring members, wherein phenyl, naphtyl and hetaryl are unsubstituted or carry any combination of 1 to 5 substituents, independently of one another selected from halogen, CN, NO₂, OH, C₁-C₆-alkyl, C₂-C₆-alkenyl, C₂-C₆-alkynyl, C₁-C₆-haloalkyl, C₁-C₆-alkoxy, C₁-C₆-haloalkoxy, C₁-C₆-alkylthio, C₁-C₆-haloalkylthio, C₁-C₆-alkylcarbonyl, C₁-C₆-haloalkylcarbonyl, C₁-C₆-alkoxycarbonyl, C₁-C₆-haloalkoxycarbonyl, C₁-C₆-alkylamino, di(C₁-C₆-alkyl)amino, C₃-C₆-cycloalkyl, C₃-C₆-halocycloalkyl, unsubstituted phenyl and phenyl carrying 1, 2, 3, 4 or 5 substituents selected from halogen, C₁-C₆-alkyl, C₁-C₆-haloalkyl, C₁-C₆-alkoxy and C₁-C₆-haloalkoxy;

or the enantiomers, diastereomers or agriculturally or veterinarily acceptable salts thereof.

The invention relates also to the precursors of compounds of formula (I), namely compounds of general formula (II),

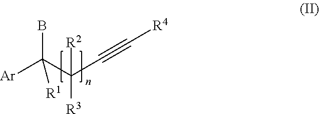

wherein n, Ar, R¹, R², R³, and R⁴ independently from one another have one of the meanings given for the compounds of formula I, and wherein
B is a radical

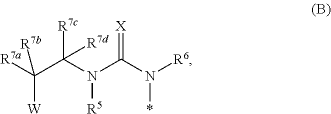

wherein
* indicates the point of attachment to the remaining part of the compound, R⁵, R⁶, R^{7a}, R^{7b}, R^{7c}, R^{7d} and X have the meanings as defined in claim 1 for the compounds of formula I,
   and
   W is halogen or —OR⁹, wherein
   R⁹ is hydrogen or —C(═O)—Rⁱ, wherein
   Rⁱ is selected from hydrogen, C₁-C₆-alkyl, C₂-C₆-alkenyl, C₂-C₆-alkynyl, C₃-C₈-cycloalkyl, saturated or partially unsaturated 5, 6 or 7-membered heterocycles containing 1, 2, 3 or 4 heteroatoms selected independently from one another from O, S and N as ring members, and Ar⁹, wherein
      C₁-C₆-alkyl, C₂-C₆-alkenyl, C₂-C₆-alkynyl, C₃-C₈-cycloalkyl and the heterocycles are unsubstituted or carry any combination of 1, 2, 3, 4 or 5 substituents, independently of one another selected from halogen, CN, NO₂, OH, C₁-C₆-alkoxy, C₁-C₆-haloalkoxy, C₁-C₆-alkylthio, C₁-C₆-haloalkylthio, C₁-C₆-alkylcarbonyl, C₁-C₆-haloalkylcarbonyl, C₁-C₆-alkoxycarbonyl, C₁-C₆-haloalkoxycarbonyl, C₁-C₆-alkylamino, di(C₁-C₆-alkyl)amino, C₃-C₆-cycloalkyl, C₃-C₆-halocycloalkyl, Ar¹⁰, —CH₂—Ar¹⁰, —O—Ar¹⁰ and —S—Ar¹⁰, and wherein
      C₃-C₈-cycloalkyl and the heterocycles additionally may carry 1, 2, 3, 4 or 5 substituents selected independently of one another from C₁-C₆-alkyl, C₂-C₆-alkenyl, C₂-C₆-alkynyl and C₁-C₆-haloalkyl; and wherein
         Ar⁹ and Ar¹⁰ are independently of one another selected from phenyl, naphthyl and mono- or bicyclic 5- to 10-membered heteroaryl, which contains 1, 2, 3 or 4 heteroatoms selected from O, S and N as ring members, wherein phenyl, naphtyl and hetaryl are unsubstituted or carry any combination of 1 to 5 substituents, independently of one another selected from halogen, CN, NO₂, OH, C₁-C₆-alkyl, C₂-C₆-alkenyl, C₂-C₆-alkynyl, C₁-C₆-haloalkyl, C₁-C₆-alkoxy, C₁-C₆-haloalkoxy, C₁-C₆-alkylthio, C₁-C₆-haloalkylthio, C₁-C₆-alkylcarbonyl, C₁-C₆-haloalkylcarbonyl, C₁-C₆-alkoxycarbonyl, C₁-C₆-haloalkoxycarbonyl, C₁-C₆-alkylamino, di(C₁-C₆-alkyl)amino, C₃-C₆-cycloalkyl, C₃-C₆-halocycloalkyl, unsubstituted phenyl and phenyl carrying 1, 2, 3, 4 or 5 substituent selected from halogen, C₁-C₆-alkyl, C₁-C₆-haloalkyl, C₁-C₆-alkoxy and C₁-C₆-haloalkoxy,
         or the enantiomers, diastereomers or agriculturally or veterinarily acceptable salts thereof.

Moreover, the present invention relates to and includes the following embodiments:
agricultural and veterinary compositions comprising an amount of at least one compound of the formula I or II, or an enantiomer, diasteromer or salt thereof;
the use of a compound of formula I or II, or an enantiomer, diasteromer or salt thereof for combating or controlling animal pests;
a method of combating animal pests which comprises contacting the animal pests, their habit, breeding ground, food supply, plant, seed, soil, area, material or environment in which the animal pests are growing or may grow, or the materials, plants, seeds, soils, surfaces or spaces to be protected from animal attack or infestation with a pesticidally effective amount of at least one compound of the formula I or II, or an enantiomer, diasteromer or salt thereof;
a method for protecting crops from attack or infestation by animal pests, which comprises contacting a crop with a pesticidally effective amount of at least one compound of the formula I or II, or an enantiomer, diasteromer or salt thereof;
a method for the protection of plant propargation material, especially seeds, from soil insects and of the seedlings' roots and shoots from soil and foliar insects comprising contacting the plant propargation material, especially the seeds, before sowing and/or after pregermination with at least one compound of the formula I or II, or the enantiomers, diastereomers or salts thereof;
seeds comprising a compound of the formula I or II, or an enantiomer, diasteromer or salt thereof;
the use of compounds of formula I or II, or the enantiomers, diastereomers or veterinary acceptable salts thereof for combating parasites in and on animals;
a method for treating, controlling, preventing or protecting animals against infestation or infection by parasites which comprises orally, topically or parenterally administering or applying to the animals a parasiticidally effective amount of an compound of formula I or II, or the enantiomers, diastereomers and/or veterinary acceptable salt thereof;
a process for the preparation of a composition for treating, controlling, preventing or protecting animals against infestation or infection by parasites which comprises a parasiticidally effective amount of an compound of formula I or II, or the enantiomers, diastereomers and/or veterinary acceptable salt thereof.

In the compounds of formulae I and II, the carbon atom which carries the radical Ar creates a center of chirality. Thus, the compounds of formulae I and II may be present in the form of different enantiomers or if another center of chirality is present, e.g. in any of the radicals R² to R⁴, it may exist in the form of diastereomers. In case that A is a radical of the formula A.1, the compound I may also exist as a cis- or trans-isomer with respect to the N═C axis. The present invention relates to every possible stereoisomer of the compounds of formulae I and II, i.e. to single enantiomers or diastereomers, as well as to mixtures thereof.

Salts of the compounds of formulae I and II are preferably agriculturally and veterinarily acceptable salts. They can be formed in a customary method, e.g. by reacting the compound with an acid of the anion in question if the compound of formulae I or II has a basic functionality or by reacting an acidic compound of formulae I or II with a suitable base.

Suitable agriculturally useful salts are especially the salts of those cations or the acid addition salts of those acids whose cations and anions, respectively, do not have any adverse effect on the action of the compounds according to the present invention. Suitable cations are in particular the ions of the alkali metals, preferably lithium, sodium and potassium, of the alkaline earth metals, preferably calcium, magnesium and barium, and of the transition metals, preferably manganese, copper, zinc and iron, and also ammonium (NH⁴⁺) and substituted ammonium in which one to four of the hydrogen atoms are replaced by C₁-C₄-alkyl, C₁-C₄-hydroxyalkyl, C₁-C₄-alkoxy, C₁-C₄-alkoxy-C₁-C₄-alkyl, hydroxy-C₁-C₄-alkoxy-C₁-C₄-alkyl, phenyl or benzyl. Examples of substituted ammonium ions comprise methylammonium, isopropylammonium, dimethylammonium, diisopropylammonium, trimethylammonium, tetramethylammonium, tetraethylammonium, tetrabutylammonium, 2-hydroxyethylammonium, 2-(2-hydroxyethoxy) thylammonium, bis(2-hydroxyethyl)ammonium, benzyltrimethylammonium and benzyl-triethylammonium, furthermore phosphonium ions, sulfonium ions, preferably tri(C₁-C₄-alkyl)sulfonium, and sulfoxonium ions, preferably tri(C₁-C₄-alkyl)sulfoxonium.

Anions of useful acid addition salts are primarily chloride, bromide, fluoride, hydrogen sulfate, sulfate, dihydrogen phosphate, hydrogen phosphate, phosphate, nitrate, hydrogen carbonate, carbonate, hexafluorosilicate, hexafluorophosphate, benzoate, and the anions of C₁-C₄-alkanoic acids, preferably formate, acetate, propionate and butyrate. They can be formed by reacting a compound of formulae I or II with an acid of the corresponding anion, preferably of hydrochloric acid, hydrobromic acid, sulfuric acid, phosphoric acid or nitric acid.

By the term “veterinarily acceptable salts” is meant salts of those cations or anions which are known and accepted in the art for the formation of salts for veterinary use. Suitable acid addition salts, e.g. formed by compounds of formulae I or II containing a basic nitrogen atom, e.g. an amino group, include salts with inorganic acids, for example hydrochlorids, sulphates, phosphates, and nitrates and salts of organic acids for example acetic acid, maleic acid, dimaleic acid, fumaric acid, difumaric acid, methane sulfenic acid, methane sulfonic acid, and succinic acid.

The organic moieties mentioned in the above definitions of the variables are—like the term halogen—collective terms for individual listings of the individual group members. The prefix Cₙ-Cₘ indicates in each case the possible number of carbon atoms in the group.

The term halogen denotes in each case fluorine, bromine, chlorine or iodine, in particular fluorine, chlorine or bromine.

### Examples of Other Meanings are:

The term “C₁-C₆-alkyl” as used herein and in the alkyl moieties of C₁-C₆-alkoxy, C₁-C₆-alkylamino, di(C₁-C₆-alkyl)amino, C₁-C₆-alkylthio, C₁-C₆-alkylcarbonyl, C₁-C₆-alkoxycarbonyl, C₁-C₆-alkylaminocarbonyl and di(C₁-C₆-alkyl)aminocarbonyl refers to a saturated straight-chain or branched hydrocarbon group having 1 to 6 carbon atoms, especially 1 to 3 carbon groups (═C₁-C₃-alkyl). Examples for C₁-C₃-alkyl are methyl, ethyl, propyl and 1-methylethyl (isopropyl). Examples for C₁-C₆ alkyl further encompass, butyl, 1-methylpropyl (sec-butyl, 2-butyl), 2-methylpropyl (iso-butyl), 1,1-dimethylethyl (tert-butyl), pentyl, 1-methylbutyl, 2-methylbutyl, 3 methylbutyl, 2,2-dimethylpropyl, 1-ethylpropyl, hexyl, 1,1-dimethylpropyl, 1,2 dimethylpropyl, 1-methylpentyl, 2-methylpentyl, 3 methyl pentyl, 4-methylpentyl, 1,1 dimethylbutyl, 1,2-dimethylbutyl, 1,3-dimethylbutyl, 2,2-dimethylbutyl, 2,3 dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2 methylpropyl.

The term “C₁-C₆-haloalkyl” as used herein and in the haloalkyl moieties of C₁-C₆-haloalkoxy, C₁-C₆-haloalkylthio, C₁-C₆-haloalkylcarbonyl and C₁-C₆-haloalkoxycarbonyl refers to a straight-chain or branched saturated alkyl group having 1 to 6 carbon atoms, where some or all of the hydrogen atoms in these groups may be replaced by halogen atoms as mentioned above, for example C₁-C₃-haloalkyl, such as chloromethyl, bromomethyl, dichloromethyl, trichloromethyl, fluoromethyl, difluoromethyl, trifluoromethyl, chloro-fluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2 chloro-2,2-difluoroethyl, 2,2-dichloro-2-fluoroethyl, 2,2,2-trichloroethyl, pentafluoroethyl and the like.

The term “C₁-C₆-alkoxy” as used herein and in the term C₁-C₆-alkoxycarbonyl refers to a straight-chain or branched saturated alkyl group having 1 to 6 carbon atoms, preferably 1 to 3 carbon atoms (═C₁-C₃-alkoxy) which is attached via an oxygen atom. Examples for C₁-C₃-alkoxy include methoxy, ethoxy, OCH₂—C₂H₅ (propoxy) and OCH(CH₃)₂ (isopropoxy). Examples for C₁-C₆-alkoxy further encompass n-butoxy, OCH(CH₃)C₂H₅ (sec-butoxy), OCH₂CH(CH₃)₂ (isobutoxy), OC(CH₃)₃ (tert-butoxy), n-pentoxy, 1 methyl-butoxy, 2-methylbutoxy, 3-methylbutoxy, 1,1-dimethylpropoxy, 1,2-dimethylpropoxy, 2,2-dimethylpropoxy, 1-ethylpropoxy, n-hexoxy, 1 methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2 trimethylpropoxy, 1-ethyl-1-methylpropoxy, 1-ethyl-2-methylpropoxy and the like.

The term “C₁-C₆-haloalkoxy” as used herein and in the term C₁-C₆-haloalkoxycarbonyl refers to a C₁-C₆-alkoxy group as mentioned above wherein the hydrogen atoms are partially or fully substituted by fluorine, chlorine, bromine and/or iodine. Preferred are C₁-C₃-haloalkoxy groups, i.e. C₁-C₃-alkoxy groups as mentioned above wherein the hydrogen atoms are partially or fully substituted by fluorine, chlorine, bromine and/or iodine, for example chloromethoxy, dichloromethoxy, trichloromethoxy, fluoromethoxy, difluoromethoxy, trifluoromethoxy, chlorofluoromethoxy, dichlorofluoromethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, pentafluoroethoxy, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3 trichloropropoxy, 2,2,3,3,3-penta-fluoropropoxy, heptafluoropropoxy, 1-(fluoromethyl)-2-fluoroethoxy, 1-(chloromethyl)-2-chloroethoxy and 1 (bromomethyl)-2-bromoethoxy. Examples for C₁-C₆-haloalkoxy further encompass 4-fluorobutoxy, 4-chlorobutoxy, 4-bromobutoxy, nonafluorobutoxy, 5-fluoro-1-pentoxy, 5-chloro-1-pentoxy, 5 bromo-1-pentoxy, 5-iodo-1-pentoxy, 5,5,5-trichloro-1-pentoxy, undecafluoropentoxy, 6-fluoro-1-hexoxy, 6-chloro-1-hexoxy, 6-bromo-1-hexoxy, 6-iodo-1-hexoxy, 6,6,6 trichloro-1-hexoxy and dodecafluorohexoxy. Particularly preferred are chloromethoxy, fluoromethoxy, difluoromethoxy, trifluoromethoxy, 2-fluoroethoxy, 2 chloroethoxy and 2,2,2-trifluoroethoxy.

The term “C₁-C₆-alkylcarbonyl” as used herein refers to a straight-chain or branched saturated alkyl group having 1 to 6 carbon atoms, preferably 1 to 4 carbon atoms (═C₁-C₄-alkylcarbonyl) bonded via the carbon atom of the carbonyl group at any bond in the alkyl group. Examples for C₁-C₄-alkylcarbonyl include C(═O)CH₃, C(O)C₂H₅, n-propylcarbonyl, 1-methylethylcarbonyl, n-butylcarbonyl, 1-methylpropylcarbonyl, 2-methylpropylcarbonyl and 1,1-dimethylethylcarbonyl. Examples for C₁-C₆-alkyl-carbonyl further encompass n-pentylcarbonyl, 1-methylbutylcarbonyl, 2-methyl-butylcarbonyl, 3-methylbutylcarbonyl, 1,1-dimethylpropylcarbonyl, 1,2-dimethylpropyl-carbonyl, 2,2-dimethylpropylcarbonyl, 1-ethylpropylcarbonyl, n-hexylcarbonyl, 1-methyl-pentylcarbonyl, 2-methylpentylcarbonyl, 3-methylpentylcarbonyl, 4-methylpentyl-carbonyl, 1,1-dimethylbutylcarbonyl, 1,2-dimethylbutylcarbonyl, 1,3-dimethylbutyl-carbonyl, 2,2-dimethylbutylcarbonyl, 2,3-dimethylbutylcarbonyl, 3,3-dimethylbutyl-carbonyl, 1-ethylbutylcarbonyl, 2-ethylbutylcarbonyl, 1,1,2 trimethylpropylcarbonyl, 1,2,2-trimethylpropylcarbonyl, 1-ethyl-1-methylpropylcarbonyl or 1-ethyl-2-methyl-propylcarbonyl and the like.

The term “C₁-C₆-alkoxycarbonyl” as used herein refers to a straight-chain or branched alkoxy group having 1 to 6 carbon atoms, preferably 1 to 4 carbon atoms (═C₁-C₄-alkoxycarbonyl) attached via the carbon atom of the carbonyl group (R—O—C(O)—; R=alkyl). Examples for C₁-C₄-alkoxycarbonyl include —C(O)OCH₃, —C(O)OC₂H₅, —C(O)O—CH₂—C₂H₅, —C(O)OCH(CH₃)₂, n-butoxycarbonyl, —C(O)OCH(CH₃)—C₂H₅, —C(O)—OCH₂CH(CH₃)₂ and C(O)OC(CH₃)₃. Examples for C₁-C₆-alkoxycarbonyl further encompass n-pentoxycarbonyl, 1-methylbutoxycarbonyl, 2-methylbutoxycarbonyl, 3-methylbutoxycarbonyl, 2,2-dimethylpropoxycarbonyl, 1-ethylpropoxycarbonyl, n-hexoxycarbonyl, 1,1-dimethylpropoxycarbonyl, 1,2-dimethylpropoxycarbonyl, 1-methylpentoxycarbonyl, 2-methylpentoxycarbonyl, 3-methylpentoxycarbonyl, 4-methylpentoxycarbonyl, 1,1-dimethylbutoxycarbonyl, 1,2-dimethylbutoxycarbonyl, 1,3-dimethylbutoxycarbonyl, 2,2-dimethylbutoxycarbonyl, 2,3-dimethylbutoxycarbonyl, 3,3-dimethylbutoxycarbonyl, 1-ethylbutoxycarbonyl, 2-ethylbutoxycarbonyl, 1,1,2-trimethylpropoxycarbonyl, 1,2,2-trimethylpropoxycarbonyl, 1-ethyl-1-methylpropoxycarbonyl or 1-ethyl-2-methylpropoxycarbonyl.

The term “C₁-C₆-alkylthio “(C₁-C₆-alkylsulfanyl: C₁-C₆-alkyl-S—)” as used herein refers to a straight-chain or branched saturated alkyl group having 1 to 6 carbon atoms, preferably 1 to 3 carbon atoms (═C₁-C₃-alkylthio) which is attached via a sulfur atom. Examples for C₁-C₃-alkylthio include methylthio, ethylthio, propylthio and 1 methylethylthio. Examples for C₁-C₆-alkylthio further encompass butylthio, 1-methylpropylthio, 2-methylpropylthio and 1,1-dimethylethylthio. n-pentylthio, 1-methylbutylthio, 2-methylbutylthio, 3-methylbutylthio, 2,2-dimethylpropylthio, 1-ethylpropylthio, n-hexylthio, 1,1-dimethylpropylthio, 1,2-dimethylpropylthio, 1-methylpentylthio, 2-methylpentylthio, 3-methylpentylthio, 4-methylpentylthio, 1,1-dimethylbutylthio, 1,2-dimethylbutylthio, 1,3-dimethylbutythio, 2,2-dimethylbutylthio, 2,3-dimethylbutylthio, 3,3-dimethylbutylthio, 1-ethylbutlthio, 2-ethylbutylthio, 1,1,2-trimethylpropylthio, 1,2,2-trimethylpropylthio, 1-ethyl-1-methylpropylthio and 1-ethyl-2-methylpropylthio.

The term “C₁-C₆-haloalkylthio” as used herein refers to a C₁-C₆-alkylthio group as mentioned above wherein the hydrogen atoms are partially or fully substituted by fluorine, chlorine, bromine and/or iodine. Preferred are C₁-C₃-haloalkylthio groups, i.e. C₁-C₃-alkylthio groups as mentioned above wherein the hydrogen atoms are partially or fully substituted by fluorine, chlorine, bromine and/or iodine, for example chloromethylthio, dichloromethylthio, trichloromethylthio, fluoromethylthio, difluoromethylthio, trifluoromethylthio, chlorofluoromethylthio, dichlorofluoromethylthio, chlorodifluoromethylthio, 2-fluoroethylthio, 2-chloroethylthio, 2-bromoethylthio, 2-iodoethylthio, 2,2-difluoroethylthio, 2,2,2-trifluoroethylthio, 2-chloro-2-fluoroethylthio, 2-chloro-2,2-difluoroethylthio, 2,2-dichloro-2-fluoroethylthio, 2,2,2-trichloroethylthio, pentafluoroethyl-thio, 2-fluoropropylthio, 3-fluoropropylthio, 2,2-difluoropropylthio, 2,3-difluoropropylthio, 2-chloropropylthio, 3-chloropropylthio, 2,3-dichloropropylthio, 2-bromopropylthio, 3-bromopropylthio, 3,3,3-trifluoropropylthio, 3,3,3-trichloropropylthio, 2,2,3,3,3-pentafluoropropylthio, heptafluoropropylthio, 1-(fluoromethyl)-2-fluoroethylthio, 1-(chloromethyl)-2-chloroethylthio or 1-(bromomethyl)-2-bromoethylthio. Examples for C₁-C₆-haloalkylthio further encompass 4-fluorobutylthio, 4-chlorobutylthio, 4 bromobutylthio, nonafluorobutylthio, 5-fluoro-1-pentylthio, 5-chloro-1-pentylthio, 5 bromo-1-pentylthio, 5-iodo-1-pentylthio, 5,5,5-trichloro-1-pentylthio, undecafluoropentylthio, 6-fluoro-1-hexylthio, 6-chloro-1-hexylthio, 6-bromo-1-hexylthio, 6-iodo-1-hexylthio, 6,6,6-trichloro-1-hexylthio and dodecafluorohexylthio. Particularly preferred are chloromethyl-thio, fluoromethylthio, difluoromethylthio, trifluoromethylthio, 2-fluoroethylthio, 2-chloroethylthio and 2,2,2-trifluoroethylthio.

The term “C₁-C₆-alkylamino” as used herein and in the term C₁-C₆-alkylaminocarbonyl refers to a secondary amino group carrying one alkyl group as defined above, e.g. methylamino, ethylamino, propylamino, 1-methylethylamino, butylamino, 1-methyl-propylamino, 2-methylpropylamino, 1,1-dimethylethylamino, pentylamino, 1-methylbutylamino, 2-methylbutylamino, 3-methylbutylamino, 2,2-dimethylpropyl-amino, 1-ethylpropylamino, hexylamino, 1,1-dimethylpropylamino, 1,2-dimethylpropyl-amino, 1-methylpentylamino, 2-methylpentylamino, 3-methylpentylamino, 4-methylpentylamino, 1,1-dimethylbutylamino, 1,2-dimethylbutylamino, 1,3-dimethylbutylamino, 2,2-dimethylbutylamino, 2,3-dimethylbutylamino, 3,3-dimethylbutylamino, 1-ethylbutylamino, 2-ethylbutylamino, 1,1,2-trimethylpropylamino, 1,2,2-trimethylpropylamino, 1-ethyl-1-methylpropylamino or 1-ethyl-2-methylpropylamino.

The term “di(C₁-C₆-alkyl)amino)” as used herein and in the term di(C₁-C₆-alkylamino)-carbonyl refers to a tertiary amino group carrying two alkyl radicals as defined above, e.g. dimethylamino, diethylamino, di-n-propylamino, diiso-propylamino, N-ethyl-N-methylamino, N-(n-propyl)-N-methylamino, N-(isopropyl)-N methylamino, N-(n-butyl)-N-methylamino, N-(n-pentyl)-N-methylamino, N-(2-butyl)-N methylamino, N-(isobutyl)-N-methylamino, N-(n-pentyl)-N-methylamino, N-(n-propyl)-N ethylamino, N-(isopropyl)-N-ethylamino, N-(n-butyl)-N-ethylamino, N-(n-pentyl)-N ethylamino, N-(2-butyl)-N-ethylamino, N-(isobutyl)-N-ethylamino and the like.

The term “C₂-C₆-alkenyl” as used herein and in the alkenyl moieties of C₂-C₆-alkenyloxy, C₂-C₆-alkenylamino and C₂-C₆-alkenylcarbonyl refers to a straight-chain or branched unsaturated hydrocarbon group having 2 to 6 carbon atoms and a double bond in any position, such as ethenyl, 1-propenyl, 2-propenyl, 1-methyl-ethenyl, 1-butenyl, 2-butenyl, 3-butenyl, 1-methyl-1-propenyl, 2 methyl-1-propenyl, 1-methyl-2-propenyl, 2-methyl-2-propenyl; 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3 dimethyl-1-butenyl, 3,3-dimethyl-2-butenyl, 1-ethyl-1-butenyl, 1-ethyl-2-butenyl, 1 ethyl-3-butenyl, 2-ethyl-1-butenyl, 2-ethyl-2-butenyl, 2-ethyl-3-butenyl, 1,1,2 trimethyl-2-propenyl, 1-ethyl-1-methyl-2-propenyl, 1-ethyl-2-methyl-1-propenyl and 1-ethyl-2-methyl-2-propenyl.

The term, “C₂-C₆-alkenyloxy” as used herein refers to a straight-chain or branched alkenyl group having 2 to 6 carbon atoms which is attached via an oxygen atom, such as vinyloxy, allyloxy (propen-3-yloxy), methallyloxy, buten-4-yloxy and the like.

The term “C₂-C₆-alkynyl” as used herein and in the alkynyl moieties of C₂-C₆-alkynyloxy, C₂-C₆-alkynylamino, C₂-C₆-alkynylthio and C₂-C₆-alkynyl carbonyl refers to a straight-chain or branched unsaturated hydrocarbon group having 2 to 6 carbon atoms and containing at least one triple bond, such as ethynyl, prop-1-yn-1-yl, prop-2-yn-1-yl, n-but-1-yn-1-yl, n-but-1-yn-3-yl, n-but-1-yn-4-yl, n-but-2-yn-1-yl, n-pent-1-yn-1-yl, n-pent-1-yn-3-yl, n-pent-1-yn-4-yl, n-pent-1-yn-5-yl, n-pent-2-yn-1-yl, n-pent-2-yn-4-yl, n-pent-2-yn-5-yl, 3-methylbut-1-yn-3-yl, 3-methylbut-1-yn-4-yl, n-hex-1-yn-1-yl, n-hex-1-yn-3-yl, n-hex-1 yn-4-yl, n-hex-1-yn-5-yl, n-hex-1-yn-6-yl, n-hex-2-yn-1-yl, n-hex-2-yn-4-yl, n-hex-2-yn-5-yl, n-hex-2-yn-6-yl, n-hex-3-yn-1-yl, n-hex-3-yn-2-yl, 3-methylpent-1-yn-1-yl, 3 methylpent-1-yn-3-yl, 3-methylpent-1-yn-4-yl, 3-methylpent-1-yn-5-yl, 4-methylpent-1-yn-1-yl, 4-methylpent-2-yn-4-yl or 4-methylpent-2-yn-5-yl and the like.

The term, “C₂-C₆-alkynyloxy” as used herein refers to a straight-chain or branched alkynyl group having 2 to 6 carbon atoms (as mentioned above) which is attached via an oxygen atom, such as propargyloxy (propyn-3-yloxy), butyn-3-yloxy, butyn-4-yloxy and the like.

The term “C₃-C₁₂-cycloalkyl” as used herein refers to a mono- or bi- or polycyclic hydrocarbon radical having 3 to 12 (═C₃-C₁₂-cycloalkyl), frequently 3 to 8 carbon atoms (═C₃-C₈-cycloalkyl), in particular 3 to 6 carbon atoms (═C₃-C₆-cycloalkyl). Examples of monocyclic radicals comprise cyclo-propyl, cyclobutyl, cyclopentyl, cyclohexyl, cyclo-heptyl, cyclooctyl, cyclononyl and cyclodecyl. Examples of bicyclic radicals comprise bicyclo[2.2.1]heptyl, bicyclo[3.1.1]heptyl, bicyclo[2.2.2]octyl and bicyclo[3.2.1]octyl.

Phenyl fused to benzene is naphthyl.

Phenyl fused to a 5- or 6-membered non-aromatic (i.e. saturated or partially unsaturated) heterocyclic ring is for example 2,3-dihydrobenzofuranyl, benzoxolanyl, 2,3-dihydrobenzothienyl, indolinyl, chromanyl, chromenyl, benzodioxanyl and the like. Examples for phenyl fused to a 5- or 6-membered aromatic heterocyclic ring (=fused to a 5- or 6-membered heteroaromatic ring) are given below.

The term “heteroaryl” (“mono or bicyclic 5- to 10-membered heteroaromatic ring”) as used herein refers to a monocyclic heteroaromatic radical which has 5 or 6 ring members, which may be fused to a carbocyclic or heterocyclic 5, 6 or 7 membered ring thus having a total number of ring members from 8 to 10, wherein in each case 1, 2, 3 or 4, preferably 1, 2 or 3, of these ring members are heteroatoms selected, independently from each other, from the group consisting of oxygen, nitrogen and sulfur. The heteraryl radical may be attached to the remainder of the molecule via a carbon ring member or via a nitrogen ring member. The carbocyclic or heterocyclic fused ring is selected from C₅-C₇-cycloalkyl, 5 to 7 membered heterocyclyl and phenyl.

Examples for monocyclic 5- to 6-membered heteroaromatic rings include triazinyl, pyrazinyl, pyrimidyl, pyridazinyl, pyridyl, thienyl, furyl, pyrrolyl, pyrazolyl, imidazolyl, triazolyl, tetrazolyl, thiazolyl, oxazolyl, thiadiazolyl, oxadiazolyl, isothiazolyl and isoxazolyl.

Examples for 5- to 6-membered heteroaromatic rings being fused to a phenyl ring (or for benzene fused to a 5- to 6-membered heteroaromatic ring) are quinolinyl, isoquinolinyl, indolyl, indolizinyl, isoindolyl, indazolyl, benzofuryl, benzthienyl, benzo[b]thiazolyl, benzoxazolyl, benzthiazolyl, benzoxazolyl, and benzimidazolyl. Examples for 5- to 6-membered heteroaromatic rings being fused to a cycloalkenyl ring are dihydroindolyl, dihydroindolizinyl, dihydroisoindolyl, dihydrochinolinyl, dihydroisochinolinyl, chromenyl, chromanyl and the like.

The term “heterocycle” (“saturated or partially unsaturated heterocycle”) comprises nonaromatic saturated or partially unsaturated heterocyclic rings having 5 or 6 ring members and 1, 2, 3 or 4, preferably 1, 2 or 3 heteroatoms as ring members. The heterocyclic radical may be attached to the remainder of the molecule via a carbon ring member or via a nitrogen ring member. Examples for non-aromatic rings include pyrrolidinyl, pyrazolinyl, imidazolinyl, pyrrolinyl, pyrazolinyl, imidazolinyl, tetrahydrofuranyl, dihydrofuranyl, 1,3-dioxolanyl, dioxolenyl, thiolanyl, dihydrothienyl, oxazolidinyl, isoxazolidinyl, oxazolinyl, isoxazolinyl, thiazolinyl, isothiazolinyl, thiazolidinyl, isothiazolidinyl, oxathiolanyl, piperidinyl, piperazinyl, pyranyl, dihydropyranyl, tetrahydropyranyl, 1,3- and 1,4-dioxanyl, thiopyranyl, dihydrothiopyranyl, tetrahydrothiopyranyl, morpholinyl, thiazinyl and the like. Examples for heterocyclic ring also comprising 1 or 2 carbonyl groups as ring members comprise pyrrolidin-2-onyl, pyrrolidin-2,5-dionyl, imidazolidin-2-onyl, oxazolidin-2-onyl, thiazolidin-2-onyl and the like.

The term “linear or branched C₁-C₆-alkanediyl” as used herein and in the term C₁-C₆-alkanediyloxy refers to a straight-chain or branched saturated alkyl group having 1 to 6 carbon atoms (as mentioned above) in particular 1 to 4 carbon atoms (═C₁-C₄-alkanediyl), where one of the hydrogen atoms in these groups is replaced by a further bonding position. Examples for linear C₁-C₆-alkanediyl comprise methyldiyl, ethane-1,2-diyl, propane-1,3-diyl, butane-1,4-diyl, pentane-1,5-diyl, hexane-1,6-diyl. Examples for branched C₁-C₆-alkanediyl comprise ethyl-1,1-diyl, propyl-1,1-diyl, butyl-1,1-diyl, 1-methylethane-1,2 diyl, 1,2-dimethylethane-1,2-diyl, 1-ethylethane-1,2 diyl, 1-methylpropane-1,3-diyl, 2-methylpropan-1,3-diyl and the like.

### Preferences

The remarks made below concerning preferred embodiments of the variables of the compounds of formulae I and II, of the features of the use and method according to the invention and of the composition of the invention are valid on their own as well as preferably in combination with each other.

The group Ar preferably is phenyl, which is unsubstituted or carries 1, 2 or 3 radicals R^{Ar}, wherein R^{Ar} is as defined above and wherein
R^{Ar} preferably is selected from halogen, CN, C₁-C₆-alkyl, C₃-C₈-cycloalkyl, formyl, C₁-C₆-alkylcarbonyl, —C(═O)OH, C₁-C₆-alkoxycarbonyl, —C(═O)NH₂, C₁-C₆-alkylaminocarbonyl, di(C₁-C₆-alkyl)aminocarbonyl, —OH, C₁-C₆-alkoxy, —SH, C₁-C₆-alkylthio, —NH₂, C₁-C₆-alkylamino and di(C₁-C₆-alkyl)amino, wherein each carbon atom of the aforementioned radicals may carry any combination of 1, 2 or 3 substituents, independently of one another selected from halogen, CN, NO₂, OH, C₁-C₆-alkoxy, C₁-C₆-haloalkoxy, C₁-C₆-alkylthio, and/or two radicals R^{Ar} bound to adjacent carbon atoms of the phenyl ring together with said carbon atoms form a fused benzene, a fused saturated or partially unsaturated 5 or 6-membered carbocycle or a fused 5- or 6-membered heterocycle, which contains 1, 2, 3 or 4 heteroatoms selected from O, S and N as ring members, wherein each fused benzene, carbocycle or heterocycle is unsubstituted or carries 1, 2 3 or 4 substituents, independently of one another selected from halogen, CN, NO₂, OH, SH, NH₂, COOH, C₁-C₆-alkyl, C₁-C₆-alkoxy, C₂-C₆-alkenyloxy, C₂-C₆-alkynyloxy, C₁-C₆-haloalkoxy and C₁-C₆-alkylthio.

More preferably the radical R^{Ar} is selected from halogen, CN, C₁-C₆-alkyl, C₁-C₆-haloalkyl, C₁-C₆-alkoxy and C₁-C₆-haloalkoxy.

Likewise preferred are azolin-2-ylamino compounds of formula I, wherein Ar is phenyl, which carries 2 radicals R^{Ar} in the 2- and 3-position, relative to the bonding position, and wherein R^{Ar} has one of the meanings given before, in particular one of the preferred or more preferred meanings.

More preferred are azolin-2-ylamino compounds of formula I, wherein Ar is phenyl, which carries 2 radicals R^{Ar} selected from halogen, CN, C₁-C₆-alkyl, C₁-C₆-haloalkyl, C₁-C₆-alkoxy and C₁-C₆-haloalkoxy in the 2- and 3-position, relative to the bonding position of phenyl.

More preferred are azolin-2-ylamino compounds of formula I, wherein Ar is phenyl which carries 2 radicals R^{Ar} selected from halogen, especially fluorine, chlorine, or bromine, C₁-C₄-alkyl, C₁-C₄-alkoxy, C₁-C₄-haloalkyl, or 2 groups R² which are bound to adjacent carbon atoms of the phenyl ring and together form a group —O—CH₂CH₂—, —O—CH₂CH₂CH₂—, —O—CH₂—O—, —O—CH₂CH₂—O—, —(CH₂)₃—, —(CH₂)₄— or —CH═CH—CH═CH—.

Special preference is given to compounds of formula I, wherein Ar is phenyl which carries 2 radicals R^{Ar} selected from chlorine and C₁-C₄-alkyl, especially methyl, or 2 radicals R^{Ar} which are bound to adjacent carbon atoms of the phenyl ring and together form a group —O—CH₂CH₂—, —O—CH₂CH₂CH₂—, —O—CH₂—O—, —O—CH₂CH₂—O—, —(CH₂)₃—, —(CH₂)₄—, or —CH═CH—CH═CH—, wherein the 2 radicals R^{Ar} preferably are in the 2- and 3-position, relative to the bonding position of phenyl.

Preferred are azolin-2-ylamino compounds of formula I wherein A is a radical of formula A2.

The radical R¹ preferably is selected from hydrogen, halogen, C₁-C₆-alkyl, C₂-C₆-alkenyl, C₂-C₆-alkynyl and C₃-C₆-cycloalkyl, wherein each carbon atom of the aforementioned radicals is unsubstituted or carries any combination of 1, 2 or 3 substituents selected independently of one another from halogen, CN, NO₂, —OH, C₁-C₆-alkoxy, C₁-C₆-haloalkoxy, C₂-C₆-alkenyloxy, C₂-C₆-alkynyloxy, NH₂, C₁-C₆-alkylamino, di(C₁-C₆-alkyl)amino, —SH and C₁-C₆-alkylthio, and wherein C₃-C₆-cycloalkyl additionally may carry 1, 2 or 3 substituents selected independently of one another from C₁-C₆-alkyl, C₂-C₆-alkenyl, C₂-C₆-alkynyl and C₁-C₆-haloalkyl.

More preferably the radical R¹ is selected from hydrogen and C₁-C₆-alkyl. Most preferably R¹ is hydrogen.

The radicals R² and R³ preferably are independently of one another selected from hydrogen, halogen, C₁-C₆-alkyl, C₂-C₆-alkenyl, C₂-C₆-alkynyl and C₃-C₆-cycloalkyl, wherein each carbon atom of the aforementioned radicals is unsubstituted or carries any combination of 1, 2 or 3 substituents selected independently of one another from halogen, CN, NO₂, —OH, C₁-C₆-alkoxy, C₁-C₆-haloalkoxy, C₂-C₆-alkenyloxy, C₂-C₆-alkynyloxy, NH₂, C₁-C₆-alkylamino, di(C₁-C₆-alkyl)amino, —SH and C₁-C₆-alkylthio, and wherein C₃-C₆-cycloalkyl additionally may carry 1, 2 or 3 substituents selected independently of one another from C₁-C₆-alkyl, C₂-C₆-alkenyl, C₂-C₆-alkynyl and C₁-C₆-haloalkyl.

More preferably R² and R³ are independently of one another selected from hydrogen, halogen and C₁-C₆-alkyl. Most preferably the radicals R² and R³ are hydrogen.

The number n of —C(R²)(R³)— groups preferably is 1 or 2. More preferably n is 1.

According to one preferred embodiment of the invention the group —[C(R²)(R³)]ₙ— is methylene, i.e. the radicals R² and R³ both are hydrogen and n is 1.

The radical R⁴ preferably is selected from hydrogen, halogen, C₁-C₆-alkyl, C₂-C₆-alkenyl, C₂-C₆-alkynyl, C₃-C₈-cycloalkyl, Ar⁵, —C(═O)R^{a}, —C(═S)R^{a}, —C(═O)OR^{b} and —C(═O)NR^{c}R^{d}, wherein C₁-C₆-alkyl, C₁-C₆-alkoxy, C₂-C₆-alkenyl, C₂-C₆-alkenyloxy, C₂-C₆-alkynyl, C₂-C₆-alkynyloxy and C₃-C₈-cycloalkyl are unsubstituted or carry any combination of 1, 2, 3, 4 or 5 substituents, independently of one another selected from halogen, CN, NO₂, —OH, C₁-C₆-alkoxy, C₁-C₆-haloalkoxy, C₂-C₆-alkenyloxy, C₂-C₆-alkynyloxy, NH₂, C₁-C₆-alkylamino, di(C₁-C₆-alkyl)amino, —SH, C₁-C₆-alkylthio, Ar⁶, —CH₂—Ar⁶, —O—Ar⁶ and —S—Ar⁶, wherein Ar⁵ and Ar⁶ have one of the meanings given before, and wherein C₃-C₈-cycloalkyl additionally may carry 1, 2 or 3 substituents selected independently of one another from C₁-C₆-alkyl, C₂-C₆-alkenyl, C₂-C₆-alkynyl and C₁-C₆-haloalkyl.

More preferably the radical R⁴ is selected from hydrogen, C₁-C₆-alkyl, Ar⁵, —C(═O)R^{a}, —C(═S)R^{a}, —C(═O)OR^{b} and —C(═O)NR^{c}R^{d}, wherein C₁-C₆-alkyl may carry any combination of 1, 2, 3, 4 or 5 substituents, independently of one another selected from halogen, C₁-C₆-alkoxy, C₁-C₆-haloalkoxy and Ar⁶, wherein Ar⁵ and Ar⁶ have one of the meanings given before.

Most preferably the radical R⁴ is selected from C₁-C₆-alkyl and Ar⁵, wherein C₁-C₆-alkyl may carry any combination of 1, 2, 3, 4 or 5 substituents, independently of one another selected from halogen, C₁-C₆-alkoxy, C₁-C₆-haloalkoxy and Ar⁶, wherein Ar⁵ and Ar⁶ have one of the meanings given before.

The radicals Ar⁵ and Ar⁶ if present in the radicals R⁴, as defined above, are preferably independently of one another selected from phenyl, wherein phenyl is unsubstituted or carries any combination of 1, 2, 3, 4 or 5 substituents, independently of one another selected from halogen, C₁-C₆-alkyl, C₁-C₆-haloalkyl, C₁-C₆-alkoxy and C₁-C₆-haloalkoxy.

One particular embodiment of the invention relates to compounds of formulae I or II, wherein X is sulfur.

Another particular embodiment of the invention relates to compounds of formulae I or II, wherein X is oxygen.

Another particular embodiment of the invention relates to compounds of formulae I or II wherein X is N(R⁸).

Preferably X is O or S. More preferably X is S.

The radicals R^{7a}, R^{7b}, R^{7c}, R^{7d} preferably are independently of one another selected from hydrogen, halogen, C₁-C₆-alkyl and C₁-C₆-haloalkyl. More preferably the radicals R^{7a}, R^{7b}, R^{7c} and R^{7d} are hydrogen.

The radicals R⁵, R⁶, R⁸ preferably are selected independently of one another from hydrogen, CN, NO₂, C₁-C₆-alkyl, C₂-C₆-alkenyl, C₂-C₆-alkynyl and C₃-C₈-cycloalkyl, wherein C₁-C₆-alkyl, C₂-C₆-alkenyl, C₂-C₆-alkynyl and C₃-C₈-cycloalkyl are unsubstituted or carry any combination of 1, 2 or 3 or substituents, independently of one another selected from halogen, CN, NO₂, —OH, C₁-C₆-alkoxy, C₂-C₆-alkenyloxy, C₂-C₆-alkynyloxy, C₁-C₆-haloalkoxy, —SH, C₁-C₆-alkylthio, C₁-C₆-haloalkylthio, —C(═O)H, C₁-C₆-alkylcarbonyl, C₁-C₆-haloalkylcarbonyl, saturated or partially unsaturated 5, 6 or 7-membered heterocycles containing 1, 2, 3 or 4 heteroatoms selected independently from one another from O, S, N as ring members, —Ar⁴ or —CH₂—Ar⁴, wherein Ar⁴ has one of the meanings given before, and wherein C₃-C₈-cycloalkyl additionally may carry any combination of 1, 2 or 3 or substituents, independently of one another selected from C₁-C₆-alkyl, C₁-C₆-haloalkyl, C₂-C₆-alkenyl and C₂-C₆-alkynyl, and likewise preferred R⁵, R⁶, R⁸ may further be selected from -E-C(═O)R^{a}, -E-C(═O)OR^{b}, -E-C(═O)SR^{e}, -E-C(═S)OR^{b}, -E-C(═S)NR^{c}R^{d}, -E-C(═S)SR^{e}, -E-OR^{b}, -E-SR^{e}, —S(═O)R^{e}, E-NR^{c}R^{d}, —S(═O)₂R^{e} and —S(═O)₂OR^{b}, wherein

E is a single bond or —CH₂—, and wherein R^{a}, R^{b}, R^{c}, R^{d} and R^{e} independently of one another have one of the meanings given before.

More preferably the radicals R⁵, R⁶ and R⁸ are independently of one another selected from hydrogen, CN, C₁-C₆-alkyl, —C(═O)R^{a}, —C(═O)OR^{b}, —OR^{b} and -E-NR^{c}R^{d}, wherein R^{a}, R^{b} R^{c} and R^{d} are selected independently of one another from hydrogen, C₁-C₆-alkyl and phenyl, wherein C₁-C₆-alkyl is unsubstituted or may carry any combination of 1, 2, 3, 4 or 5 substituents, independently of one another selected from halogen, CN, NO₂, OH, C₁-C₆-alkoxy, C₁-C₆-haloalkoxy and C₁-C₆-alkylthio and wherein phenyl is unsubstituted 1, 2, 3, 4 or 5 substituents, independently of one another selected from halogen, C₁-C₆-alkyl, C₁-C₆-haloalkyl, C₁-C₆-alkoxy and C₁-C₆-haloalkoxy. Most preferably the radicals R⁵, R⁶ and R⁸ are hydrogen.

The radicals R^{a}, R^{a′}, R^{b}, R^{c}, R^{d}, R^{e}, R^{f}, R^{g} and R^{h} preferably are selected each independently of one another from hydrogen, C₁-C₆-alkyl and phenyl, wherein C₁-C₆-alkyl is unsubstituted or may carry any combination of 1, 2, 3, 4 or 5 substituents, independently of one another selected from halogen, CN, NO₂, OH, C₁-C₆-alkoxy, C₁-C₆-haloalkoxy and C₁-C₆-alkylthio and wherein phenyl is unsubstituted 1, 2, 3, 4 or 5 substituents, independently of one another selected from halogen, C₁-C₆-alkyl, C₁-C₆-haloalkyl, C₁-C₆-alkoxy and C₁-C₆-haloalkoxy.

The radicals Ar¹, Ar², Ar³, Ar⁴, Ar⁵, Ar⁶, Ar⁷, Ar⁸, Ar⁹, and Ar¹⁰ are preferably independently of one another selected from phenyl, wherein phenyl is unsubstituted or carries any combination of 1, 2, 3, 4 or 5 substituents, independently of one another selected from halogen, C₁-C₆-alkyl, C₁-C₆-haloalkyl, C₁-C₆-alkoxy and C₁-C₆-haloalkoxy.

The compounds of formula II can be converted into compounds of formula I by means of a chemical reaction. Thus, the definitions given hereinbefore for Ar, R^{Ar}, R¹, R², R³, R⁴, R⁵, R⁶, X, R^{7a}, R^{7b}, R^{7c}, R^{7d}, R⁸, B, R^{a}, R^{a′}, R^{b}, R^{c}, R^{d}, R^{e}, R^{f}, R^{g}, R^{h}, Ar¹, A², AR³, Ar⁴, Ar⁵, Ar⁶, Ar⁷, Ar⁸, Ar⁹, and Ar¹⁰ apply for compounds of formula I as well as for the compounds of formula II. In both cases the remarks made hereinbefore concerning preferred meanings of those variables are valid on their own as well as preferably in combination with each other. For the compounds of formula II the remarks made hereinbefore concerning preferred meanings of those variables are further preferably valid in combination with the preferred meanings given for the radicals R⁹, Rⁱ, Ar¹¹ or Ar¹².

One specific embodiment relates to compounds of formula (II) wherein W is a group —OR⁹, and R⁹ is hydrogen or —C(═O)—R.

The radical Rⁱ, if present in the radical R⁹, preferably is selected from hydrogen, C₁-C₆-alkyl, and Ar¹¹, wherein C₁-C₆-alkyl is unsubstituted or carries any combination of 1, 2 or 3 substituents, independently of one another selected from halogen, C₁-C₆-alkoxy, C₁-C₆-haloalkoxy and Ar¹², wherein Ar¹¹ and Ar¹² have one of the meanings given before.

The radicals Ar¹¹ and Ar¹², if present in the radical Rⁱ, are independently of one another selected from phenyl, wherein phenyl is unsubstituted or carries any combination of 1, 2, 3, 4 or 5 substituents, independently of one another selected from halogen, C₁-C₆-alkyl, C₁-C₆-haloalkyl and C₁-C₆-haloalkoxy.

More preferably the radical R⁹ preferably is selected from hydrogen and —C(═O)—Rⁱ, wherein
Rⁱ is selected from hydrogen, C₁-C₆-alkyl, and Ar¹¹, wherein C₁-C₆-alkyl is unsubstituted or carries any combination of 1, 2 or 3 substituents, independently of one another selected from halogen, C₁-C₆-alkoxy, C₁-C₆-haloalkoxy and Ar¹², wherein
   Ar¹¹ and Ar¹² are independently of one another selected from phenyl, wherein phenyl is unsubstituted or carries any combination of 1, 2, 3, 4 or 5 substituents, independently of one another selected from halogen, C₁-C₆-alkyl, C₁-C₆-haloalkyl and C₁-C₆-haloalkoxy.

Most preferably the radical R⁹ is selected from hydrogen and C₁-C₆-alkylcarbonyl.

One specific embodiment of the invention relates to azolin-2-ylamino compounds of formula I, wherein

n is 1 or 2, preferably 1;

Ar is phenyl, which carries 1, 2 or 3 radicals R^{Ar}, wherein
R^{Ar} is selected from halogen, CN, C₁-C₆-alkyl, C₃-C₈-cycloalkyl, formyl, C₁-C₆-alkylcarbonyl, —C(═O)OH, C₁-C₆-alkoxycarbonyl, —C(═O)NH₂, C₁-C₆-alkylaminocarbonyl, di(C₁-C₆-alkyl)aminocarbonyl, —OH, C₁-C₆-alkoxy, —SH, C₁-C₆-alkylthio, —NH₂, C₁-C₆-alkylamino and di(C₁-C₆-alkyl)amino, wherein each carbon atom of the aforementioned radicals may carry any combination of 1, 2 or 3 substituents, independently of one another selected from halogen, CN, NO₂, OH, C₁-C₆-alkoxy, C₁-C₆-haloalkoxy, C₁-C₆-alkylthio, and/or two radicals R^{Ar} bound to adjacent carbon atoms of the phenyl or naphtyl ring together with said carbon atoms form a fused benzene, a fused saturated or partially unsaturated 5 or 6-membered carbocycle or a fused 5- or 6-membered heterocycle, which contains 1, 2, 3 or 4 heteroatoms selected from O, S and N as ring members, wherein each fused benzene, carbocycle or heterocycle is unsubstituted or carries 1, 2, 3 or 4 substituents, independently of one another selected from halogen, CN, NO₂, OH, SH, NH₂, COOH, C₁-C₆-alkyl, C₁-C₆-alkoxy, C₂-C₆-alkenyloxy, C₂-C₆-alkynyloxy, C₁-C₆-haloalkoxy and C₁-C₆-alkylthio; and wherein
   R^{Ar} is preferably selected from halogen, CN, C₁-C₆-alkyl, C₁-C₆-haloalkyl, C₁-C₆-alkoxy and C₁-C₆-haloalkoxy; and wherein R^{Ar} is likewise preferably located in the 2- and 3-position, relative to the bonding position, relative to the bonding position of phenyl;

A is a radical of the formulae A.1 or A.2

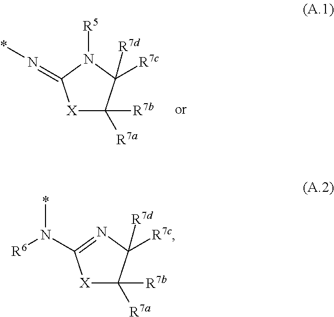

wherein
* denotes the point of attachments to the remaining part of the molecule;
X is S, O or N(R⁸), preferably S or O;
R^{7a}, R^{7b}, R^{7c} and R^{7d} are independently of one another selected from hydrogen, halogen, C₁-C₆-alkyl and C₁-C₆-haloalkyl, in particular hydrogen;
R⁵, R⁶ and R⁸ are independently of one another selected from hydrogen, CN, C₁-C₆-alkyl, —C(═O)R^{a}, —C(═O)OR^{b} and —OR^{b}, wherein R^{a} and R^{b} are selected independently of one another from hydrogen, C₁-C₆-alkyl and phenyl, wherein C₁-C₆-alkyl is unsubstituted or may carry any combination of 1, 2, 3, 4 or 5 substituents, independently of one another selected from halogen, CN, NO₂, OH, C₁-C₆-alkoxy, C₁-C₆-haloalkoxy and C₁-C₆-alkylthio and wherein phenyl is unsubstituted 1, 2, 3, 4 or 5 substituents, independently of one another selected from halogen, C₁-C₆-alkyl, C₁-C₆-haloalkyl, C₁-C₆-alkoxy and C₁-C₆-haloalkoxy; in particular R⁵, R⁶ and are hydrogen;
R¹ is selected from hydrogen, C₁-C₆-alkyl, C₂-C₆-alkenyl, C₂-C₆-alkynyl and C₃-C₆-cycloalkyl, wherein each carbon atom of the aforementioned radicals is un-substituted or carries any combination of 1, 2 or 3 substituents selected independently of one another from halogen, CN, NO₂, —OH, C₁-C₆-alkoxy, C₁-C₆-haloalkoxy, C₂-C₆-alkenyloxy, C₂-C₆-alkynyloxy, NH₂, C₁-C₆-alkylamino, di(C₁-C₆-alkyl)amino, —SH and C₁-C₆-alkylthio, and wherein C₃-C₆-cycloalkyl additionally may carry 1, 2 or 3 substituents selected independently of one another from C₁-C₆-alkyl, C₂-C₆-alkenyl, C₂-C₆-alkynyl and C₁-C₆-haloalkyl; and wherein
   R¹ is preferably selected from hydrogen and C₁-C₆-alkyl and more preferably is hydrogen;
R² and R³ are selected independently of one another from hydrogen, C₁-C₆-alkyl, C₂-C₆-alkenyl, C₂-C₆-alkynyl and C₃-C₆-cycloalkyl, wherein each carbon atom of the aforementioned radicals is unsubstituted or carries any combination of 1, 2 or 3 substituents selected independently of one another from halogen, CN, NO₂, —OH, C₁-C₆-alkoxy, C₁-C₆-haloalkoxy, C₂-C₆-alkenyloxy, C₂-C₆-alkynyloxy, NH₂, C₁-C₆-alkylamino, di(C₁-C₆-alkyl)amino, —SH and C₁-C₆-alkylthio, and wherein C₃-C₆-cycloalkyl additionally may carry 1, 2 or 3 substituents selected independently of one another from C₁-C₆-alkyl, C₂-C₆-alkenyl, C₂-C₆-alkynyl and C₁-C₆-haloalkyl; and wherein
   R² and R³ are preferably selected from hydrogen and C₁-C₆-alkyl and more preferably are hydrogen;
R⁴ is selected from hydrogen, halogen, C₁-C₆-alkyl and Ar⁵, wherein C₁-C₆-alkyl is unsubstituted or carries any combination of 1, 2, 3, 4 or 5 substituents, independently of one another selected from halogen, C₁-C₆-alkoxy, C₁-C₆-haloalkoxy and Ar⁶, wherein
   Ar⁵ and Ar⁶ are independently of one another selected from phenyl, wherein phenyl is unsubstituted or carries any combination of 1, 2, 3, 4 or 5 substituents, independently of one another selected from halogen, C₁-C₆-alkyl, C₁-C₆-haloalkyl, C₁-C₆-alkoxy and C₁-C₆-haloalkoxy;
      or the enantiomers, diastereomers or salts thereof.

Another specific embodiment of the invention relates to compounds of formula II, wherein

n is 1 or 2, preferably 1;

Ar is phenyl, which carries 1, 2 or 3 radicals R^{a}, wherein
R^{Ar} is selected from halogen, CN, C₁-C₆-alkyl, C₃-C₈-cycloalkyl, formyl, C₁-C₆-alkylcarbonyl, —C(═O)OH, C₁-C₆-alkoxycarbonyl, —C(═O)NH₂, C₁-C₆-alkylaminocarbonyl, di(C₁-C₆-alkyl)aminocarbonyl, —OH, C₁-C₆-alkoxy, —SH, C₁-C₆-alkylthio, —NH₂, C₁-C₆-alkylamino and di(C₁-C₆-alkyl)amino, wherein each carbon atom of the aforementioned radicals may carry any combination of 1, 2 or 3 substituents, independently of one another selected from halogen, CN, NO₂, OH, C₁-C₆-alkoxy, C₁-C₆-haloalkoxy, C₁-C₆-alkylthio, and/or
   two radicals R^{Ar} bound to adjacent carbon atoms of the phenyl or naphtyl ring together with said carbon atoms form a fused benzene, a fused saturated or partially unsaturated 5 or 6-membered carbocycle or a fused 5- or 6-membered heterocycle, which contains 1, 2, 3 or 4 heteroatoms selected from O, S and N as ring members, wherein each fused benzene, carbocycle or heterocycle is unsubstituted or carries 1, 2, 3 or 4 substituents, independently of one another selected from halogen, CN, NO₂, OH, SH, NH₂, COOH, C₁-C₆-alkyl, C₁-C₆-alkoxy, C₂-C₆-alkenyloxy, C₂-C₆-alkynyloxy, C₁-C₆-haloalkoxy and C₁-C₆-alkylthio; and wherein
   R^{Ar} is preferably selected from halogen, CN, C₁-C₆-alkyl, C₁-C₆-haloalkyl, C₁-C₆-alkoxy and C₁-C₆-haloalkoxy; and wherein R^{Ar} is likewise preferably located in the 2- and 3-position, relative to the bonding position, relative to the bonding position of phenyl;

B is a radical

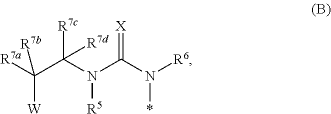

wherein
* denotes the point of attachment to the remaining part of the molecule;
X is S, O or N(R⁸), preferably S or O;
R^{7a}, R^{7b}, R^{7c} and R^{7d} are independently of one another selected from hydrogen, halogen, C₁-C₆-alkyl and C₁-C₆-haloalkyl, in particular hydrogen;
R⁵, R⁶ and R⁸ are independently of one another selected from hydrogen, CN, C₁-C₆-alkyl, —C(═O)R^{a}, —C(═O)OR^{b} and —OR^{b}, wherein R^{a} and R^{b} are selected independently of one another from hydrogen, C₁-C₆-alkyl and phenyl, wherein C₁-C₆-alkyl is unsubstituted or may carry any combination of 1, 2, 3, 4 or 5 substituents, independently of one another selected from halogen, CN, NO₂, OH, C₁-C₆-alkoxy, C₁-C₆-haloalkoxy and C₁-C₆-alkylthio and wherein phenyl is unsubstituted 1, 2, 3, 4 or 5 substituents, independently of one another selected from halogen, C₁-C₆-alkyl, C₁-C₆-haloalkyl, C₁-C₆-alkoxy and C₁-C₆-haloalkoxy; in particular R⁵, R⁶ and Ware hydrogen;
R¹ is selected from hydrogen, C₁-C₆-alkyl, C₂-C₆-alkenyl, C₂-C₆-alkynyl and C₃-C₆-cycloalkyl, wherein each carbon atom of the aforementioned radicals is un-substituted or carries any combination of 1, 2 or 3 substituents selected independently of one another from halogen, CN, NO₂, —OH, C₁-C₆-alkoxy, C₁-C₆-haloalkoxy, C₂-C₆-alkenyloxy, C₂-C₆-alkynyloxy, NH₂, C₁-C₆-alkylamino, di(C₁-C₆-alkyl)amino, —SH and C₁-C₆-alkylthio, and wherein C₃-C₆-cycloalkyl additionally may carry 1, 2 or 3 substituents selected independently of one another from C₁-C₆-alkyl, C₂-C₆-alkenyl, C₂-C₆-alkynyl and C₁-C₆-haloalkyl; and wherein
   R¹ is preferably selected from hydrogen and C₁-C₆-alkyl, and more preferably is hydrogen;
R² and R³ are selected independently of one another from hydrogen, C₁-C₆-alkyl, C₂-C₆-alkenyl, C₂-C₆-alkynyl and C₃-C₆-cycloalkyl, wherein each carbon atom of the aforementioned radicals is unsubstituted or carries any combination of 1, 2 or 3 substituents selected independently of one another from halogen, CN, NO₂, —OH, C₁-C₆-alkoxy, C₁-C₆-haloalkoxy, C₂-C₆-alkenyloxy, C₂-C₆-alkynyloxy, NH₂, C₁-C₆-alkylamino, di(C₁-C₆-alkyl)amino, —SH and C₁-C₆-alkylthio, and wherein C₃-C₆-cycloalkyl additionally may carry 1, 2 or 3 substituents selected independently of one another from C₁-C₆-alkyl, C₂-C₆-alkenyl, C₂-C₆-alkynyl and C₁-C₆-haloalkyl; and wherein
   R² and R³ are preferably selected from hydrogen and C₁-C₆-alkyl, and more preferably are hydrogen;
R⁴ is selected from hydrogen, halogen, C₁-C₆-alkyl and Ar⁵, wherein C₁-C₆-alkyl is unsubstituted or carries any combination of 1, 2, 3, 4 or 5 substituents, independently of one another selected from halogen, C₁-C₆-alkoxy, C₁-C₆-haloalkoxy and Ar⁶, wherein
   Ar⁵ and Ar⁶ are independently of one another selected from phenyl, wherein phenyl is unsubstituted or carries any combination of 1, 2, 3, 4 or 5 substituents, independently of one another selected from halogen, C₁-C₆-alkyl, C₁-C₆-haloalkyl, C₁-C₆-alkoxy and C₁-C₆-haloalkoxy;

W is selected from halogen and —OR⁹ and preferably is —OR⁹, wherein
R⁹ is selected from hydrogen and —C(═O)—Rⁱ, and wherein
Rⁱ is selected from hydrogen, C₁-C₆-alkyl, and Ar¹¹, wherein C₁-C₆-alkyl is un-substituted or carries any combination of 1, 2 or 3 substituents, independently of one another selected from halogen, C₁-C₆-alkoxy, C₁-C₆-haloalkoxy and Ar¹², wherein
   Ar¹¹ and Ar¹² are independently of one another selected from phenyl and pyridyl, wherein phenyl and pyridyl are each unsubstituted or carry any combination of 1, 2, 3, 4 or 5 substituents, independently of one another selected from halogen, C₁-C₆-alkyl, C₁-C₆-haloalkyl and C₁-C₆-haloalkoxy;
   wherein
   R⁹ most preferably is selected from hydrogen and C₁-C₆-alkylcarbonyl;
      or the enantiomers, diastereomers or salts thereof.

Examples of particularly preferred compounds of the general formulae I and II are the compounds compiled in Tables A to H below. Moreover, the meanings mentioned for the individual variables in the tables are per se, independently of the combination in which they are mentioned, a particularly preferred embodiment of the substituents in question.

### Table A:

Examples of preferred compounds of formula I are the compounds of formula I.A.1, I.A.2, I.A.3 and I.A.4

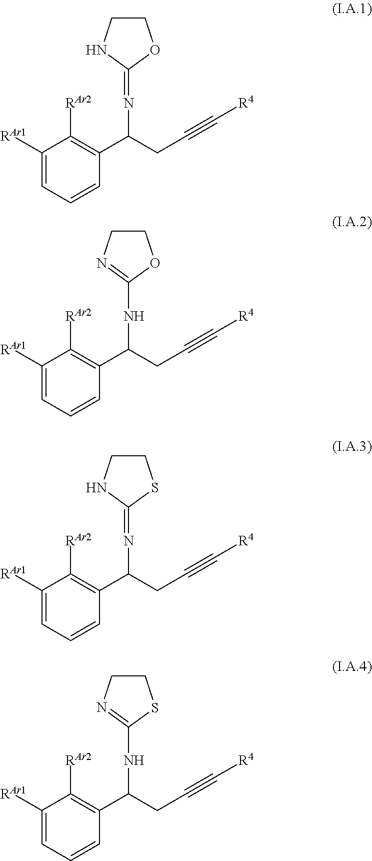

wherein R⁴, R^{Ar1} and R^{Ar2} have the meanings corresponding to one line of the following table 1 (compounds I.A.1.1 to I.A.1.1300, I.A.2.1 to I.A.2.1300, I.A.3.1 to I.A.3.1300 and I.A.4.1 to I.A.4.1300).

TABLE 1

| line | R⁴ | —R^{Ar1}—R^{Ar2}— | |
|---|---|---|---|
| 1 | F | —(CH₂)₃— | |
| 2 | Cl | —(CH₂)₃— | |
| 3 | CH₃ | —(CH₂)₃— | |
| 4 | C₂H₅ | —(CH₂)₃— | |
| 5 | (CH₂)₂CH₃ | —(CH₂)₃— | |
| 6 | CH(CH₃)₂ | —(CH₂)₃— | |
| 7 | (CH₂)₃CH₃ | —(CH₂)₃— | |
| 8 | CH₂CH(CH₃)₂ | —(CH₂)₃— | |
| 9 | C(CH₃)₃ | —(CH₂)₃— | |
| 10 | C₆H₅ | —(CH₂)₃— | |
| 11 | 2-F—C₆H₄ | —(CH₂)₃— | |
| 12 | 2-Cl—C₆H₄ | —(CH₂)₃— | |
| 13 | 2-CH₃—C₆H₄ | —(CH₂)₃— | |
| 14 | 2-CF₃—C₆H₄ | —(CH₂)₃— | |
| 15 | 2-OCH₃—C₆H₄ | —(CH₂)₃— | |
| 16 | 3-F—C₆H₄ | —(CH₂)₃— | |
| 17 | 3-Cl—C₆H₄ | —(CH₂)₃— | |
| 18 | 3-CH₃—C₆H₄ | —(CH₂)₃— | |
| 19 | 3-CF₃—C₆H₄ | —(CH₂)₃— | |
| 20 | 3-OCH₃—C₆H₄ | —(CH₂)₃— | |
| 21 | 4-F—C₆H₄ | —(CH₂)₃— | |
| 22 | 4-Cl—C₆H₄ | —(CH₂)₃— | |
| 23 | 4-CH₃—C₆H₄ | —(CH₂)₃— | |
| 24 | 4-CF₃—C₆H₄ | —(CH₂)₃— | |
| 25 | 4-OCH₃—C₆H₄ | —(CH₂)₃— | |
| 26 | 3,5-F₂—C₆H₃ | —(CH₂)₃— | |
| 27 | 3-F-5-Cl—C₆H₃ | —(CH₂)₃— | |
| 28 | 3-F-5-CH₃—C₆H₃ | —(CH₂)₃— | |
| 29 | 3-F-5-CF₃—C₆H₃ | —(CH₂)₃— | |
| 30 | 3-F-5-OCH₃—C₆H₃ | —(CH₂)₃— | |
| 31 | 3,5-Cl₂—C₆H₃ | —(CH₂)₃— | |
| 32 | 3-Cl-5-CH₃—C₆H₃ | —(CH₂)₃— | |
| 33 | 3-Cl-5-CF₃—C₆H₃ | —(CH₂)₃— | |
| 34 | 3-Cl-5-OCH₃—C₆H₃ | —(CH₂)₃— | |
| 35 | 3,5-(CH₃)₂—C₆H₃ | —(CH₂)₃— | |
| 36 | 3-CH₃-5-CF₃—C₆H₃ | —(CH₂)₃— | |
| 37 | 3-CH₃-5-OCH₃—C₆H₃ | —(CH₂)₃— | |
| 38 | 3,5-(CF₃)₂—C₆H₃ | —(CH₂)₃— | |
| 39 | 3-CF₃-5-OCH₃—C₆H₃ | —(CH₂)₃— | |
| 40 | 3,5-(OCH₃)₂—C₆H₃ | —(CH₂)₃— | |
| 41 | 2,4-F₂—C₆H₃ | —(CH₂)₃— | |
| 42 | 2-F-4-Cl—C₆H₃ | —(CH₂)₃— | |
| 43 | 2-F-4-CH₃—C₆H₃ | —(CH₂)₃— | |
| 44 | 2-F-4-CF₃—C₆H₃ | —(CH₂)₃— | |
| 45 | 2-F-4-OCH₃—C₆H₃ | —(CH₂)₃— | |
| 46 | 2-Cl-4-F—C₆H₃ | —(CH₂)₃— | |
| 47 | 2,4-Cl₂—C₆H₃ | —(CH₂)₃— | |
| 48 | 2-Cl-4-CH₃—C₆H₃ | —(CH₂)₃— | |
| 49 | 2-Cl-4-CF₃—C₆H₃ | —(CH₂)₃— | |
| 50 | 2-Cl-4-OCH₃—C₆H₃ | —(CH₂)₃— | |
| 51 | 2-CH₃-4-F—C₆H₃ | —(CH₂)₃— | |
| 52 | 2-CH₃-4-Cl—C₆H₃ | —(CH₂)₃— | |
| 53 | 2,4-(CH₃)₂—C₆H₃ | —(CH₂)₃— | |
| 54 | 2-CH₃-4-CF₃—C₆H₃ | —(CH₂)₃— | |
| 55 | 2-CH₃-4-OCH₃—C₆H₃ | —(CH₂)₃— | |
| 56 | 2-CF₃-4-F—C₆H₃ | —(CH₂)₃— | |
| 57 | 2-CF₃-4-Cl—C₆H₃ | —(CH₂)₃— | |
| 58 | 2-CF₃-4-CH₃—C₆H₃ | —(CH₂)₃— | |
| 59 | 2,4-(CF₃)₂—C₆H₃ | —(CH₂)₃— | |
| 60 | 2-CF₃-4-OCH₃—C₆H₃ | —(CH₂)₃— | |
| 61 | 2-OCH₃-4-F—C₆H₃ | —(CH₂)₃— | |
| 62 | 2-OCH₃-4-Cl—C₆H₃ | —(CH₂)₃— | |
| 63 | 2-OCH₃-4-CH₃—C₆H₃ | —(CH₂)₃— | |
| 64 | 2-OCH₃-4-CF₃—C₆H₃ | —(CH₂)₃— | |
| 65 | 2,4-(OCH₃)₂—C₆H₃ | —(CH₂)₃— | |
| 66 | F | —(CH₂)₄— | |
| 67 | Cl | —(CH₂)₄— | |
| 68 | CH₃ | —(CH₂)₄— | |
| 69 | C₂H₅ | —(CH₂)₄— | |
| 70 | (CH₂)₂CH₃ | —(CH₂)₄— | |
| 71 | CH(CH₃)₂ | —(CH₂)₄— | |
| 72 | (CH₂)₃CH₃ | —(CH₂)₄— | |
| 73 | CH₂CH(CH₃)₂ | —(CH₂)₄— | |
| 74 | C(CH₃)₃ | —(CH₂)₄— | |
| 75 | C₆H₅ | —(CH₂)₄— | |
| 76 | 2-F—C₆H₄ | —(CH₂)₄— | |
| 77 | 2-Cl—C₆H₄ | —(CH₂)₄— | |
| 78 | 2-CH₃—C₆H₄ | —(CH₂)₄— | |
| 79 | 2-CF₃—C₆H₄ | —(CH₂)₄— | |
| 80 | 2-OCH₃—C₆H₄ | —(CH₂)₄— | |
| 81 | 3-F—C₆H₄ | —(CH₂)₄— | |
| 82 | 3-Cl—C₆H₄ | —(CH₂)₄— | |
| 83 | 3-CH₃—C₆H₄ | —(CH₂)₄— | |
| 84 | 3-CF₃—C₆H₄ | —(CH₂)₄— | |
| 85 | 3-OCH₃—C₆H₄ | —(CH₂)₄— | |
| 86 | 4-F—C₆H₄ | —(CH₂)₄— | |
| 87 | 4-Cl—C₆H₄ | —(CH₂)₄— | |
| 88 | 4-CH₃—C₆H₄ | —(CH₂)₄— | |
| 89 | 4-CF₃—C₆H₄ | —(CH₂)₄— | |
| 90 | 4-OCH₃—C₆H₄ | —(CH₂)₄— | |
| 91 | 3,5-F₂—C₆H₃ | —(CH₂)₄— | |
| 92 | 3-F-5-Cl—C₆H₃ | —(CH₂)₄— | |
| 93 | 3-F-5-CH₃—C₆H₃ | —(CH₂)₄— | |
| 94 | 3-F-5-CF₃—C₆H₃ | —(CH₂)₄— | |
| 95 | 3-F-5-OCH₃—C₆H₃ | —(CH₂)₄— | |
| 96 | 3,5-Cl₂—C₆H₃ | —(CH₂)₄— | |
| 97 | 3-Cl-5-CH₃—C₆H₃ | —(CH₂)₄— | |
| 98 | 3-Cl-5-CF₃—C₆H₃ | —(CH₂)₄— | |
| 99 | 3-Cl-5-OCH₃—C₆H₃ | —(CH₂)₄— | |
| 100 | 3,5-(CH₃)₂—C₆H₃ | —(CH₂)₄— | |
| 101 | 3-CH₃-5-CF₃—C₆H₃ | —(CH₂)₄— | |
| 102 | 3-CH₃-5-OCH₃—C₆H₃ | —(CH₂)₄— | |
| 103 | 3,5-(CF₃)₂—C₆H₃ | —(CH₂)₄— | |
| 104 | 3-CF₃-5-OCH₃—C₆H₃ | —(CH₂)₄— | |
| 105 | 3,5-(OCH₃)₂—C₆H₃ | —(CH₂)₄— | |
| 106 | 2,4-F₂—C₆H₃ | —(CH₂)₄— | |
| 107 | 2-F-4-Cl—C₆H₃ | —(CH₂)₄— | |
| 108 | 2-F-4-CH₃—C₆H₃ | —(CH₂)₄— | |
| 109 | 2-F-4-CF₃—C₆H₃ | —(CH₂)₄— | |
| 110 | 2-F-4-OCH₃—C₆H₃ | —(CH₂)₄— | |
| 111 | 2-Cl-4-F—C₆H₃ | —(CH₂)₄— | |
| 112 | 2,4-Cl₂—C₆H₃ | —(CH₂)₄— | |
| 113 | 2-Cl-4-CH₃—C₆H₃ | —(CH₂)₄— | |
| 114 | 2-Cl-4-CF₃—C₆H₃ | —(CH₂)₄— | |
| 115 | 2-Cl-4-OCH₃—C₆H₃ | —(CH₂)₄— | |
| 116 | 2-CH₃-4-F—C₆H₃ | —(CH₂)₄— | |
| 117 | 2-CH₃-4-Cl—C₆H₃ | —(CH₂)₄— | |
| 118 | 2,4-(CH₃)₂—C₆H₃ | —(CH₂)₄— | |
| 119 | 2-CH₃-4-CF₃—C₆H₃ | —(CH₂)₄— | |
| 120 | 2-CH₃-4-OCH₃—C₆H₃ | —(CH₂)₄— | |
| 121 | 2-CF₃-4-F—C₆H₃ | —(CH₂)₄— | |
| 122 | 2-CF₃-4-Cl—C₆H₃ | —(CH₂)₄— | |
| 123 | 2-CF₃-4-CH₃—C₆H₃ | —(CH₂)₄— | |
| 124 | 2,4-(CF₃)₂—C₆H₃ | —(CH₂)₄— | |
| 125 | 2-CF₃-4-OCH₃—C₆H₃ | —(CH₂)₄— | |
| 126 | 2-OCH₃-4-F—C₆H₃ | —(CH₂)₄— | |
| 127 | 2-OCH₃-4-Cl—C₆H₃ | —(CH₂)₄— | |
| 128 | 2-OCH₃-4-CH₃—C₆H₃ | —(CH₂)₄— | |
| 129 | 2-OCH₃-4-CF₃—C₆H₃ | —(CH₂)₄— | |
| 130 | 2,4-(OCH₃)₂—C₆H₃ | —(CH₂)₄— | |
| 131 | F | —O—(CH₂)₂— | |
| 132 | Cl | —O—(CH₂)₂— | |
| 133 | CH₃ | —O—(CH₂)₂— | |
| 134 | C₂H₅ | —O—(CH₂)₂— | |
| 135 | (CH₂)₂CH₃ | —O—(CH₂)₂— | |
| 136 | CH(CH₃)₂ | —O—(CH₂)₂— | |
| 137 | (CH₂)₃CH₃ | —O—(CH₂)₂— | |
| 138 | CH₂CH(CH₃)₂ | —O—(CH₂)₂— | |
| 139 | C(CH₃)₃ | —O—(CH₂)₂— | |
| 140 | C₆H₅ | —O—(CH₂)₂— | |
| 141 | 2-F—C₆H₄ | —O—(CH₂)₂— | |
| 142 | 2-Cl—C₆H₄ | —O—(CH₂)₂— | |
| 143 | 2-CH₃—C₆H₄ | —O—(CH₂)₂— | |
| 144 | 2-CF₃—C₆H₄ | —O—(CH₂)₂— | |
| 145 | 2-OCH₃—C₆H₄ | —O—(CH₂)₂— | |
| 146 | 3-F—C₆H₄ | —O—(CH₂)₂— | |
| 147 | 3-Cl—C₆H₄ | —O—(CH₂)₂— | |
| 148 | 3-CH₃—C₆H₄ | —O—(CH₂)₂— | |
| 149 | 3-CF₃—C₆H₄ | —O—(CH₂)₂— | |
| 150 | 3-OCH₃—C₆H₄ | —O—(CH₂)₂— | |
| 151 | 4-F—C₆H₄ | —O—(CH₂)₂— | |
| 152 | 4-Cl—C₆H₄ | —O—(CH₂)₂— | |
| 153 | 4-CH₃—C₆H₄ | —O—(CH₂)₂— | |
| 154 | 4-CF₃—C₆H₄ | —O—(CH₂)₂— | |
| 155 | 4-OCH₃—C₆H₄ | —O—(CH₂)₂— | |
| 156 | 3,5-F₂—C₆H₃ | —O—(CH₂)₂— | |
| 157 | 3-F-5-Cl—C₆H₃ | —O—(CH₂)₂— | |
| 158 | 3-F-5-CH₃—C₆H₃ | —O—(CH₂)₂— | |
| 159 | 3-F-5-CF₃—C₆H₃ | —O—(CH₂)₂— | |
| 160 | 3-F-5-OCH₃—C₆H₃ | —O—(CH₂)₂— | |
| 161 | 3,5-Cl₂—C₆H₃ | —O—(CH₂)₂— | |
| 162 | 3-Cl-5-CH₃—C₆H₃ | —O—(CH₂)₂— | |
| 163 | 3-Cl-5-CF₃—C₆H₃ | —O—(CH₂)₂— | |
| 164 | 3-Cl-5-OCH₃—C₆H₃ | —O—(CH₂)₂— | |
| 165 | 3,5-(CH₃)₂—C₆H₃ | —O—(CH₂)₂— | |
| 166 | 3-CH₃-5-CF₃—C₆H₃ | —O—(CH₂)₂— | |
| 167 | 3-CH₃-5-OCH₃—C₆H₃ | —O—(CH₂)₂— | |
| 168 | 3,5-(CF₃)₂—C₆H₃ | —O—(CH₂)₂— | |
| 169 | 3-CF₃-5-OCH₃—C₆H₃ | —O—(CH₂)₂— | |
| 170 | 3,5-(OCH₃)₂—C₆H₃ | —O—(CH₂)₂— | |
| 171 | 2,4-F₂—C₆H₃ | —O—(CH₂)₂— | |
| 172 | 2-F-4-Cl—C₆H₃ | —O—(CH₂)₂— | |
| 173 | 2-F-4-CH₃—C₆H₃ | —O—(CH₂)₂— | |
| 174 | 2-F-4-CF₃—C₆H₃ | —O—(CH₂)₂— | |
| 175 | 2-F-4-OCH₃—C₆H₃ | —O—(CH₂)₂— | |
| 176 | 2-Cl-4-F—C₆H₃ | —O—(CH₂)₂— | |
| 177 | 2,4-Cl₂—C₆H₃ | —O—(CH₂)₂— | |
| 178 | 2-Cl-4-CH₃—C₆H₃ | —O—(CH₂)₂— | |
| 179 | 2-Cl-4-CF₃—C₆H₃ | —O—(CH₂)₂— | |
| 180 | 2-Cl-4-OCH₃—C₆H₃ | —O—(CH₂)₂— | |
| 181 | 2-CH₃-4-F—C₆H₃ | —O—(CH₂)₂— | |
| 182 | 2-CH₃-4-Cl—C₆H₃ | —O—(CH₂)₂— | |
| 183 | 2,4-(CH₃)₂—C₆H₃ | —O—(CH₂)₂— | |
| 184 | 2-CH₃-4-CF₃—C₆H₃ | —O—(CH₂)₂— | |
| 185 | 2-CH₃-4-OCH₃—C₆H₃ | —O—(CH₂)₂— | |
| 186 | 2-CF₃-4-F—C₆H₃ | —O—(CH₂)₂— | |
| 187 | 2-CF₃-4-Cl—C₆H₃ | —O—(CH₂)₂— | |
| 188 | 2-CF₃-4-CH₃—C₆H₃ | —O—(CH₂)₂— | |
| 189 | 2,4-(CF₃)₂—C₆H₃ | —O—(CH₂)₂— | |
| 190 | 2-CF₃-4-OCH₃—C₆H₃ | —O—(CH₂)₂— | |
| 191 | 2-OCH₃-4-F—C₆H₃ | —O—(CH₂)₂— | |
| 192 | 2-OCH₃-4-Cl—C₆H₃ | —O—(CH₂)₂— | |
| 193 | 2-OCH₃-4-CH₃—C₆H₃ | —O—(CH₂)₂— | |
| 194 | 2-OCH₃-4-CF₃—C₆H₃ | —O—(CH₂)₂— | |
| 195 | 2,4-(OCH₃)₂—C₆H₃ | —O—(CH₂)₂— | |
| 196 | F | —(CH₂)₂—O— | |
| 197 | Cl | —(CH₂)₂—O— | |
| 198 | CH₃ | —(CH₂)₂—O— | |
| 199 | C₂H₅ | —(CH₂)₂—O— | |
| 200 | (CH₂)₂CH₃ | —(CH₂)₂—O— | |
| 201 | CH(CH₃)₂ | —(CH₂)₂—O— | |
| 202 | (CH₂)₃CH₃ | —(CH₂)₂—O— | |
| 203 | CH₂CH(CH₃)₂ | —(CH₂)₂—O— | |
| 204 | C(CH₃)₃ | —(CH₂)₂—O— | |
| 205 | C₆H₅ | —(CH₂)₂—O— | |
| 206 | 2-F—C₆H₄ | —(CH₂)₂—O— | |
| 207 | 2-Cl—C₆H₄ | —(CH₂)₂—O— | |
| 208 | 2-CH₃—C₆H₄ | —(CH₂)₂—O— | |
| 209 | 2-CF₃—C₆H₄ | —(CH₂)₂—O— | |
| 210 | 2-OCH₃—C₆H₄ | —(CH₂)₂—O— | |
| 211 | 3-F—C₆H₄ | —(CH₂)₂—O— | |
| 212 | 3-Cl—C₆H₄ | —(CH₂)₂—O— | |
| 213 | 3-CH₃—C₆H₄ | —(CH₂)₂—O— | |
| 214 | 3-CF₃—C₆H₄ | —(CH₂)₂—O— | |
| 215 | 3-OCH₃—C₆H₄ | —(CH₂)₂—O— | |
| 216 | 4-F—C₆H₄ | —(CH₂)₂—O— | |
| 217 | 4-Cl—C₆H₄ | —(CH₂)₂—O— | |
| 218 | 4-CH₃—C₆H₄ | —(CH₂)₂—O— | |
| 219 | 4-CF₃—C₆H₄ | —(CH₂)₂—O— | |
| 220 | 4-OCH₃—C₆H₄ | —(CH₂)₂—O— | |
| 221 | 3,5-F₂—C₆H₃ | —(CH₂)₂—O— | |
| 222 | 3-F-5-Cl—C₆H₃ | —(CH₂)₂—O— | |
| 223 | 3-F-5-CH₃—C₆H₃ | —(CH₂)₂—O— | |
| 224 | 3-F-5-CF₃—C₆H₃ | —(CH₂)₂—O— | |
| 225 | 3-F-5-OCH₃—C₆H₃ | —(CH₂)₂—O— | |
| 226 | 3,5-Cl₂—C₆H₃ | —(CH₂)₂—O— | |
| 227 | 3-Cl-5-CH₃—C₆H₃ | —(CH₂)₂—O— | |
| 228 | 3-Cl-5-CF₃—C₆H₃ | —(CH₂)₂—O— | |
| 229 | 3-Cl-5-OCH₃—C₆H₃ | —(CH₂)₂—O— | |
| 230 | 3,5-(CH₃)₂—C₆H₃ | —(CH₂)₂—O— | |
| 231 | 3-CH₃-5-CF₃—C₆H₃ | —(CH₂)₂—O— | |
| 232 | 3-CH₃-5-OCH₃—C₆H₃ | —(CH₂)₂—O— | |
| 233 | 3,5-(CF₃)₂—C₆H₃ | —(CH₂)₂—O— | |
| 234 | 3-CF₃-5-OCH₃—C₆H₃ | —(CH₂)₂—O— | |
| 235 | 3,5-(OCH₃)₂—C₆H₃ | —(CH₂)₂—O— | |
| 236 | 2,4-F₂—C₆H₃ | —(CH₂)₂—O— | |
| 237 | 2-F-4-Cl—C₆H₃ | —(CH₂)₂—O— | |
| 238 | 2-F-4-CH₃—C₆H₃ | —(CH₂)₂—O— | |
| 239 | 2-F-4-CF₃—C₆H₃ | —(CH₂)₂—O— | |
| 240 | 2-F-4-OCH₃—C₆H₃ | —(CH₂)₂—O— | |
| 241 | 2-Cl-4-F—C₆H₃ | —(CH₂)₂—O— | |
| 242 | 2,4-Cl₂—C₆H₃ | —(CH₂)₂—O— | |
| 243 | 2-Cl-4-CH₃—C₆H₃ | —(CH₂)₂—O— | |
| 244 | 2-Cl-4-CF₃—C₆H₃ | —(CH₂)₂—O— | |
| 245 | 2-Cl-4-OCH₃—C₆H₃ | —(CH₂)₂—O— | |
| 246 | 2-CH₃-4-F—C₆H₃ | —(CH₂)₂—O— | |
| 247 | 2-CH₃-4-Cl—C₆H₃ | —(CH₂)₂—O— | |
| 248 | 2,4-(CH₃)₂—C₆H₃ | —(CH₂)₂—O— | |
| 249 | 2-CH₃-4-CF₃—C₆H₃ | —(CH₂)₂—O— | |
| 250 | 2-CH₃-4-OCH₃—C₆H₃ | —(CH₂)₂—O— | |
| 251 | 2-CF₃-4-F—C₆H₃ | —(CH₂)₂—O— | |
| 252 | 2-CF₃-4-Cl—C₆H₃ | —(CH₂)₂—O— | |
| 253 | 2-CF₃-4-CH₃—C₆H₃ | —(CH₂)₂—O— | |
| 254 | 2,4-(CF₃)₂—C₆H₃ | —(CH₂)₂—O— | |
| 255 | 2-CF₃-4-OCH₃—C₆H₃ | —(CH₂)₂—O— | |
| 256 | 2-OCH₃-4-F—C₆H₃ | —(CH₂)₂—O— | |
| 257 | 2-OCH₃-4-Cl—C₆H₃ | —(CH₂)₂—O— | |
| 258 | 2-OCH₃-4-CH₃—C₆H₃ | —(CH₂)₂—O— | |
| 259 | 2-OCH₃-4-CF₃—C₆H₃ | —(CH₂)₂—O— | |
| 260 | 2,4-(OCH₃)₂—C₆H₃ | —(CH₂)₂—O— | |
| 261 | F | —O—(CH₂)₃— | |
| 262 | Cl | —O—(CH₂)₃— | |
| 263 | CH₃ | —O—(CH₂)₃— | |
| 264 | C₂H₅ | —O—(CH₂)₃— | |
| 265 | (CH₂)₂CH₃ | —O—(CH₂)₃— | |
| 266 | CH(CH₃)₂ | —O—(CH₂)₃— | |
| 267 | (CH₂)₃CH₃ | —O—(CH₂)₃— | |
| 268 | CH₂CH(CH₃)₂ | —O—(CH₂)₃— | |
| 269 | C(CH₃)₃ | —O—(CH₂)₃— | |
| 270 | C₆H₅ | —O—(CH₂)₃— | |
| 271 | 2-F—C₆H₄ | —O—(CH₂)₃— | |
| 272 | 2-Cl—C₆H₄ | —O—(CH₂)₃— | |
| 273 | 2-CH₃—C₆H₄ | —O—(CH₂)₃— | |
| 274 | 2-CF₃—C₆H₄ | —O—(CH₂)₃— | |
| 275 | 2-OCH₃—C₆H₄ | —O—(CH₂)₃— | |
| 276 | 3-F—C₆H₄ | —O—(CH₂)₃— | |
| 277 | 3-Cl—C₆H₄ | —O—(CH₂)₃— | |
| 278 | 3-CH₃—C₆H₄ | —O—(CH₂)₃— | |
| 279 | 3-CF₃—C₆H₄ | —O—(CH₂)₃— | |
| 280 | 3-OCH₃—C₆H₄ | —O—(CH₂)₃— | |
| 281 | 4-F—C₆H₄ | —O—(CH₂)₃— | |
| 282 | 4-Cl—C₆H₄ | —O—(CH₂)₃— | |
| 283 | 4-CH₃—C₆H₄ | —O—(CH₂)₃— | |
| 284 | 4-CF₃—C₆H₄ | —O—(CH₂)₃— | |
| 285 | 4-OCH₃—C₆H₄ | —O—(CH₂)₃— | |
| 286 | 3,5-F₂—C₆H₃ | —O—(CH₂)₃— | |
| 287 | 3-F-5-Cl—C₆H₃ | —O—(CH₂)₃— | |
| 288 | 3-F-5-CH₃—C₆H₃ | —O—(CH₂)₃— | |
| 289 | 3-F-5-CF₃—C₆H₃ | —O—(CH₂)₃— | |
| 290 | 3-F-5-OCH₃—C₆H₃ | —O—(CH₂)₃— | |
| 291 | 3,5-Cl₂—C₆H₃ | —O—(CH₂)₃— | |
| 292 | 3-Cl-5-CH₃—C₆H₃ | —O—(CH₂)₃— | |
| 293 | 3-Cl-5-CF₃—C₆H₃ | —O—(CH₂)₃— | |
| 294 | 3-Cl-5-OCH₃—C₆H₃ | —O—(CH₂)₃— | |
| 295 | 3,5-(CH₃)₂—C₆H₃ | —O—(CH₂)₃— | |
| 296 | 3-CH₃-5-CF₃—C₆H₃ | —O—(CH₂)₃— | |
| 297 | 3-CH₃-5-OCH₃—C₆H₃ | —O—(CH₂)₃— | |
| 298 | 3,5-(CF₃)₂—C₆H₃ | —O—(CH₂)₃— | |
| 299 | 3-CF₃-5-OCH₃—C₆H₃ | —O—(CH₂)₃— | |
| 300 | 3,5-(OCH₃)₂—C₆H₃ | —O—(CH₂)₃— | |
| 301 | 2,4-F₂—C₆H₃ | —O—(CH₂)₃— | |
| 302 | 2-F-4-Cl—C₆H₃ | —O—(CH₂)₃— | |
| 303 | 2-F-4-CH₃—C₆H₃ | —O—(CH₂)₃— | |
| 304 | 2-F-4-CF₃—C₆H₃ | —O—(CH₂)₃— | |
| 305 | 2-F-4-OCH₃—C₆H₃ | —O—(CH₂)₃— | |
| 306 | 2-Cl-4-F—C₆H₃ | —O—(CH₂)₃— | |
| 307 | 2,4-Cl₂—C₆H₃ | —O—(CH₂)₃— | |
| 308 | 2-Cl-4-CH₃—C₆H₃ | —O—(CH₂)₃— | |
| 309 | 2-Cl-4-CF₃—C₆H₃ | —O—(CH₂)₃— | |
| 310 | 2-Cl-4-OCH₃—C₆H₃ | —O—(CH₂)₃— | |
| 311 | 2-CH₃-4-F—C₆H₃ | —O—(CH₂)₃— | |
| 312 | 2-CH₃-4-Cl—C₆H₃ | —O—(CH₂)₃— | |
| 313 | 2,4-(CH₃)₂—C₆H₃ | —O—(CH₂)₃— | |
| 314 | 2-CH₃-4-CF₃—C₆H₃ | —O—(CH₂)₃— | |
| 315 | 2-CH₃-4-OCH₃—C₆H₃ | —O—(CH₂)₃— | |
| 316 | 2-CF₃-4-F—C₆H₃ | —O—(CH₂)₃— | |
| 317 | 2-CF₃-4-Cl—C₆H₃ | —O—(CH₂)₃— | |
| 318 | 2-CF₃-4-CH₃—C₆H₃ | —O—(CH₂)₃— | |
| 319 | 2,4-(CF₃)₂—C₆H₃ | —O—(CH₂)₃— | |
| 320 | 2-CF₃-4-OCH₃—C₆H₃ | —O—(CH₂)₃— | |
| 321 | 2-OCH₃-4-F—C₆H₃ | —O—(CH₂)₃— | |
| 322 | 2-OCH₃-4-Cl—C₆H₃ | —O—(CH₂)₃— | |
| 323 | 2-OCH₃-4-CH₃—C₆H₃ | —O—(CH₂)₃— | |
| 324 | 2-OCH₃-4-CF₃—C₆H₃ | —O—(CH₂)₃— | |
| 325 | 2,4-(OCH₃)₂—C₆H₃ | —O—(CH₂)₃— | |
| 326 | F | —(CH₂)₃—O— | |
| 327 | Cl | —(CH₂)₃—O— | |
| 328 | CH₃ | —(CH₂)₃—O— | |
| 329 | C₂H₅ | —(CH₂)₃—O— | |
| 330 | (CH₂)₂CH₃ | —(CH₂)₃—O— | |
| 331 | CH(CH₃)₂ | —(CH₂)₃—O— | |
| 332 | (CH₂)₃CH₃ | —(CH₂)₃—O— | |
| 333 | CH₂CH(CH₃)₂ | —(CH₂)₃—O— | |
| 334 | C(CH₃)₃ | —(CH₂)₃—O— | |
| 335 | C₆H₅ | —(CH₂)₃—O— | |
| 336 | 2-F—C₆H₄ | —(CH₂)₃—O— | |
| 337 | 2-Cl—C₆H₄ | —(CH₂)₃—O— | |
| 338 | 2-CH₃—C₆H₄ | —(CH₂)₃—O— | |
| 339 | 2-CF₃—C₆H₄ | —(CH₂)₃—O— | |
| 340 | 2-OCH₃—C₆H₄ | —(CH₂)₃—O— | |
| 341 | 3-F—C₆H₄ | —(CH₂)₃—O— | |
| 342 | 3-Cl—C₆H₄ | —(CH₂)₃—O— | |
| 343 | 3-CH₃—C₆H₄ | —(CH₂)₃—O— | |
| 344 | 3-CF₃—C₆H₄ | —(CH₂)₃—O— | |
| 345 | 3-OCH₃—C₆H₄ | —(CH₂)₃—O— | |
| 346 | 4-F—C₆H₄ | —(CH₂)₃—O— | |
| 347 | 4-Cl—C₆H₄ | —(CH₂)₃—O— | |
| 348 | 4-CH₃—C₆H₄ | —(CH₂)₃—O— | |
| 349 | 4-CF₃—C₆H₄ | —(CH₂)₃—O— | |
| 350 | 4-OCH₃—C₆H₄ | —(CH₂)₃—O— | |
| 351 | 3,5-F₂—C₆H₃ | —(CH₂)₃—O— | |
| 352 | 3-F-5-Cl—C₆H₃ | —(CH₂)₃—O— | |
| 353 | 3-F-5-CH₃—C₆H₃ | —(CH₂)₃—O— | |
| 354 | 3-F-5-CF₃—C₆H₃ | —(CH₂)₃—O— | |
| 355 | 3-F-5-OCH₃—C₆H₃ | —(CH₂)₃—O— | |
| 356 | 3,5-Cl₂—C₆H₃ | —(CH₂)₃—O— | |
| 357 | 3-Cl-5-CH₃—C₆H₃ | —(CH₂)₃—O— | |
| 358 | 3-Cl-5-CF₃—C₆H₃ | —(CH₂)₃—O— | |
| 359 | 3-Cl-5-OCH₃—C₆H₃ | —(CH₂)₃—O— | |
| 360 | 3,5-(CH₃)₂—C₆H₃ | —(CH₂)₃—O— | |
| 361 | 3-CH₃-5-CF₃—C₆H₃ | —(CH₂)₃—O— | |
| 362 | 3-CH₃-5-OCH₃—C₆H₃ | —(CH₂)₃—O— | |
| 363 | 3,5-(CF₃)₂—C₆H₃ | —(CH₂)₃—O— | |
| 364 | 3-CF₃-5-OCH₃—C₆H₃ | —(CH₂)₃—O— | |
| 365 | 3,5-(OCH₃)₂—C₆H₃ | —(CH₂)₃—O— | |
| 366 | 2,4-F₂—C₆H₃ | —(CH₂)₃—O— | |
| 367 | 2-F-4-Cl—C₆H₃ | —(CH₂)₃—O— | |
| 368 | 2-F-4-CH₃—C₆H₃ | —(CH₂)₃—O— | |
| 369 | 2-F-4-CF₃—C₆H₃ | —(CH₂)₃—O— | |
| 370 | 2-F-4-OCH₃—C₆H₃ | —(CH₂)₃—O— | |
| 371 | 2-Cl-4-F—C₆H₃ | —(CH₂)₃—O— | |
| 372 | 2,4-Cl₂—C₆H₃ | —(CH₂)₃—O— | |
| 373 | 2-Cl-4-CH₃—C₆H₃ | —(CH₂)₃—O— | |
| 374 | 2-Cl-4-CF₃—C₆H₃ | —(CH₂)₃—O— | |
| 375 | 2-Cl-4-OCH₃—C₆H₃ | —(CH₂)₃—O— | |
| 376 | 2-CH₃-4-F—C₆H₃ | —(CH₂)₃—O— | |
| 377 | 2-CH₃-4-Cl—C₆H₃ | —(CH₂)₃—O— | |
| 378 | 2,4-(CH₃)₂—C₆H₃ | —(CH₂)₃—O— | |
| 379 | 2-CH₃-4-CF₃—C₆H₃ | —(CH₂)₃—O— | |
| 380 | 2-CH₃-4-OCH₃—C₆H₃ | —(CH₂)₃—O— | |
| 381 | 2-CF₃-4-F—C₆H₃ | —(CH₂)₃—O— | |
| 382 | 2-CF₃-4-Cl—C₆H₃ | —(CH₂)₃—O— | |
| 383 | 2-CF₃-4-CH₃—C₆H₃ | —(CH₂)₃—O— | |
| 384 | 2,4-(CF₃)₂—C₆H₃ | —(CH₂)₃—O— | |
| 385 | 2-CF₃-4-OCH₃—C₆H₃ | —(CH₂)₃—O— | |
| 386 | 2-OCH₃-4-F—C₆H₃ | —(CH₂)₃—O— | |
| 387 | 2-OCH₃-4-Cl—C₆H₃ | —(CH₂)₃—O— | |
| 388 | 2-OCH₃-4-CH₃—C₆H₃ | —(CH₂)₃—O— | |
| 389 | 2-OCH₃-4-CF₃—C₆H₃ | —(CH₂)₃—O— | |
| 390 | 2,4-(OCH₃)₂—C₆H₃ | —(CH₂)₃—O— | |
| 391 | F | —CH═CH—CH═CH— | |
| 392 | Cl | —CH═CH—CH═CH— | |
| 393 | CH₃ | —CH═CH—CH═CH— | |
| 394 | C₂H₅ | —CH═CH—CH═CH— | |
| 395 | (CH₂)₂CH₃ | —CH═CH—CH═CH— | |
| 396 | CH(CH₃)₂ | —CH═CH—CH═CH— | |
| 397 | (CH₂)₃CH₃ | —CH═CH—CH═CH— | |
| 398 | CH₂CH(CH₃)₂ | —CH═CH—CH═CH— | |
| 399 | C(CH₃)₃ | —CH═CH—CH═CH— | |
| 400 | C₆H₅ | —CH═CH—CH═CH— | |
| 401 | 2-F—C₆H₄ | —CH═CH—CH═CH— | |
| 402 | 2-Cl—C₆H₄ | —CH═CH—CH═CH— | |
| 403 | 2-CH₃—C₆H₄ | —CH═CH—CH═CH— | |
| 404 | 2-CF₃—C₆H₄ | —CH═CH—CH═CH— | |
| 405 | 2-OCH₃—C₆H₄ | —CH═CH—CH═CH— | |
| 406 | 3-F—C₆H₄ | —CH═CH—CH═CH— | |
| 407 | 3-Cl—C₆H₄ | —CH═CH—CH═CH— | |
| 408 | 3-CH₃—C₆H₄ | —CH═CH—CH═CH— | |
| 409 | 3-CF₃—C₆H₄ | —CH═CH—CH═CH— | |
| 410 | 3-OCH₃—C₆H₄ | —CH═CH—CH═CH— | |
| 411 | 4-F—C₆H₄ | —CH═CH—CH═CH— | |
| 412 | 4-Cl—C₆H₄ | —CH═CH—CH═CH— | |
| 413 | 4-CH₃—C₆H₄ | —CH═CH—CH═CH— | |
| 414 | 4-CF₃—C₆H₄ | —CH═CH—CH═CH— | |
| 415 | 4-OCH₃—C₆H₄ | —CH═CH—CH═CH— | |
| 416 | 3,5-F₂—C₆H₃ | —CH═CH—CH═CH— | |
| 417 | 3-F-5-Cl—C₆H₃ | —CH═CH—CH═CH— | |
| 418 | 3-F-5-CH₃—C₆H₃ | —CH═CH—CH═CH— | |
| 419 | 3-F-5-CF₃—C₆H₃ | —CH═CH—CH═CH— | |
| 420 | 3-F-5-OCH₃—C₆H₃ | —CH═CH—CH═CH— | |
| 421 | 3,5-Cl₂—C₆H₃ | —CH═CH—CH═CH— | |
| 422 | 3-Cl-5-CH₃—C₆H₃ | —CH═CH—CH═CH— | |
| 423 | 3-Cl-5-CF₃—C₆H₃ | —CH═CH—CH═CH— | |
| 424 | 3-Cl-5-OCH₃—C₆H₃ | —CH═CH—CH═CH— | |
| 425 | 3,5-(CH₃)₂—C₆H₃ | —CH═CH—CH═CH— | |
| 426 | 3-CH₃-5-CF₃—C₆H₃ | —CH═CH—CH═CH— | |
| 427 | 3-CH₃-5-OCH₃—C₆H₃ | —CH═CH—CH═CH— | |
| 428 | 3,5-(CF₃)₂—C₆H₃ | —CH═CH—CH═CH— | |
| 429 | 3-CF₃-5-OCH₃—C₆H₃ | —CH═CH—CH═CH— | |
| 430 | 3,5-(OCH₃)₂—C₆H₃ | —CH═CH—CH═CH— | |
| 431 | 2,4-F₂—C₆H₃ | —CH═CH—CH═CH— | |
| 432 | 2-F-4-Cl—C₆H₃ | —CH═CH—CH═CH— | |
| 433 | 2-F-4-CH₃—C₆H₃ | —CH═CH—CH═CH— | |
| 434 | 2-F-4-CF₃—C₆H₃ | —CH═CH—CH═CH— | |
| 435 | 2-F-4-OCH₃—C₆H₃ | —CH═CH—CH═CH— | |
| 436 | 2-Cl-4-F—C₆H₃ | —CH═CH—CH═CH— | |
| 437 | 2,4-Cl₂—C₆H₃ | —CH═CH—CH═CH— | |
| 438 | 2-Cl-4-CH₃—C₆H₃ | —CH═CH—CH═CH— | |
| 439 | 2-Cl-4-CF₃—C₆H₃ | —CH═CH—CH═CH— | |
| 440 | 2-Cl-4-OCH₃—C₆H₃ | —CH═CH—CH═CH— | |
| 441 | 2-CH₃-4-F—C₆H₃ | —CH═CH—CH═CH— | |
| 442 | 2-CH₃-4-Cl—C₆H₃ | —CH═CH—CH═CH— | |
| 443 | 2,4-(CH₃)₂—C₆H₃ | —CH═CH—CH═CH— | |
| 444 | 2-CH₃-4-CF₃—C₆H₃ | —CH═CH—CH═CH— | |
| 445 | 2-CH₃-4-OCH₃—C₆H₃ | —CH═CH—CH═CH— | |
| 446 | 2-CF₃-4-F—C₆H₃ | —CH═CH—CH═CH— | |
| 447 | 2-CF₃-4-Cl—C₆H₃ | —CH═CH—CH═CH— | |
| 448 | 2-CF₃-4-CH₃—C₆H₃ | —CH═CH—CH═CH— | |
| 449 | 2,4-(CF₃)₂—C₆H₃ | —CH═CH—CH═CH— | |
| 450 | 2-CF₃-4-OCH₃—C₆H₃ | —CH═CH—CH═CH— | |
| 451 | 2-OCH₃-4-F—C₆H₃ | —CH═CH—CH═CH— | |
| 452 | 2-OCH₃-4-Cl—C₆H₃ | —CH═CH—CH═CH— | |
| 453 | 2-OCH₃-4-CH₃—C₆H₃ | —CH═CH—CH═CH— | |
| 454 | 2-OCH₃-4-CF₃—C₆H₃ | —CH═CH—CH═CH— | |
| 455 | 2,4-(OCH₃)₂—C₆H₃ | —CH═CH—CH═CH— | |
| 456 | F | —O—(CH₂)₂—O— | |
| 457 | Cl | —O—(CH₂)₂—O— | |
| 458 | CH₃ | —O—(CH₂)₂—O— | |
| 459 | C₂H₅ | —O—(CH₂)₂—O— | |
| 460 | (CH₂)₂CH₃ | —O—(CH₂)₂—O— | |
| 461 | CH(CH₃)₂ | —O—(CH₂)₂—O— | |
| 462 | (CH₂)₃CH₃ | —O—(CH₂)₂—O— | |
| 463 | CH₂CH(CH₃)₂ | —O—(CH₂)₂—O— | |
| 464 | C(CH₃)₃ | —O—(CH₂)₂—O— | |
| 465 | C₆H₅ | —O—(CH₂)₂—O— | |
| 466 | 2-F—C₆H₄ | —O—(CH₂)₂—O— | |
| 467 | 2-Cl—C₆H₄ | —O—(CH₂)₂—O— | |
| 468 | 2-CH₃—C₆H₄ | —O—(CH₂)₂—O— | |
| 469 | 2-CF₃—C₆H₄ | —O—(CH₂)₂—O— | |
| 470 | 2-OCH₃—C₆H₄ | —O—(CH₂)₂—O— | |
| 471 | 3-F—C₆H₄ | —O—(CH₂)₂—O— | |
| 472 | 3-Cl—C₆H₄ | —O—(CH₂)₂—O— | |
| 473 | 3-CH₃—C₆H₄ | —O—(CH₂)₂—O— | |
| 474 | 3-CF₃—C₆H₄ | —O—(CH₂)₂—O— | |
| 475 | 3-OCH₃—C₆H₄ | —O—(CH₂)₂—O— | |
| 476 | 4-F—C₆H₄ | —O—(CH₂)₂—O— | |
| 477 | 4-Cl—C₆H₄ | —O—(CH₂)₂—O— | |
| 478 | 4-CH₃—C₆H₄ | —O—(CH₂)₂—O— | |
| 479 | 4-CF₃—C₆H₄ | —O—(CH₂)₂—O— | |
| 480 | 4-OCH₃—C₆H₄ | —O—(CH₂)₂—O— | |
| 481 | 3,5-F₂—C₆H₃ | —O—(CH₂)₂—O— | |
| 482 | 3-F-5-Cl—C₆H₃ | —O—(CH₂)₂—O— | |
| 483 | 3-F-5-CH₃—C₆H₃ | —O—(CH₂)₂—O— | |
| 484 | 3-F-5-CF₃—C₆H₃ | —O—(CH₂)₂—O— | |
| 485 | 3-F-5-OCH₃—C₆H₃ | —O—(CH₂)₂—O— | |
| 486 | 3,5-Cl₂—C₆H₃ | —O—(CH₂)₂—O— | |
| 487 | 3-Cl-5-CH₃—C₆H₃ | —O—(CH₂)₂—O— | |
| 488 | 3-Cl-5-CF₃—C₆H₃ | —O—(CH₂)₂—O— | |
| 489 | 3-Cl-5-OCH₃—C₆H₃ | —O—(CH₂)₂—O— | |
| 490 | 3,5-(CH₃)₂—C₆H₃ | —O—(CH₂)₂—O— | |
| 491 | 3-CH₃-5-CF₃—C₆H₃ | —O—(CH₂)₂—O— | |
| 492 | 3-CH₃-5-OCH₃—C₆H₃ | —O—(CH₂)₂—O— | |
| 493 | 3,5-(CF₃)₂—C₆H₃ | —O—(CH₂)₂—O— | |
| 494 | 3-CF₃-5-OCH₃—C₆H₃ | —O—(CH₂)₂—O— | |
| 495 | 3,5-(OCH₃)₂—C₆H₃ | —O—(CH₂)₂—O— | |
| 496 | 2,4-F₂—C₆H₃ | —O—(CH₂)₂—O— | |
| 497 | 2-F-4-Cl—C₆H₃ | —O—(CH₂)₂—O— | |
| 498 | 2-F-4-CH₃—C₆H₃ | —O—(CH₂)₂—O— | |
| 499 | 2-F-4-CF₃—C₆H₃ | —O—(CH₂)₂—O— | |
| 500 | 2-F-4-OCH₃—C₆H₃ | —O—(CH₂)₂—O— | |
| 501 | 2-Cl-4-F—C₆H₃ | —O—(CH₂)₂—O— | |
| 502 | 2,4-Cl₂—C₆H₃ | —O—(CH₂)₂—O— | |
| 503 | 2-Cl-4-CH₃—C₆H₃ | —O—(CH₂)₂—O— | |
| 504 | 2-Cl-4-CF₃—C₆H₃ | —O—(CH₂)₂—O— | |
| 505 | 2-Cl-4-OCH₃—C₆H₃ | —O—(CH₂)₂—O— | |
| 506 | 2-CH₃-4-F—C₆H₃ | —O—(CH₂)₂—O— | |
| 507 | 2-CH₃-4-Cl—C₆H₃ | —O—(CH₂)₂—O— | |
| 508 | 2,4-(CH₃)₂—C₆H₃ | —O—(CH₂)₂—O— | |
| 509 | 2-CH₃-4-CF₃—C₆H₃ | —O—(CH₂)₂—O— | |
| 510 | 2-CH₃-4-OCH₃—C₆H₃ | —O—(CH₂)₂—O— | |
| 511 | 2-CF₃-4-F—C₆H₃ | —O—(CH₂)₂—O— | |
| 512 | 2-CF₃-4-Cl—C₆H₃ | —O—(CH₂)₂—O— | |
| 513 | 2-CF₃-4-CH₃—C₆H₃ | —O—(CH₂)₂—O— | |
| 514 | 2,4-(CF₃)₂—C₆H₃ | —O—(CH₂)₂—O— | |
| 515 | 2-CF₃-4-OCH₃—C₆H₃ | —O—(CH₂)₂—O— | |
| 516 | 2-OCH₃-4-F—C₆H₃ | —O—(CH₂)₂—O— | |
| 517 | 2-OCH₃-4-Cl—C₆H₃ | —O—(CH₂)₂—O— | |
| 518 | 2-OCH₃-4-CH₃—C₆H₃ | —O—(CH₂)₂—O— | |
| 519 | 2-OCH₃-4-CF₃—C₆H₃ | —O—(CH₂)₂—O— | |
| 520 | 2,4-(OCH₃)₂—C₆H₃ | —O—(CH₂)₂—O— | |
| 521 | F | —O—(CH₂)₃—O— | |
| 522 | Cl | —O—(CH₂)₃—O— | |
| 523 | CH₃ | —O—(CH₂)₃—O— | |
| 524 | C₂H₅ | —O—(CH₂)₃—O— | |
| 525 | (CH₂)₂CH₃ | —O—(CH₂)₃—O— | |
| 526 | CH(CH₃)₂ | —O—(CH₂)₃—O— | |
| 527 | (CH₂)₃CH₃ | —O—(CH₂)₃—O— | |
| 528 | CH₂CH(CH₃)₂ | —O—(CH₂)₃—O— | |
| 529 | C(CH₃)₃ | —O—(CH₂)₃—O— | |
| 530 | C₆H₅ | —O—(CH₂)₃—O— | |
| 531 | 2-F—C₆H₄ | —O—(CH₂)₃—O— | |
| 532 | 2-Cl—C₆H₄ | —O—(CH₂)₃—O— | |
| 533 | 2-CH₃—C₆H₄ | —O—(CH₂)₃—O— | |
| 534 | 2-CF₃—C₆H₄ | —O—(CH₂)₃—O— | |
| 535 | 2-OCH₃—C₆H₄ | —O—(CH₂)₃—O— | |
| 536 | 3-F—C₆H₄ | —O—(CH₂)₃—O— | |
| 537 | 3-Cl—C₆H₄ | —O—(CH₂)₃—O— | |
| 538 | 3-CH₃—C₆H₄ | —O—(CH₂)₃—O— | |
| 539 | 3-CF₃—C₆H₄ | —O—(CH₂)₃—O— | |
| 540 | 3-OCH₃—C₆H₄ | —O—(CH₂)₃—O— | |
| 541 | 4-F—C₆H₄ | —O—(CH₂)₃—O— | |
| 542 | 4-Cl—C₆H₄ | —O—(CH₂)₃—O— | |
| 543 | 4-CH₃—C₆H₄ | —O—(CH₂)₃—O— | |
| 544 | 4-CF₃—C₆H₄ | —O—(CH₂)₃—O— | |
| 545 | 4-OCH₃—C₆H₄ | —O—(CH₂)₃—O— | |
| 546 | 3,5-F₂—C₆H₃ | —O—(CH₂)₃—O— | |
| 547 | 3-F-5-Cl—C₆H₃ | —O—(CH₂)₃—O— | |
| 548 | 3-F-5-CH₃—C₆H₃ | —O—(CH₂)₃—O— | |
| 549 | 3-F-5-CF₃—C₆H₃ | —O—(CH₂)₃—O— | |
| 550 | 3-F-5-OCH₃—C₆H₃ | —O—(CH₂)₃—O— | |
| 551 | 3,5-Cl₂—C₆H₃ | —O—(CH₂)₃—O— | |
| 552 | 3-Cl-5-CH₃—C₆H₃ | —O—(CH₂)₃—O— | |
| 553 | 3-Cl-5-CF₃—C₆H₃ | —O—(CH₂)₃—O— | |
| 554 | 3-Cl-5-OCH₃—C₆H₃ | —O—(CH₂)₃—O— | |
| 555 | 3,5-(CH₃)₂—C₆H₃ | —O—(CH₂)₃—O— | |
| 556 | 3-CH₃-5-CF₃—C₆H₃ | —O—(CH₂)₃—O— | |
| 557 | 3-CH₃-5-OCH₃—C₆H₃ | —O—(CH₂)₃—O— | |
| 558 | 3,5-(CF₃)₂—C₆H₃ | —O—(CH₂)₃—O— | |
| 559 | 3-CF₃-5-OCH₃—C₆H₃ | —O—(CH₂)₃—O— | |
| 560 | 3,5-(OCH₃)₂—C₆H₃ | —O—(CH₂)₃—O— | |
| 561 | 2,4-F₂—C₆H₃ | —O—(CH₂)₃—O— | |
| 562 | 2-F-4-Cl—C₆H₃ | —O—(CH₂)₃—O— | |
| 563 | 2-F-4-CH₃—C₆H₃ | —O—(CH₂)₃—O— | |
| 564 | 2-F-4-CF₃—C₆H₃ | —O—(CH₂)₃—O— | |
| 565 | 2-F-4-OCH₃—C₆H₃ | —O—(CH₂)₃—O— | |
| 566 | 2-Cl-4-F—C₆H₃ | —O—(CH₂)₃—O— | |
| 567 | 2,4-Cl₂—C₆H₃ | —O—(CH₂)₃—O— | |
| 568 | 2-Cl-4-CH₃—C₆H₃ | —O—(CH₂)₃—O— | |
| 569 | 2-Cl-4-CF₃—C₆H₃ | —O—(CH₂)₃—O— | |
| 570 | 2-Cl-4-OCH₃—C₆H₃ | —O—(CH₂)₃—O— | |
| 571 | 2-CH₃-4-F—C₆H₃ | —O—(CH₂)₃—O— | |
| 572 | 2-CH₃-4-Cl—C₆H₃ | —O—(CH₂)₃—O— | |
| 573 | 2,4-(CH₃)₂—C₆H₃ | —O—(CH₂)₃—O— | |
| 574 | 2-CH₃-4-CF₃—C₆H₃ | —O—(CH₂)₃—O— | |
| 575 | 2-CH₃-4-OCH₃—C₆H₃ | —O—(CH₂)₃—O— | |
| 576 | 2-CF₃-4-F—C₆H₃ | —O—(CH₂)₃—O— | |
| 577 | 2-CF₃-4-Cl—C₆H₃ | —O—(CH₂)₃—O— | |
| 578 | 2-CF₃-4-CH₃—C₆H₃ | —O—(CH₂)₃—O— | |
| 579 | 2,4-(CF₃)₂—C₆H₃ | —O—(CH₂)₃—O— | |
| 580 | 2-CF₃-4-OCH₃—C₆H₃ | —O—(CH₂)₃—O— | |
| 581 | 2-OCH₃-4-F—C₆H₃ | —O—(CH₂)₃—O— | |
| 582 | 2-OCH₃-4-Cl—C₆H₃ | —O—(CH₂)₃—O— | |
| 583 | 2-OCH₃-4-CH₃—C₆H₃ | —O—(CH₂)₃—O— | |
| 584 | 2-OCH₃-4-CF₃—C₆H₃ | —O—(CH₂)₃—O— | |
| 585 | 2,4-(OCH₃)₂—C₆H₃ | —O—(CH₂)₃—O— | |
| 586 | F | —O—(CF₂)₂—O— | |
| 587 | Cl | —O—(CF₂)₂—O— | |
| 588 | CH₃ | —O—(CF₂)₂—O— | |
| 589 | C₂H₅ | —O—(CF₂)₂—O— | |
| 590 | (CH₂)₂CH₃ | —O—(CF₂)₂—O— | |
| 591 | CH(CH₃)₂ | —O—(CF₂)₂—O— | |
| 592 | (CH₂)₃CH₃ | —O—(CF₂)₂—O— | |
| 593 | CH₂CH(CH₃)₂ | —O—(CF₂)₂—O— | |
| 594 | C(CH₃)₃ | —O—(CF₂)₂—O— | |
| 595 | C₆H₅ | —O—(CF₂)₂—O— | |
| 596 | 2-F—C₆H₄ | —O—(CF₂)₂—O— | |
| 597 | 2-Cl—C₆H₄ | —O—(CF₂)₂—O— | |
| 598 | 2-CH₃—C₆H₄ | —O—(CF₂)₂—O— | |
| 599 | 2-CF₃—C₆H₄ | —O—(CF₂)₂—O— | |
| 600 | 2-OCH₃—C₆H₄ | —O—(CF₂)₂—O— | |
| 601 | 3-F—C₆H₄ | —O—(CF₂)₂—O— | |
| 602 | 3-Cl—C₆H₄ | —O—(CF₂)₂—O— | |
| 603 | 3-CH₃—C₆H₄ | —O—(CF₂)₂—O— | |
| 604 | 3-CF₃—C₆H₄ | —O—(CF₂)₂—O— | |
| 605 | 3-OCH₃—C₆H₄ | —O—(CF₂)₂—O— | |
| 606 | 4-F—C₆H₄ | —O—(CF₂)₂—O— | |
| 607 | 4-Cl—C₆H₄ | —O—(CF₂)₂—O— | |
| 608 | 4-CH₃—C₆H₄ | —O—(CF₂)₂—O— | |
| 609 | 4-CF₃—C₆H₄ | —O—(CF₂)₂—O— | |
| 610 | 4-OCH₃—C₆H₄ | —O—(CF₂)₂—O— | |
| 611 | 3,5-F₂—C₆H₃ | —O—(CF₂)₂—O— | |
| 612 | 3-F-5-Cl—C₆H₃ | —O—(CF₂)₂—O— | |
| 613 | 3-F-5-CH₃—C₆H₃ | —O—(CF₂)₂—O— | |
| 614 | 3-F-5-CF₃—C₆H₃ | —O—(CF₂)₂—O— | |
| 615 | 3-F-5-OCH₃—C₆H₃ | —O—(CF₂)₂—O— | |
| 616 | 3,5-Cl₂—C₆H₃ | —O—(CF₂)₂—O— | |
| 617 | 3-Cl-5-CH₃—C₆H₃ | —O—(CF₂)₂—O— | |
| 618 | 3-Cl-5-CF₃—C₆H₃ | —O—(CF₂)₂—O— | |
| 619 | 3-Cl-5-OCH₃—C₆H₃ | —O—(CF₂)₂—O— | |
| 620 | 3,5-(CH₃)₂—C₆H₃ | —O—(CF₂)₂—O— | |
| 621 | 3-CH₃-5-CF₃—C₆H₃ | —O—(CF₂)₂—O— | |
| 622 | 3-CH₃-5-OCH₃—C₆H₃ | —O—(CF₂)₂—O— | |
| 623 | 3,5-(CF₃)₂—C₆H₃ | —O—(CF₂)₂—O— | |
| 624 | 3-CF₃-5-OCH₃—C₆H₃ | —O—(CF₂)₂—O— | |
| 625 | 3,5-(OCH₃)₂—C₆H₃ | —O—(CF₂)₂—O— | |
| 626 | 2,4-F₂—C₆H₃ | —O—(CF₂)₂—O— | |
| 627 | 2-F-4-Cl—C₆H₃ | —O—(CF₂)₂—O— | |
| 628 | 2-F-4-CH₃—C₆H₃ | —O—(CF₂)₂—O— | |
| 629 | 2-F-4-CF₃—C₆H₃ | —O—(CF₂)₂—O— | |
| 630 | 2-F-4-OCH₃—C₆H₃ | —O—(CF₂)₂—O— | |
| 631 | 2-Cl-4-F—C₆H₃ | —O—(CF₂)₂—O— | |
| 632 | 2,4-Cl₂—C₆H₃ | —O—(CF₂)₂—O— | |
| 633 | 2-Cl-4-CH₃—C₆H₃ | —O—(CF₂)₂—O— | |
| 634 | 2-Cl-4-CF₃—C₆H₃ | —O—(CF₂)₂—O— | |
| 635 | 2-Cl-4-OCH₃—C₆H₃ | —O—(CF₂)₂—O— | |
| 636 | 2-CH₃-4-F—C₆H₃ | —O—(CF₂)₂—O— | |
| 637 | 2-CH₃-4-Cl—C₆H₃ | —O—(CF₂)₂—O— | |
| 638 | 2,4-(CH₃)₂—C₆H₃ | —O—(CF₂)₂—O— | |
| 639 | 2-CH₃-4-CF₃—C₆H₃ | —O—(CF₂)₂—O— | |
| 640 | 2-CH₃-4-OCH₃—C₆H₃ | —O—(CF₂)₂—O— | |
| 641 | 2-CF₃-4-F—C₆H₃ | —O—(CF₂)₂—O— | |
| 642 | 2-CF₃-4-Cl—C₆H₃ | —O—(CF₂)₂—O— | |
| 643 | 2-CF₃-4-CH₃—C₆H₃ | —O—(CF₂)₂—O— | |
| 644 | 2,4-(CF₃)₂—C₆H₃ | —O—(CF₂)₂—O— | |
| 645 | 2-CF₃-4-OCH₃—C₆H₃ | —O—(CF₂)₂—O— | |
| 646 | 2-OCH₃-4-F—C₆H₃ | —O—(CF₂)₂—O— | |
| 647 | 2-OCH₃-4-Cl—C₆H₃ | —O—(CF₂)₂—O— | |
| 648 | 2-OCH₃-4-CH₃—C₆H₃ | —O—(CF₂)₂—O— | |
| 649 | 2-OCH₃-4-CF₃—C₆H₃ | —O—(CF₂)₂—O— | |
| 650 | 2,4-(OCH₃)₂—C₆H₃ | —O—(CF₂)₂—O— | |
| 651 | F | —CH₂—C(CH₃)₂—O— | |
| 652 | Cl | —CH₂—C(CH₃)₂—O— | |
| 653 | CH₃ | —CH₂—C(CH₃)₂—O— | |
| 654 | C₂H₅ | —CH₂—C(CH₃)₂—O— | |
| 655 | (CH₂)₂CH₃ | —CH₂—C(CH₃)₂—O— | |
| 656 | CH(CH₃)₂ | —CH₂—C(CH₃)₂—O— | |
| 657 | (CH₂)₃CH₃ | —CH₂—C(CH₃)₂—O— | |
| 658 | CH₂CH(CH₃)₂ | —CH₂—C(CH₃)₂—O— | |
| 659 | C(CH₃)₃ | —CH₂—C(CH₃)₂—O— | |
| 660 | C₆H₅ | —CH₂—C(CH₃)₂—O— | |
| 661 | 2-F—C₆H₄ | —CH₂—C(CH₃)₂—O— | |
| 662 | 2-Cl—C₆H₄ | —CH₂—C(CH₃)₂—O— | |
| 663 | 2-CH₃—C₆H₄ | —CH₂—C(CH₃)₂—O— | |
| 664 | 2-CF₃—C₆H₄ | —CH₂—C(CH₃)₂—O— | |
| 665 | 2-OCH₃—C₆H₄ | —CH₂—C(CH₃)₂—O— | |
| 666 | 3-F—C₆H₄ | —CH₂—C(CH₃)₂—O— | |
| 667 | 3-Cl—C₆H₄ | —CH₂—C(CH₃)₂—O— | |
| 668 | 3-CH₃—C₆H₄ | —CH₂—C(CH₃)₂—O— | |
| 669 | 3-CF₃—C₆H₄ | —CH₂—C(CH₃)₂—O— | |
| 670 | 3-OCH₃—C₆H₄ | —CH₂—C(CH₃)₂—O— | |
| 671 | 4-F—C₆H₄ | —CH₂—C(CH₃)₂—O— | |
| 672 | 4-Cl—C₆H₄ | —CH₂—C(CH₃)₂—O— | |
| 673 | 4-CH₃—C₆H₄ | —CH₂—C(CH₃)₂—O— | |
| 674 | 4-CF₃—C₆H₄ | —CH₂—C(CH₃)₂—O— | |
| 675 | 4-OCH₃—C₆H₄ | —CH₂—C(CH₃)₂—O— | |
| 676 | 3,5-F₂—C₆H₃ | —CH₂—C(CH₃)₂—O— | |
| 677 | 3-F-5-Cl—C₆H₃ | —CH₂—C(CH₃)₂—O— | |
| 678 | 3-F-5-CH₃—C₆H₃ | —CH₂—C(CH₃)₂—O— | |
| 679 | 3-F-5-CF₃—C₆H₃ | —CH₂—C(CH₃)₂—O— | |
| 680 | 3-F-5-OCH₃—C₆H₃ | —CH₂—C(CH₃)₂—O— | |
| 681 | 3,5-Cl₂—C₆H₃ | —CH₂—C(CH₃)₂—O— | |
| 682 | 3-Cl-5-CH₃—C₆H₃ | —CH₂—C(CH₃)₂—O— | |
| 683 | 3-Cl-5-CF₃—C₆H₃ | —CH₂—C(CH₃)₂—O— | |
| 684 | 3-Cl-5-OCH₃—C₆H₃ | —CH₂—C(CH₃)₂—O— | |
| 685 | 3,5-(CH₃)₂—C₆H₃ | —CH₂—C(CH₃)₂—O— | |
| 686 | 3-CH₃-5-CF₃—C₆H₃ | —CH₂—C(CH₃)₂—O— | |
| 687 | 3-CH₃-5-OCH₃—C₆H₃ | —CH₂—C(CH₃)₂—O— | |
| 688 | 3,5-(CF₃)₂—C₆H₃ | —CH₂—C(CH₃)₂—O— | |
| 689 | 3-CF₃-5-OCH₃—C₆H₃ | —CH₂—C(CH₃)₂—O— | |
| 690 | 3,5-(OCH₃)₂—C₆H₃ | —CH₂—C(CH₃)₂—O— | |
| 691 | 2,4-F₂—C₆H₃ | —CH₂—C(CH₃)₂—O— | |
| 692 | 2-F-4-Cl—C₆H₃ | —CH₂—C(CH₃)₂—O— | |
| 693 | 2-F-4-CH₃—C₆H₃ | —CH₂—C(CH₃)₂—O— | |
| 694 | 2-F-4-CF₃—C₆H₃ | —CH₂—C(CH₃)₂—O— | |
| 695 | 2-F-4-OCH₃—C₆H₃ | —CH₂—C(CH₃)₂—O— | |
| 696 | 2-Cl-4-F—C₆H₃ | —CH₂—C(CH₃)₂—O— | |
| 697 | 2,4-Cl₂—C₆H₃ | —CH₂—C(CH₃)₂—O— | |
| 698 | 2-Cl-4-CH₃—C₆H₃ | —CH₂—C(CH₃)₂—O— | |
| 699 | 2-Cl-4-CF₃—C₆H₃ | —CH₂—C(CH₃)₂—O— | |
| 700 | 2-Cl-4-OCH₃—C₆H₃ | —CH₂—C(CH₃)₂—O— | |
| 701 | 2-CH₃-4-F—C₆H₃ | —CH₂—C(CH₃)₂—O— | |
| 702 | 2-CH₃-4-Cl—C₆H₃ | —CH₂—C(CH₃)₂—O— | |
| 703 | 2,4-(CH₃)₂—C₆H₃ | —CH₂—C(CH₃)₂—O— | |
| 704 | 2-CH₃-4-CF₃—C₆H₃ | —CH₂—C(CH₃)₂—O— | |
| 705 | 2-CH₃-4-OCH₃—C₆H₃ | —CH₂—C(CH₃)₂—O— | |
| 706 | 2-CF₃-4-F—C₆H₃ | —CH₂—C(CH₃)₂—O— | |
| 707 | 2-CF₃-4-Cl—C₆H₃ | —CH₂—C(CH₃)₂—O— | |
| 708 | 2-CF₃-4-CH₃—C₆H₃ | —CH₂—C(CH₃)₂—O— | |
| 709 | 2,4-(CF₃)₂—C₆H₃ | —CH₂—C(CH₃)₂—O— | |
| 710 | 2-CF₃-4-OCH₃—C₆H₃ | —CH₂—C(CH₃)₂—O— | |
| 711 | 2-OCH₃-4-F—C₆H₃ | —CH₂—C(CH₃)₂—O— | |
| 712 | 2-OCH₃-4-Cl—C₆H₃ | —CH₂—C(CH₃)₂—O— | |
| 713 | 2-OCH₃-4-CH₃—C₆H₃ | —CH₂—C(CH₃)₂—O— | |
| 714 | 2-OCH₃-4-CF₃—C₆H₃ | —CH₂—C(CH₃)₂—O— | |
| 715 | 2,4-(OCH₃)₂—C₆H₃ | —CH₂—C(CH₃)₂—O— | |
| 716 | F | —CH₂—CHCH₃—O— | |
| 717 | Cl | —CH₂—CHCH₃—O— | |
| 718 | CH₃ | —CH₂—CHCH₃—O— | |
| 719 | C₂H₅ | —CH₂—CHCH₃—O— | |
| 720 | (CH₂)₂CH₃ | —CH₂—CHCH₃—O— | |
| 721 | CH(CH₃)₂ | —CH₂—CHCH₃—O— | |
| 722 | (CH₂)₃CH₃ | —CH₂—CHCH₃—O— | |
| 723 | CH₂CH(CH₃)₂ | —CH₂—CHCH₃—O— | |
| 724 | C(CH₃)₃ | —CH₂—CHCH₃—O— | |
| 725 | C₆H₅ | —CH₂—CHCH₃—O— | |
| 726 | 2-F—C₆H₄ | —CH₂—CHCH₃—O— | |
| 727 | 2-Cl—C₆H₄ | —CH₂—CHCH₃—O— | |
| 728 | 2-CH₃—C₆H₄ | —CH₂—CHCH₃—O— | |
| 729 | 2-CF₃—C₆H₄ | —CH₂—CHCH₃—O— | |
| 730 | 2-OCH₃—C₆H₄ | —CH₂—CHCH₃—O— | |
| 731 | 3-F—C₆H₄ | —CH₂—CHCH₃—O— | |
| 732 | 3-Cl—C₆H₄ | —CH₂—CHCH₃—O— | |
| 733 | 3-CH₃—C₆H₄ | —CH₂—CHCH₃—O— | |
| 734 | 3-CF₃—C₆H₄ | —CH₂—CHCH₃—O— | |
| 735 | 3-OCH₃—C₆H₄ | —CH₂—CHCH₃—O— | |
| 736 | 4-F—C₆H₄ | —CH₂—CHCH₃—O— | |
| 737 | 4-Cl—C₆H₄ | —CH₂—CHCH₃—O— | |
| 738 | 4-CH₃—C₆H₄ | —CH₂—CHCH₃—O— | |
| 739 | 4-CF₃—C₆H₄ | —CH₂—CHCH₃—O— | |
| 740 | 4-OCH₃—C₆H₄ | —CH₂—CHCH₃—O— | |
| 741 | 3,5-F₂—C₆H₃ | —CH₂—CHCH₃—O— | |
| 742 | 3-F-5-Cl—C₆H₃ | —CH₂—CHCH₃—O— | |
| 743 | 3-F-5-CH₃—C₆H₃ | —CH₂—CHCH₃—O— | |
| 744 | 3-F-5-CF₃—C₆H₃ | —CH₂—CHCH₃—O— | |
| 745 | 3-F-5-OCH₃—C₆H₃ | —CH₂—CHCH₃—O— | |
| 746 | 3,5-Cl₂—C₆H₃ | —CH₂—CHCH₃—O— | |
| 747 | 3-Cl-5-CH₃—C₆H₃ | —CH₂—CHCH₃—O— | |
| 748 | 3-Cl-5-CF₃—C₆H₃ | —CH₂—CHCH₃—O— | |
| 749 | 3-Cl-5-OCH₃—C₆H₃ | —CH₂—CHCH₃—O— | |
| 750 | 3,5-(CH₃)₂—C₆H₃ | —CH₂—CHCH₃—O— | |
| 751 | 3-CH₃-5-CF₃—C₆H₃ | —CH₂—CHCH₃—O— | |
| 752 | 3-CH₃-5-OCH₃—C₆H₃ | —CH₂—CHCH₃—O— | |
| 753 | 3,5-(CF₃)₂—C₆H₃ | —CH₂—CHCH₃—O— | |
| 754 | 3-CF₃-5-OCH₃—C₆H₃ | —CH₂—CHCH₃—O— | |
| 755 | 3,5-(OCH₃)₂—C₆H₃ | —CH₂—CHCH₃—O— | |
| 756 | 2,4-F₂—C₆H₃ | —CH₂—CHCH₃—O— | |
| 757 | 2-F-4-Cl—C₆H₃ | —CH₂—CHCH₃—O— | |
| 758 | 2-F-4-CH₃—C₆H₃ | —CH₂—CHCH₃—O— | |
| 759 | 2-F-4-CF₃—C₆H₃ | —CH₂—CHCH₃—O— | |
| 760 | 2-F-4-OCH₃—C₆H₃ | —CH₂—CHCH₃—O— | |
| 761 | 2-Cl-4-F—C₆H₃ | —CH₂—CHCH₃—O— | |
| 762 | 2,4-Cl₂—C₆H₃ | —CH₂—CHCH₃—O— | |
| 763 | 2-Cl-4-CH₃—C₆H₃ | —CH₂—CHCH₃—O— | |
| 764 | 2-Cl-4-CF₃—C₆H₃ | —CH₂—CHCH₃—O— | |
| 765 | 2-Cl-4-OCH₃—C₆H₃ | —CH₂—CHCH₃—O— | |
| 766 | 2-CH₃-4-F—C₆H₃ | —CH₂—CHCH₃—O— | |
| 767 | 2-CH₃-4-Cl—C₆H₃ | —CH₂—CHCH₃—O— | |
| 768 | 2,4-(CH₃)₂—C₆H₃ | —CH₂—CHCH₃—O— | |
| 769 | 2-CH₃-4-CF₃—C₆H₃ | —CH₂—CHCH₃—O— | |
| 770 | 2-CH₃-4-OCH₃—C₆H₃ | —CH₂—CHCH₃—O— | |
| 771 | 2-CF₃-4-F—C₆H₃ | —CH₂—CHCH₃—O— | |
| 772 | 2-CF₃-4-Cl—C₆H₃ | —CH₂—CHCH₃—O— | |
| 773 | 2-CF₃-4-CH₃—C₆H₃ | —CH₂—CHCH₃—O— | |
| 774 | 2,4-(CF₃)₂—C₆H₃ | —CH₂—CHCH₃—O— | |
| 775 | 2-CF₃-4-OCH₃—C₆H₃ | —CH₂—CHCH₃—O— | |
| 776 | 2-OCH₃-4-F—C₆H₃ | —CH₂—CHCH₃—O— | |
| 777 | 2-OCH₃-4-Cl—C₆H₃ | —CH₂—CHCH₃—O— | |
| 778 | 2-OCH₃-4-CH₃—C₆H₃ | —CH₂—CHCH₃—O— | |
| 779 | 2-OCH₃-4-CF₃—C₆H₃ | —CH₂—CHCH₃—O— | |
| 780 | 2,4-(OCH₃)₂—C₆H₃ | —CH₂—CHCH₃—O— | |
| 781 | F | —CHCH₃—CH₂—O— | |
| 782 | Cl | —CHCH₃—CH₂—O— | |
| 783 | CH₃ | —CHCH₃—CH₂—O— | |
| 784 | C₂H₅ | —CHCH₃—CH₂—O— | |
| 785 | (CH₂)₂CH₃ | —CHCH₃—CH₂—O— | |
| 786 | CH(CH₃)₂ | —CHCH₃—CH₂—O— | |
| 787 | (CH₂)₃CH₃ | —CHCH₃—CH₂—O— | |
| 788 | CH₂CH(CH₃)₂ | —CHCH₃—CH₂—O— | |
| 789 | C(CH₃)₃ | —CHCH₃—CH₂—O— | |
| 790 | C₆H₅ | —CHCH₃—CH₂—O— | |
| 791 | 2-F—C₆H₄ | —CHCH₃—CH₂—O— | |
| 792 | 2-Cl—C₆H₄ | —CHCH₃—CH₂—O— | |
| 793 | 2-CH₃—C₆H₄ | —CHCH₃—CH₂—O— | |
| 794 | 2-CF₃—C₆H₄ | —CHCH₃—CH₂—O— | |
| 795 | 2-OCH₃—C₆H₄ | —CHCH₃—CH₂—O— | |
| 796 | 3-F—C₆H₄ | —CHCH₃—CH₂—O— | |
| 797 | 3-Cl—C₆H₄ | —CHCH₃—CH₂—O— | |
| 798 | 3-CH₃—C₆H₄ | —CHCH₃—CH₂—O— | |
| 799 | 3-CF₃—C₆H₄ | —CHCH₃—CH₂—O— | |
| 800 | 3-OCH₃—C₆H₄ | —CHCH₃—CH₂—O— | |
| 801 | 4-F—C₆H₄ | —CHCH₃—CH₂—O— | |
| 802 | 4-Cl—C₆H₄ | —CHCH₃—CH₂—O— | |
| 803 | 4-CH₃—C₆H₄ | —CHCH₃—CH₂—O— | |
| 804 | 4-CF₃—C₆H₄ | —CHCH₃—CH₂—O— | |
| 805 | 4-OCH₃—C₆H₄ | —CHCH₃—CH₂—O— | |
| 806 | 3,5-F₂—C₆H₃ | —CHCH₃—CH₂—O— | |
| 807 | 3-F-5-Cl—C₆H₃ | —CHCH₃—CH₂—O— | |
| 808 | 3-F-5-CH₃—C₆H₃ | —CHCH₃—CH₂—O— | |
| 809 | 3-F-5-CF₃—C₆H₃ | —CHCH₃—CH₂—O— | |
| 810 | 3-F-5-OCH₃—C₆H₃ | —CHCH₃—CH₂—O— | |
| 811 | 3,5-Cl₂—C₆H₃ | —CHCH₃—CH₂—O— | |
| 812 | 3-Cl-5-CH₃—C₆H₃ | —CHCH₃—CH₂—O— | |
| 813 | 3-Cl-5-CF₃—C₆H₃ | —CHCH₃—CH₂—O— | |
| 814 | 3-Cl-5-OCH₃—C₆H₃ | —CHCH₃—CH₂—O— | |
| 815 | 3,5-(CH₃)₂—C₆H₃ | —CHCH₃—CH₂—O— | |
| 816 | 3-CH₃-5-CF₃—C₆H₃ | —CHCH₃—CH₂—O— | |
| 817 | 3-CH₃-5-OCH₃—C₆H₃ | —CHCH₃—CH₂—O— | |
| 818 | 3,5-(CF₃)₂—C₆H₃ | —CHCH₃—CH₂—O— | |
| 819 | 3-CF₃-5-OCH₃—C₆H₃ | —CHCH₃—CH₂—O— | |
| 820 | 3,5-(OCH₃)₂—C₆H₃ | —CHCH₃—CH₂—O— | |
| 821 | 2,4-F₂—C₆H₃ | —CHCH₃—CH₂—O— | |
| 822 | 2-F-4-Cl—C₆H₃ | —CHCH₃—CH₂—O— | |
| 823 | 2-F-4-CH₃—C₆H₃ | —CHCH₃—CH₂—O— | |
| 824 | 2-F-4-CF₃—C₆H₃ | —CHCH₃—CH₂—O— | |
| 825 | 2-F-4-OCH₃—C₆H₃ | —CHCH₃—CH₂—O— | |
| 826 | 2-Cl-4-F—C₆H₃ | —CHCH₃—CH₂—O— | |
| 827 | 2,4-Cl₂—C₆H₃ | —CHCH₃—CH₂—O— | |
| 828 | 2-Cl-4-CH₃—C₆H₃ | —CHCH₃—CH₂—O— | |
| 829 | 2-Cl-4-CF₃—C₆H₃ | —CHCH₃—CH₂—O— | |
| 830 | 2-Cl-4-OCH₃—C₆H₃ | —CHCH₃—CH₂—O— | |
| 831 | 2-CH₃-4-F—C₆H₃ | —CHCH₃—CH₂—O— | |
| 832 | 2-CH₃-4-Cl—C₆H₃ | —CHCH₃—CH₂—O— | |
| 833 | 2,4-(CH₃)₂—C₆H₃ | —CHCH₃—CH₂—O— | |
| 834 | 2-CH₃-4-CF₃—C₆H₃ | —CHCH₃—CH₂—O— | |
| 835 | 2-CH₃-4-OCH₃—C₆H₃ | —CHCH₃—CH₂—O— | |
| 836 | 2-CF₃-4-F—C₆H₃ | —CHCH₃—CH₂—O— | |
| 837 | 2-CF₃-4-Cl—C₆H₃ | —CHCH₃—CH₂—O— | |
| 838 | 2-CF₃-4-CH₃—C₆H₃ | —CHCH₃—CH₂—O— | |
| 839 | 2,4-(CF₃)₂—C₆H₃ | —CHCH₃—CH₂—O— | |
| 840 | 2-CF₃-4-OCH₃—C₆H₃ | —CHCH₃—CH₂—O— | |
| 841 | 2-OCH₃-4-F—C₆H₃ | —CHCH₃—CH₂—O— | |
| 842 | 2-OCH₃-4-Cl—C₆H₃ | —CHCH₃—CH₂—O— | |
| 843 | 2-OCH₃-4-CH₃—C₆H₃ | —CHCH₃—CH₂—O— | |
| 844 | 2-OCH₃-4-CF₃—C₆H₃ | —CHCH₃—CH₂—O— | |
| 845 | 2,4-(OCH₃)₂—C₆H₃ | —CHCH₃—CH₂—O— | |
| 846 | F | —O—CHCH₃—CHCH₃— | |
| 847 | Cl | —O—CHCH₃—CHCH₃— | |
| 848 | CH₃ | —O—CHCH₃—CHCH₃— | |
| 849 | C₂H₅ | —O—CHCH₃—CHCH₃— | |
| 850 | (CH₂)₂CH₃ | —O—CHCH₃—CHCH₃— | |
| 851 | CH(CH₃)₂ | —O—CHCH₃—CHCH₃— | |
| 852 | (CH₂)₃CH₃ | —O—CHCH₃—CHCH₃— | |
| 853 | CH₂CH(CH₃)₂ | —O—CHCH₃—CHCH₃— | |
| 854 | C(CH₃)₃ | —O—CHCH₃—CHCH₃— | |
| 855 | C₆H₅ | —O—CHCH₃—CHCH₃— | |
| 856 | 2-F—C₆H₄ | —O—CHCH₃—CHCH₃— | |
| 857 | 2-Cl—C₆H₄ | —O—CHCH₃—CHCH₃— | |
| 858 | 2-CH₃—C₆H₄ | —O—CHCH₃—CHCH₃— | |
| 859 | 2-CF₃—C₆H₄ | —O—CHCH₃—CHCH₃— | |
| 860 | 2-OCH₃—C₆H₄ | —O—CHCH₃—CHCH₃— | |
| 861 | 3-F—C₆H₄ | —O—CHCH₃—CHCH₃— | |
| 862 | 3-Cl—C₆H₄ | —O—CHCH₃—CHCH₃— | |
| 863 | 3-CH₃—C₆H₄ | —O—CHCH₃—CHCH₃— | |
| 864 | 3-CF₃—C₆H₄ | —O—CHCH₃—CHCH₃— | |
| 865 | 3-OCH₃—C₆H₄ | —O—CHCH₃—CHCH₃— | |
| 866 | 4-F—C₆H₄ | —O—CHCH₃—CHCH₃— | |
| 867 | 4-Cl—C₆H₄ | —O—CHCH₃—CHCH₃— | |
| 868 | 4-CH₃—C₆H₄ | —O—CHCH₃—CHCH₃— | |
| 869 | 4-CF₃—C₆H₄ | —O—CHCH₃—CHCH₃— | |
| 870 | 4-OCH₃—C₆H₄ | —O—CHCH₃—CHCH₃— | |
| 871 | 3,5-F₂—C₆H₃ | —O—CHCH₃—CHCH₃— | |
| 872 | 3-F-5-Cl—C₆H₃ | —O—CHCH₃—CHCH₃— | |
| 873 | 3-F-5-CH₃—C₆H₃ | —O—CHCH₃—CHCH₃— | |
| 874 | 3-F-5-CF₃—C₆H₃ | —O—CHCH₃—CHCH₃— | |
| 875 | 3-F-5-OCH₃—C₆H₃ | —O—CHCH₃—CHCH₃— | |
| 876 | 3,5-Cl₂—C₆H₃ | —O—CHCH₃—CHCH₃— | |
| 877 | 3-Cl-5-CH₃—C₆H₃ | —O—CHCH₃—CHCH₃— | |
| 878 | 3-Cl-5-CF₃—C₆H₃ | —O—CHCH₃—CHCH₃— | |
| 879 | 3-Cl-5-OCH₃—C₆H₃ | —O—CHCH₃—CHCH₃— | |
| 880 | 3,5-(CH₃)₂—C₆H₃ | —O—CHCH₃—CHCH₃— | |
| 881 | 3-CH₃-5-CF₃—C₆H₃ | —O—CHCH₃—CHCH₃— | |
| 882 | 3-CH₃-5-OCH₃—C₆H₃ | —O—CHCH₃—CHCH₃— | |
| 883 | 3,5-(CF₃)₂—C₆H₃ | —O—CHCH₃—CHCH₃— | |
| 884 | 3-CF₃-5-OCH₃—C₆H₃ | —O—CHCH₃—CHCH₃— | |
| 885 | 3,5-(OCH₃)₂—C₆H₃ | —O—CHCH₃—CHCH₃— | |
| 886 | 2,4-F₂—C₆H₃ | —O—CHCH₃—CHCH₃— | |
| 887 | 2-F-4-Cl—C₆H₃ | —O—CHCH₃—CHCH₃— | |
| 888 | 2-F-4-CH₃—C₆H₃ | —O—CHCH₃—CHCH₃— | |
| 889 | 2-F-4-CF₃—C₆H₃ | —O—CHCH₃—CHCH₃— | |
| 890 | 2-F-4-OCH₃—C₆H₃ | —O—CHCH₃—CHCH₃— | |
| 891 | 2-Cl-4-F—C₆H₃ | —O—CHCH₃—CHCH₃— | |
| 892 | 2,4-Cl₂—C₆H₃ | —O—CHCH₃—CHCH₃— | |
| 893 | 2-Cl-4-CH₃—C₆H₃ | —O—CHCH₃—CHCH₃— | |
| 894 | 2-Cl-4-CF₃—C₆H₃ | —O—CHCH₃—CHCH₃— | |
| 895 | 2-Cl-4-OCH₃—C₆H₃ | —O—CHCH₃—CHCH₃— | |
| 896 | 2-CH₃-4-F—C₆H₃ | —O—CHCH₃—CHCH₃— | |
| 897 | 2-CH₃-4-Cl—C₆H₃ | —O—CHCH₃—CHCH₃— | |
| 898 | 2,4-(CH₃)₂—C₆H₃ | —O—CHCH₃—CHCH₃— | |
| 899 | 2-CH₃-4-CF₃—C₆H₃ | —O—CHCH₃—CHCH₃— | |
| 900 | 2-CH₃-4-OCH₃—C₆H₃ | —O—CHCH₃—CHCH₃— | |
| 901 | 2-CF₃-4-F—C₆H₃ | —O—CHCH₃—CHCH₃— | |
| 902 | 2-CF₃-4-Cl—C₆H₃ | —O—CHCH₃—CHCH₃— | |
| 903 | 2-CF₃-4-CH₃—C₆H₃ | —O—CHCH₃—CHCH₃— | |
| 904 | 2,4-(CF₃)₂—C₆H₃ | —O—CHCH₃—CHCH₃— | |
| 905 | 2-CF₃-4-OCH₃—C₆H₃ | —O—CHCH₃—CHCH₃— | |
| 906 | 2-OCH₃-4-F—C₆H₃ | —O—CHCH₃—CHCH₃— | |
| 907 | 2-OCH₃-4-Cl—C₆H₃ | —O—CHCH₃—CHCH₃— | |
| 908 | 2-OCH₃-4-CH₃—C₆H₃ | —O—CHCH₃—CHCH₃— | |
| 909 | 2-OCH₃-4-CF₃—C₆H₃ | —O—CHCH₃—CHCH₃— | |
| 910 | 2,4-(OCH₃)₂—C₆H₃ | —O—CHCH₃—CHCH₃— | |
| 911 | F | —O—CH₂—C(CH₃)₂— | |
| 912 | Cl | —O—CH₂—C(CH₃)₂— | |
| 913 | CH₃ | —O—CH₂—C(CH₃)₂— | |
| 914 | C₂H₅ | —O—CH₂—C(CH₃)₂— | |
| 915 | (CH₂)₂CH₃ | —O—CH₂—C(CH₃)₂— | |
| 916 | CH(CH₃)₂ | —O—CH₂—C(CH₃)₂— | |
| 917 | (CH₂)₃CH₃ | —O—CH₂—C(CH₃)₂— | |
| 918 | CH₂CH(CH₃)₂ | —O—CH₂—C(CH₃)₂— | |
| 919 | C(CH₃)₃ | —O—CH₂—C(CH₃)₂— | |
| 920 | C₆H₅ | —O—CH₂—C(CH₃)₂— | |
| 921 | 2-F—C₆H₄ | —O—CH₂—C(CH₃)₂— | |
| 922 | 2-Cl—C₆H₄ | —O—CH₂—C(CH₃)₂— | |
| 923 | 2-CH₃—C₆H₄ | —O—CH₂—C(CH₃)₂— | |
| 924 | 2-CF₃—C₆H₄ | —O—CH₂—C(CH₃)₂— | |
| 925 | 2-OCH₃—C₆H₄ | —O—CH₂—C(CH₃)₂— | |
| 926 | 3-F—C₆H₄ | —O—CH₂—C(CH₃)₂— | |
| 927 | 3-Cl—C₆H₄ | —O—CH₂—C(CH₃)₂— | |
| 928 | 3-CH₃—C₆H₄ | —O—CH₂—C(CH₃)₂— | |
| 929 | 3-CF₃—C₆H₄ | —O—CH₂—C(CH₃)₂— | |
| 930 | 3-OCH₃—C₆H₄ | —O—CH₂—C(CH₃)₂— | |
| 931 | 4-F—C₆H₄ | —O—CH₂—C(CH₃)₂— | |
| 932 | 4-Cl—C₆H₄ | —O—CH₂—C(CH₃)₂— | |
| 933 | 4-CH₃—C₆H₄ | —O—CH₂—C(CH₃)₂— | |
| 934 | 4-CF₃—C₆H₄ | —O—CH₂—C(CH₃)₂— | |
| 935 | 4-OCH₃—C₆H₄ | —O—CH₂—C(CH₃)₂— | |
| 936 | 3,5-F₂—C₆H₃ | —O—CH₂—C(CH₃)₂— | |
| 937 | 3-F-5-Cl—C₆H₃ | —O—CH₂—C(CH₃)₂— | |
| 938 | 3-F-5-CH₃—C₆H₃ | —O—CH₂—C(CH₃)₂— | |
| 939 | 3-F-5-CF₃—C₆H₃ | —O—CH₂—C(CH₃)₂— | |
| 940 | 3-F-5-OCH₃—C₆H₃ | —O—CH₂—C(CH₃)₂— | |
| 941 | 3,5-Cl₂—C₆H₃ | —O—CH₂—C(CH₃)₂— | |
| 942 | 3-Cl-5-CH₃—C₆H₃ | —O—CH₂—C(CH₃)₂— | |
| 943 | 3-Cl-5-CF₃—C₆H₃ | —O—CH₂—C(CH₃)₂— | |
| 944 | 3-Cl-5-OCH₃—C₆H₃ | —O—CH₂—C(CH₃)₂— | |
| 945 | 3,5-(CH₃)₂—C₆H₃ | —O—CH₂—C(CH₃)₂— | |
| 946 | 3-CH₃-5-CF₃—C₆H₃ | —O—CH₂—C(CH₃)₂— | |
| 947 | 3-CH₃-5-OCH₃—C₆H₃ | —O—CH₂—C(CH₃)₂— | |
| 948 | 3,5-(CF₃)₂—C₆H₃ | —O—CH₂—C(CH₃)₂— | |
| 949 | 3-CF₃-5-OCH₃—C₆H₃ | —O—CH₂—C(CH₃)₂— | |
| 950 | 3,5-(OCH₃)₂—C₆H₃ | —O—CH₂—C(CH₃)₂— | |
| 951 | 2,4-F₂—C₆H₃ | —O—CH₂—C(CH₃)₂— | |
| 952 | 2-F-4-Cl—C₆H₃ | —O—CH₂—C(CH₃)₂— | |
| 953 | 2-F-4-CH₃—C₆H₃ | —O—CH₂—C(CH₃)₂— | |
| 954 | 2-F-4-CF₃—C₆H₃ | —O—CH₂—C(CH₃)₂— | |
| 955 | 2-F-4-OCH₃—C₆H₃ | —O—CH₂—C(CH₃)₂— | |
| 956 | 2-Cl-4-F—C₆H₃ | —O—CH₂—C(CH₃)₂— | |
| 957 | 2,4-Cl₂—C₆H₃ | —O—CH₂—C(CH₃)₂— | |
| 958 | 2-Cl-4-CH₃—C₆H₃ | —O—CH₂—C(CH₃)₂— | |
| 959 | 2-Cl-4-CF₃—C₆H₃ | —O—CH₂—C(CH₃)₂— | |
| 960 | 2-Cl-4-OCH₃—C₆H₃ | —O—CH₂—C(CH₃)₂— | |
| 961 | 2-CH₃-4-F—C₆H₃ | —O—CH₂—C(CH₃)₂— | |
| 962 | 2-CH₃-4-Cl—C₆H₃ | —O—CH₂—C(CH₃)₂— | |
| 963 | 2,4-(CH₃)₂—C₆H₃ | —O—CH₂—C(CH₃)₂— | |
| 964 | 2-CH₃-4-CF₃—C₆H₃ | —O—CH₂—C(CH₃)₂— | |
| 965 | 2-CH₃-4-OCH₃—C₆H₃ | —O—CH₂—C(CH₃)₂— | |
| 966 | 2-CF₃-4-F—C₆H₃ | —O—CH₂—C(CH₃)₂— | |
| 967 | 2-CF₃-4-Cl—C₆H₃ | —O—CH₂—C(CH₃)₂— | |
| 968 | 2-CF₃-4-CH₃—C₆H₃ | —O—CH₂—C(CH₃)₂— | |
| 969 | 2,4-(CF₃)₂—C₆H₃ | —O—CH₂—C(CH₃)₂— | |
| 970 | 2-CF₃-4-OCH₃—C₆H₃ | —O—CH₂—C(CH₃)₂— | |
| 971 | 2-OCH₃-4-F—C₆H₃ | —O—CH₂—C(CH₃)₂— | |
| 972 | 2-OCH₃-4-Cl—C₆H₃ | —O—CH₂—C(CH₃)₂— | |
| 973 | 2-OCH₃-4-CH₃—C₆H₃ | —O—CH₂—C(CH₃)₂— | |
| 974 | 2-OCH₃-4-CF₃—C₆H₃ | —O—CH₂—C(CH₃)₂— | |
| 975 | 2,4-(OCH₃)₂—C₆H₃ | —O—CH₂—C(CH₃)₂— | |
| 976 | F | —O—CH₂—CHCH₃— | |
| 977 | Cl | —O—CH₂—CHCH₃— | |
| 978 | CH₃ | —O—CH₂—CHCH₃— | |
| 979 | C₂H₅ | —O—CH₂—CHCH₃— | |
| 980 | (CH₂)₂CH₃ | —O—CH₂—CHCH₃— | |
| 981 | CH(CH₃)₂ | —O—CH₂—CHCH₃— | |
| 982 | (CH₂)₃CH₃ | —O—CH₂—CHCH₃— | |
| 983 | CH₂CH(CH₃)₂ | —O—CH₂—CHCH₃— | |
| 984 | C(CH₃)₃ | —O—CH₂—CHCH₃— | |
| 985 | C₆H₅ | —O—CH₂—CHCH₃— | |
| 986 | 2-F—C₆H₄ | —O—CH₂—CHCH₃— | |
| 987 | 2-Cl—C₆H₄ | —O—CH₂—CHCH₃— | |
| 988 | 2-CH₃—C₆H₄ | —O—CH₂—CHCH₃— | |
| 989 | 2-CF₃—C₆H₄ | —O—CH₂—CHCH₃— | |
| 990 | 2-OCH₃—C₆H₄ | —O—CH₂—CHCH₃— | |
| 991 | 3-F—C₆H₄ | —O—CH₂—CHCH₃— | |
| 992 | 3-Cl—C₆H₄ | —O—CH₂—CHCH₃— | |
| 993 | 3-CH₃—C₆H₄ | —O—CH₂—CHCH₃— | |
| 994 | 3-CF₃—C₆H₄ | —O—CH₂—CHCH₃— | |
| 995 | 3-OCH₃—C₆H₄ | —O—CH₂—CHCH₃— | |
| 996 | 4-F—C₆H₄ | —O—CH₂—CHCH₃— | |
| 997 | 4-Cl—C₆H₄ | —O—CH₂—CHCH₃— | |
| 998 | 4-CH₃—C₆H₄ | —O—CH₂—CHCH₃— | |
| 999 | 4-CF₃—C₆H₄ | —O—CH₂—CHCH₃— | |
| 1000 | 4-OCH₃—C₆H₄ | —O—CH₂—CHCH₃— | |
| 1001 | 3,5-F₂—C₆H₃ | —O—CH₂—CHCH₃— | |
| 1002 | 3-F-5-Cl—C₆H₃ | —O—CH₂—CHCH₃— | |
| 1003 | 3-F-5-CH₃—C₆H₃ | —O—CH₂—CHCH₃— | |
| 1004 | 3-F-5-CF₃—C₆H₃ | —O—CH₂—CHCH₃— | |
| 1005 | 3-F-5-OCH₃—C₆H₃ | —O—CH₂—CHCH₃— | |
| 1006 | 3,5-Cl₂—C₆H₃ | —O—CH₂—CHCH₃— | |
| 1007 | 3-Cl-5-CH₃—C₆H₃ | —O—CH₂—CHCH₃— | |
| 1008 | 3-Cl-5-CF₃—C₆H₃ | —O—CH₂—CHCH₃— | |
| 1009 | 3-Cl-5-OCH₃—C₆H₃ | —O—CH₂—CHCH₃— | |
| 1010 | 3,5-(CH₃)₂—C₆H₃ | —O—CH₂—CHCH₃— | |
| 1011 | 3-CH₃-5-CF₃—C₆H₃ | —O—CH₂—CHCH₃— | |
| 1012 | 3-CH₃-5-OCH₃—C₆H₃ | —O—CH₂—CHCH₃— | |
| 1013 | 3,5-(CF₃)₂—C₆H₃ | —O—CH₂—CHCH₃— | |
| 1014 | 3-CF₃-5-OCH₃—C₆H₃ | —O—CH₂—CHCH₃— | |
| 1015 | 3,5-(OCH₃)₂—C₆H₃ | —O—CH₂—CHCH₃— | |
| 1016 | 2,4-F₂—C₆H₃ | —O—CH₂—CHCH₃— | |
| 1017 | 2-F-4-Cl—C₆H₃ | —O—CH₂—CHCH₃— | |
| 1018 | 2-F-4-CH₃—C₆H₃ | —O—CH₂—CHCH₃— | |
| 1019 | 2-F-4-CF₃—C₆H₃ | —O—CH₂—CHCH₃— | |
| 1020 | 2-F-4-OCH₃—C₆H₃ | —O—CH₂—CHCH₃— | |
| 1021 | 2-Cl-4-F—C₆H₃ | —O—CH₂—CHCH₃— | |
| 1022 | 2,4-Cl₂—C₆H₃ | —O—CH₂—CHCH₃— | |
| 1023 | 2-Cl-4-CH₃—C₆H₃ | —O—CH₂—CHCH₃— | |
| 1024 | 2-Cl-4-CF₃—C₆H₃ | —O—CH₂—CHCH₃— | |
| 1025 | 2-Cl-4-OCH₃—C₆H₃ | —O—CH₂—CHCH₃— | |
| 1026 | 2-CH₃-4-F—C₆H₃ | —O—CH₂—CHCH₃— | |
| 1027 | 2-CH₃-4-Cl—C₆H₃ | —O—CH₂—CHCH₃— | |
| 1028 | 2,4-(CH₃)₂—C₆H₃ | —O—CH₂—CHCH₃— | |
| 1029 | 2-CH₃-4-CF₃—C₆H₃ | —O—CH₂—CHCH₃— | |
| 1030 | 2-CH₃-4-OCH₃—C₆H₃ | —O—CH₂—CHCH₃— | |
| 1031 | 2-CF₃-4-F—C₆H₃ | —O—CH₂—CHCH₃— | |
| 1032 | 2-CF₃-4-Cl—C₆H₃ | —O—CH₂—CHCH₃— | |
| 1033 | 2-CF₃-4-CH₃—C₆H₃ | —O—CH₂—CHCH₃— | |
| 1034 | 2,4-(CF₃)₂—C₆H₃ | —O—CH₂—CHCH₃— | |
| 1035 | 2-CF₃-4-OCH₃—C₆H₃ | —O—CH₂—CHCH₃— | |
| 1036 | 2-OCH₃-4-F—C₆H₃ | —O—CH₂—CHCH₃— | |
| 1037 | 2-OCH₃-4-Cl—C₆H₃ | —O—CH₂—CHCH₃— | |
| 1038 | 2-OCH₃-4-CH₃—C₆H₃ | —O—CH₂—CHCH₃— | |
| 1039 | 2-OCH₃-4-CF₃—C₆H₃ | —O—CH₂—CHCH₃— | |
| 1040 | 2,4-(OCH₃)₂—C₆H₃ | —O—CH₂—CHCH₃— | |
| 1041 | F | —O—CHCH₃—CH₂— | |
| 1042 | Cl | —O—CHCH₃—CH₂— | |
| 1043 | CH₃ | —O—CHCH₃—CH₂— | |
| 1044 | C₂H₅ | —O—CHCH₃—CH₂— | |
| 1045 | (CH₂)₂CH₃ | —O—CHCH₃—CH₂— | |
| 1046 | CH(CH₃)₂ | —O—CHCH₃—CH₂— | |
| 1047 | (CH₂)₃CH₃ | —O—CHCH₃—CH₂— | |
| 1048 | CH₂CH(CH₃)₂ | —O—CHCH₃—CH₂— | |
| 1049 | C(CH₃)₃ | —O—CHCH₃—CH₂— | |
| 1050 | C₆H₅ | —O—CHCH₃—CH₂— | |
| 1051 | 2-F—C₆H₄ | —O—CHCH₃—CH₂— | |
| 1052 | 2-Cl—C₆H₄ | —O—CHCH₃—CH₂— | |
| 1053 | 2-CH₃—C₆H₄ | —O—CHCH₃—CH₂— | |
| 1054 | 2-CF₃—C₆H₄ | —O—CHCH₃—CH₂— | |
| 1055 | 2-OCH₃—C₆H₄ | —O—CHCH₃—CH₂— | |
| 1056 | 3-F—C₆H₄ | —O—CHCH₃—CH₂— | |
| 1057 | 3-Cl—C₆H₄ | —O—CHCH₃—CH₂— | |
| 1058 | 3-CH₃—C₆H₄ | —O—CHCH₃—CH₂— | |
| 1059 | 3-CF₃—C₆H₄ | —O—CHCH₃—CH₂— | |
| 1060 | 3-OCH₃—C₆H₄ | —O—CHCH₃—CH₂— | |
| 1061 | 4-F—C₆H₄ | —O—CHCH₃—CH₂— | |
| 1062 | 4-Cl—C₆H₄ | —O—CHCH₃—CH₂— | |
| 1063 | 4-CH₃—C₆H₄ | —O—CHCH₃—CH₂— | |
| 1064 | 4-CF₃—C₆H₄ | —O—CHCH₃—CH₂— | |
| 1065 | 4-OCH₃—C₆H₄ | —O—CHCH₃—CH₂— | |
| 1066 | 3,5-F₂—C₆H₃ | —O—CHCH₃—CH₂— | |
| 1067 | 3-F-5-Cl—C₆H₃ | —O—CHCH₃—CH₂— | |
| 1068 | 3-F-5-CH₃—C₆H₃ | —O—CHCH₃—CH₂— | |
| 1069 | 3-F-5-CF₃—C₆H₃ | —O—CHCH₃—CH₂— | |
| 1070 | 3-F-5-OCH₃—C₆H₃ | —O—CHCH₃—CH₂— | |
| 1071 | 3,5-Cl₂—C₆H₃ | —O—CHCH₃—CH₂— | |
| 1072 | 3-Cl-5-CH₃—C₆H₃ | —O—CHCH₃—CH₂— | |
| 1073 | 3-Cl-5-CF₃—C₆H₃ | —O—CHCH₃—CH₂— | |
| 1074 | 3-Cl-5-OCH₃—C₆H₃ | —O—CHCH₃—CH₂— | |
| 1075 | 3,5-(CH₃)₂—C₆H₃ | —O—CHCH₃—CH₂— | |
| 1076 | 3-CH₃-5-CF₃—C₆H₃ | —O—CHCH₃—CH₂— | |
| 1077 | 3-CH₃-5-OCH₃—C₆H₃ | —O—CHCH₃—CH₂— | |
| 1078 | 3,5-(CF₃)₂—C₆H₃ | —O—CHCH₃—CH₂— | |
| 1079 | 3-CF₃-5-OCH₃—C₆H₃ | —O—CHCH₃—CH₂— | |
| 1080 | 3,5-(OCH₃)₂—C₆H₃ | —O—CHCH₃—CH₂— | |
| 1081 | 2,4-F₂—C₆H₃ | —O—CHCH₃—CH₂— | |
| 1082 | 2-F-4-Cl—C₆H₃ | —O—CHCH₃—CH₂— | |
| 1083 | 2-F-4-CH₃—C₆H₃ | —O—CHCH₃—CH₂— | |
| 1084 | 2-F-4-CF₃—C₆H₃ | —O—CHCH₃—CH₂— | |
| 1085 | 2-F-4-OCH₃—C₆H₃ | —O—CHCH₃—CH₂— | |
| 1086 | 2-Cl-4-F—C₆H₃ | —O—CHCH₃—CH₂— | |
| 1087 | 2,4-Cl₂—C₆H₃ | —O—CHCH₃—CH₂— | |
| 1088 | 2-Cl-4-CH₃—C₆H₃ | —O—CHCH₃—CH₂— | |
| 1089 | 2-Cl-4-CF₃—C₆H₃ | —O—CHCH₃—CH₂— | |
| 1090 | 2-Cl-4-OCH₃—C₆H₃ | —O—CHCH₃—CH₂— | |
| 1091 | 2-CH₃-4-F—C₆H₃ | —O—CHCH₃—CH₂— | |
| 1092 | 2-CH₃-4-Cl—C₆H₃ | —O—CHCH₃—CH₂— | |
| 1093 | 2,4-(CH₃)₂—C₆H₃ | —O—CHCH₃—CH₂— | |
| 1094 | 2-CH₃-4-CF₃—C₆H₃ | —O—CHCH₃—CH₂— | |
| 1095 | 2-CH₃-4-OCH₃—C₆H₃ | —O—CHCH₃—CH₂— | |
| 1096 | 2-CF₃-4-F—C₆H₃ | —O—CHCH₃—CH₂— | |
| 1097 | 2-CF₃-4-Cl—C₆H₃ | —O—CHCH₃—CH₂— | |
| 1098 | 2-CF₃-4-CH₃—C₆H₃ | —O—CHCH₃—CH₂— | |
| 1099 | 2,4-(CF₃)₂—C₆H₃ | —O—CHCH₃—CH₂— | |
| 1100 | 2-CF₃-4-OCH₃—C₆H₃ | —O—CHCH₃—CH₂— | |
| 1101 | 2-OCH₃-4-F—C₆H₃ | —O—CHCH₃—CH₂— | |
| 1102 | 2-OCH₃-4-Cl—C₆H₃ | —O—CHCH₃—CH₂— | |
| 1103 | 2-OCH₃-4-CH₃—C₆H₃ | —O—CHCH₃—CH₂— | |
| 1104 | 2-OCH₃-4-CF₃—C₆H₃ | —O—CHCH₃—CH₂— | |
| 1105 | 2,4-(OCH₃)₂—C₆H₃ | —O—CHCH₃—CH₂— | |
| 1106 | F | —CH(CH₃)—CH(CH₃)—O— | |
| 1107 | Cl | —CH(CH₃)—CH(CH₃)—O— | |
| 1108 | CH₃ | —CH(CH₃)—CH(CH₃)—O— | |
| 1109 | C₂H₅ | —CH(CH₃)—CH(CH₃)—O— | |
| 1110 | (CH₂)₂CH₃ | —CH(CH₃)—CH(CH₃)—O— | |
| 1111 | CH(CH₃)₂ | —CH(CH₃)—CH(CH₃)—O— | |
| 1112 | (CH₂)₃CH₃ | —CH(CH₃)—CH(CH₃)—O— | |
| 1113 | CH₂CH(CH₃)₂ | —CH(CH₃)—CH(CH₃)—O— | |
| 1114 | C(CH₃)₃ | —CH(CH₃)—CH(CH₃)—O— | |
| 1115 | C₆H₅ | —CH(CH₃)—CH(CH₃)—O— | |
| 1116 | 2-F—C₆H₄ | —CH(CH₃)—CH(CH₃)—O— | |
| 1117 | 2-Cl—C₆H₄ | —CH(CH₃)—CH(CH₃)—O— | |
| 1118 | 2-CH₃—C₆H₄ | —CH(CH₃)—CH(CH₃)—O— | |
| 1119 | 2-CF₃—C₆H₄ | —CH(CH₃)—CH(CH₃)—O— | |
| 1120 | 2-OCH₃—C₆H₄ | —CH(CH₃)—CH(CH₃)—O— | |
| 1121 | 3-F—C₆H₄ | —CH(CH₃)—CH(CH₃)—O— | |
| 1122 | 3-Cl—C₆H₄ | —CH(CH₃)—CH(CH₃)—O— | |
| 1123 | 3-CH₃—C₆H₄ | —CH(CH₃)—CH(CH₃)—O— | |
| 1124 | 3-CF₃—C₆H₄ | —CH(CH₃)—CH(CH₃)—O— | |
| 1125 | 3-OCH₃—C₆H₄ | —CH(CH₃)—CH(CH₃)—O— | |
| 1126 | 4-F—C₆H₄ | —CH(CH₃)—CH(CH₃)—O— | |
| 1127 | 4-Cl—C₆H₄ | —CH(CH₃)—CH(CH₃)—O— | |
| 1128 | 4-CH₃—C₆H₄ | —CH(CH₃)—CH(CH₃)—O— | |
| 1129 | 4-CF₃—C₆H₄ | —CH(CH₃)—CH(CH₃)—O— | |
| 1130 | 4-OCH₃—C₆H₄ | —CH(CH₃)—CH(CH₃)—O— | |
| 1131 | 3,5-F₂—C₆H₃ | —CH(CH₃)—CH(CH₃)—O— | |
| 1132 | 3-F-5-Cl—C₆H₃ | —CH(CH₃)—CH(CH₃)—O— | |
| 1133 | 3-F-5-CH₃—C₆H₃ | —CH(CH₃)—CH(CH₃)—O— | |
| 1134 | 3-F-5-CF₃—C₆H₃ | —CH(CH₃)—CH(CH₃)—O— | |
| 1135 | 3-F-5-OCH₃—C₆H₃ | —CH(CH₃)—CH(CH₃)—O— | |
| 1136 | 3,5-Cl₂—C₆H₃ | —CH(CH₃)—CH(CH₃)—O— | |
| 1137 | 3-Cl-5-CH₃—C₆H₃ | —CH(CH₃)—CH(CH₃)—O— | |
| 1138 | 3-Cl-5-CF₃—C₆H₃ | —CH(CH₃)—CH(CH₃)—O— | |
| 1139 | 3-Cl-5-OCH₃—C₆H₃ | —CH(CH₃)—CH(CH₃)—O— | |
| 1140 | 3,5-(CH₃)₂—C₆H₃ | —CH(CH₃)—CH(CH₃)—O— | |
| 1141 | 3-CH₃-5-CF₃—C₆H₃ | —CH(CH₃)—CH(CH₃)—O— | |
| 1142 | 3-CH₃-5-OCH₃—C₆H₃ | —CH(CH₃)—CH(CH₃)—O— | |
| 1143 | 3,5-(CF₃)₂—C₆H₃ | —CH(CH₃)—CH(CH₃)—O— | |
| 1144 | 3-CF₃-5-OCH₃—C₆H₃ | —CH(CH₃)—CH(CH₃)—O— | |
| 1145 | 3,5-(OCH₃)₂—C₆H₃ | —CH(CH₃)—CH(CH₃)—O— | |
| 1146 | 2,4-F₂—C₆H₃ | —CH(CH₃)—CH(CH₃)—O— | |
| 1147 | 2-F-4-Cl—C₆H₃ | —CH(CH₃)—CH(CH₃)—O— | |
| 1148 | 2-F-4-CH₃—C₆H₃ | —CH(CH₃)—CH(CH₃)—O— | |
| 1149 | 2-F-4-CF₃—C₆H₃ | —CH(CH₃)—CH(CH₃)—O— | |
| 1150 | 2-F-4-OCH₃—C₆H₃ | —CH(CH₃)—CH(CH₃)—O— | |
| 1151 | 2-Cl-4-F—C₆H₃ | —CH(CH₃)—CH(CH₃)—O— | |
| 1152 | 2,4-Cl₂—C₆H₃ | —CH(CH₃)—CH(CH₃)—O— | |
| 1153 | 2-Cl-4-CH₃—C₆H₃ | —CH(CH₃)—CH(CH₃)—O— | |
| 1154 | 2-Cl-4-CF₃—C₆H₃ | —CH(CH₃)—CH(CH₃)—O— | |
| 1155 | 2-Cl-4-OCH₃—C₆H₃ | —CH(CH₃)—CH(CH₃)—O— | |
| 1156 | 2-CH₃-4-F—C₆H₃ | —CH(CH₃)—CH(CH₃)—O— | |
| 1157 | 2-CH₃-4-Cl—C₆H₃ | —CH(CH₃)—CH(CH₃)—O— | |
| 1158 | 2,4-(CH₃)₂—C₆H₃ | —CH(CH₃)—CH(CH₃)—O— | |
| 1159 | 2-CH₃-4-CF₃—C₆H₃ | —CH(CH₃)—CH(CH₃)—O— | |
| 1160 | 2-CH₃-4-OCH₃—C₆H₃ | —CH(CH₃)—CH(CH₃)—O— | |
| 1161 | 2-CF₃-4-F—C₆H₃ | —CH(CH₃)—CH(CH₃)—O— | |
| 1162 | 2-CF₃-4-Cl—C₆H₃ | —CH(CH₃)—CH(CH₃)—O— | |
| 1163 | 2-CF₃-4-CH₃—C₆H₃ | —CH(CH₃)—CH(CH₃)—O— | |
| 1164 | 2,4-(CF₃)₂—C₆H₃ | —CH(CH₃)—CH(CH₃)—O— | |
| 1165 | 2-CF₃-4-OCH₃—C₆H₃ | —CH(CH₃)—CH(CH₃)—O— | |
| 1166 | 2-OCH₃-4-F—C₆H₃ | —CH(CH₃)—CH(CH₃)—O— | |
| 1167 | 2-OCH₃-4-Cl—C₆H₃ | —CH(CH₃)—CH(CH₃)—O— | |
| 1168 | 2-OCH₃-4-CH₃—C₆H₃ | —CH(CH₃)—CH(CH₃)—O— | |
| 1169 | 2-OCH₃-4-CF₃—C₆H₃ | —CH(CH₃)—CH(CH₃)—O— | |
| 1170 | 2,4-(OCH₃)₂—C₆H₃ | —CH(CH₃)—CH(CH₃)—O— | |
| line | R⁴ | R^{Ar1} | R^{Ar2} |
| 1171 | F | Cl | Cl |
| 1172 | Cl | Cl | Cl |
| 1173 | CH₃ | Cl | Cl |
| 1174 | C₂H₅ | Cl | Cl |
| 1175 | (CH₂)₂CH₃ | Cl | Cl |
| 1176 | CH(CH₃)₂ | Cl | Cl |
| 1177 | (CH₂)₃CH₃ | Cl | Cl |
| 1178 | CH₂CH(CH₃)₂ | Cl | Cl |
| 1179 | C(CH₃)₃ | Cl | Cl |
| 1180 | C₆H₅ | Cl | Cl |
| 1181 | 2-F—C₆H₄ | Cl | Cl |
| 1182 | 2-Cl—C₆H₄ | Cl | Cl |
| 1183 | 2-CH₃—C₆H₄ | Cl | Cl |
| 1184 | 2-CF₃—C₆H₄ | Cl | Cl |
| 1185 | 2-OCH₃—C₆H₄ | Cl | Cl |
| 1186 | 3-F—C₆H₄ | Cl | Cl |
| 1187 | 3-Cl—C₆H₄ | Cl | Cl |
| 1188 | 3-CH₃—C₆H₄ | Cl | Cl |
| 1189 | 3-CF₃—C₆H₄ | Cl | Cl |
| 1190 | 3-OCH₃—C₆H₄ | Cl | Cl |
| 1191 | 4-F—C₆H₄ | Cl | Cl |
| 1192 | 4-Cl—C₆H₄ | Cl | Cl |
| 1193 | 4-CH₃—C₆H₄ | Cl | Cl |
| 1194 | 4-CF₃—C₆H₄ | Cl | Cl |
| 1195 | 4-OCH₃—C₆H₄ | Cl | Cl |
| 1196 | 3,5-F₂—C₆H₃ | Cl | Cl |
| 1197 | 3-F-5-Cl—C₆H₃ | Cl | Cl |
| 1198 | 3-F-5-CH₃—C₆H₃ | Cl | Cl |
| 1199 | 3-F-5-CF₃—C₆H₃ | Cl | Cl |
| 1200 | 3-F-5-OCH₃—C₆H₃ | Cl | Cl |
| 1201 | 3,5-Cl₂—C₆H₃ | Cl | Cl |
| 1202 | 3-Cl-5-CH₃—C₆H₃ | Cl | Cl |
| 1203 | 3-Cl-5-CF₃—C₆H₃ | Cl | Cl |
| 1204 | 3-Cl-5-OCH₃—C₆H₃ | Cl | Cl |
| 1205 | 3,5-(CH₃)₂—C₆H₃ | Cl | Cl |
| 1206 | 3-CH₃-5-CF₃—C₆H₃ | Cl | Cl |
| 1207 | 3-CH₃-5-OCH₃—C₆H₃ | Cl | Cl |
| 1208 | 3,5-(CF₃)₂—C₆H₃ | Cl | Cl |
| 1209 | 3-CF₃-5-OCH₃—C₆H₃ | Cl | Cl |
| 1210 | 3,5-(OCH₃)₂—C₆H₃ | Cl | Cl |
| 1211 | 2,4-F₂—C₆H₃ | Cl | Cl |
| 1212 | 2-F-4-Cl—C₆H₃ | Cl | Cl |
| 1213 | 2-F-4-CH₃—C₆H₃ | Cl | Cl |
| 1214 | 2-F-4-CF₃—C₆H₃ | Cl | Cl |
| 1215 | 2-F-4-OCH₃—C₆H₃ | Cl | Cl |
| 1216 | 2-Cl-4-F—C₆H₃ | Cl | Cl |
| 1217 | 2,4-Cl₂—C₆H₃ | Cl | Cl |
| 1218 | 2-Cl-4-CH₃—C₆H₃ | Cl | Cl |
| 1219 | 2-Cl-4-CF₃—C₆H₃ | Cl | Cl |
| 1220 | 2-Cl-4-OCH₃—C₆H₃ | Cl | Cl |
| 1221 | 2-CH₃-4-F—C₆H₃ | Cl | Cl |
| 1222 | 2-CH₃-4-Cl—C₆H₃ | Cl | Cl |
| 1223 | 2,4-(CH₃)₂—C₆H₃ | Cl | Cl |
| 1224 | 2-CH₃-4-CF₃—C₆H₃ | Cl | Cl |
| 1225 | 2-CH₃-4-OCH₃—C₆H₃ | Cl | Cl |
| 1226 | 2-CF₃-4-F—C₆H₃ | Cl | Cl |
| 1227 | 2-CF₃-4-Cl—C₆H₃ | Cl | Cl |
| 1228 | 2-CF₃-4-CH₃—C₆H₃ | Cl | Cl |
| 1229 | 2,4-(CF₃)₂—C₆H₃ | Cl | Cl |
| 1230 | 2-CF₃-4-OCH₃—C₆H₃ | Cl | Cl |
| 1231 | 2-OCH₃-4-F—C₆H₃ | Cl | Cl |
| 1232 | 2-OCH₃-4-Cl—C₆H₃ | Cl | Cl |
| 1233 | 2-OCH₃-4-CH₃—C₆H₃ | Cl | Cl |
| 1234 | 2-OCH₃-4-CF₃—C₆H₃ | Cl | Cl |
| 1235 | 2,4-(OCH₃)₂—C₆H₃ | Cl | Cl |
| 1236 | F | CH₃ | CH₃ |
| 1237 | Cl | CH₃ | CH₃ |
| 1238 | CH₃ | CH₃ | CH₃ |
| 1239 | C₂H₅ | CH₃ | CH₃ |
| 1240 | (CH₂)₂CH₃ | CH₃ | CH₃ |
| 1241 | CH(CH₃)₂ | CH₃ | CH₃ |
| 1242 | (CH₂)₃CH₃ | CH₃ | CH₃ |
| 1243 | CH₂CH(CH₃)₂ | CH₃ | CH₃ |
| 1244 | C(CH₃)₃ | CH₃ | CH₃ |
| 1245 | C₆H₅ | CH₃ | CH₃ |
| 1246 | 2-F—C₆H₄ | CH₃ | CH₃ |
| 1247 | 2-Cl—C₆H₄ | CH₃ | CH₃ |
| 1248 | 2-CH₃—C₆H₄ | CH₃ | CH₃ |
| 1249 | 2-CF₃—C₆H₄ | CH₃ | CH₃ |
| 1250 | 2-OCH₃—C₆H₄ | CH₃ | CH₃ |
| 1251 | 3-F—C₆H₄ | CH₃ | CH₃ |
| 1252 | 3-Cl—C₆H₄ | CH₃ | CH₃ |
| 1253 | 3-CH₃—C₆H₄ | CH₃ | CH₃ |
| 1254 | 3-CF₃—C₆H₄ | CH₃ | CH₃ |
| 1255 | 3-OCH₃—C₆H₄ | CH₃ | CH₃ |
| 1256 | 4-F—C₆H₄ | CH₃ | CH₃ |
| 1257 | 4-Cl—C₆H₄ | CH₃ | CH₃ |
| 1258 | 4-CH₃—C₆H₄ | CH₃ | CH₃ |
| 1259 | 4-CF₃—C₆H₄ | CH₃ | CH₃ |
| 1260 | 4-OCH₃—C₆H₄ | CH₃ | CH₃ |
| 1261 | 3,5-F₂—C₆H₃ | CH₃ | CH₃ |
| 1262 | 3-F-5-Cl—C₆H₃ | CH₃ | CH₃ |
| 1263 | 3-F-5-CH₃—C₆H₃ | CH₃ | CH₃ |
| 1264 | 3-F-5-CF₃—C₆H₃ | CH₃ | CH₃ |
| 1265 | 3-F-5-OCH₃—C₆H₃ | CH₃ | CH₃ |
| 1266 | 3,5-Cl₂—C₆H₃ | CH₃ | CH₃ |
| 1267 | 3-Cl-5-CH₃—C₆H₃ | CH₃ | CH₃ |
| 1268 | 3-Cl-5-CF₃—C₆H₃ | CH₃ | CH₃ |
| 1269 | 3-Cl-5-OCH₃—C₆H₃ | CH₃ | CH₃ |
| 1270 | 3,5-(CH₃)₂—C₆H₃ | CH₃ | CH₃ |
| 1271 | 3-CH₃-5-CF₃—C₆H₃ | CH₃ | CH₃ |
| 1272 | 3-CH₃-5-OCH₃—C₆H₃ | CH₃ | CH₃ |
| 1273 | 3,5-(CF₃)₂—C₆H₃ | CH₃ | CH₃ |
| 1274 | 3-CF₃-5-OCH₃—C₆H₃ | CH₃ | CH₃ |
| 1275 | 3,5-(OCH₃)₂—C₆H₃ | CH₃ | CH₃ |
| 1276 | 2,4-F₂—C₆H₃ | CH₃ | CH₃ |
| 1277 | 2-F-4-Cl—C₆H₃ | CH₃ | CH₃ |
| 1278 | 2-F-4-CH₃—C₆H₃ | CH₃ | CH₃ |
| 1279 | 2-F-4-CF₃—C₆H₃ | CH₃ | CH₃ |
| 1280 | 2-F-4-OCH₃—C₆H₃ | CH₃ | CH₃ |
| 1281 | 2-Cl-4-F—C₆H₃ | CH₃ | CH₃ |
| 1282 | 2,4-Cl₂—C₆H₃ | CH₃ | CH₃ |
| 1283 | 2-Cl-4-CH₃—C₆H₃ | CH₃ | CH₃ |
| 1284 | 2-Cl-4-CF₃—C₆H₃ | CH₃ | CH₃ |
| 1285 | 2-Cl-4-OCH₃—C₆H₃ | CH₃ | CH₃ |
| 1286 | 2-CH₃-4-F—C₆H₃ | CH₃ | CH₃ |
| 1287 | 2-CH₃-4-Cl—C₆H₃ | CH₃ | CH₃ |
| 1288 | 2,4-(CH₃)₂—C₆H₃ | CH₃ | CH₃ |
| 1289 | 2-CH₃-4-CF₃—C₆H₃ | CH₃ | CH₃ |
| 1290 | 2-CH₃-4-OCH₃—C₆H₃ | CH₃ | CH₃ |
| 1291 | 2-CF₃-4-F—C₆H₃ | CH₃ | CH₃ |
| 1292 | 2-CF₃-4-Cl—C₆H₃ | CH₃ | CH₃ |
| 1293 | 2-CF₃-4-CH₃—C₆H₃ | CH₃ | CH₃ |
| 1294 | 2,4-(CF₃)₂—C₆H₃ | CH₃ | CH₃ |
| 1295 | 2-CF₃-4-OCH₃—C₆H₃ | CH₃ | CH₃ |
| 1296 | 2-OCH₃-4-F—C₆H₃ | CH₃ | CH₃ |
| 1297 | 2-OCH₃-4-Cl—C₆H₃ | CH₃ | CH₃ |
| 1298 | 2-OCH₃-4-CH₃—C₆H₃ | CH₃ | CH₃ |
| 1299 | 2-OCH₃-4-CF₃—C₆H₃ | CH₃ | CH₃ |
| 1300 | 2,4-(OCH₃)₂—C₆H₃ | CH₃ | CH₃ |

TABLE B
Examples of preferred compounds of formula II
are the compounds of formula II.a, II.b, II.c, II.d, and II.e,

(II.a)

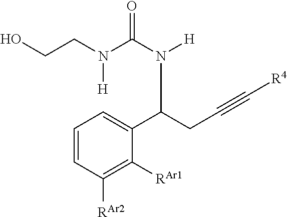

(II.b)

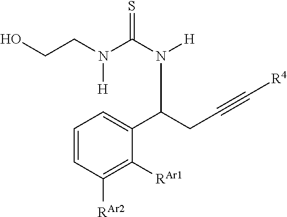

(II.c)

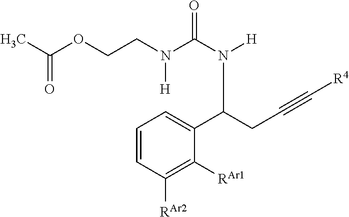

(II.d)

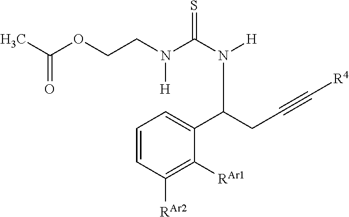

(II.e)

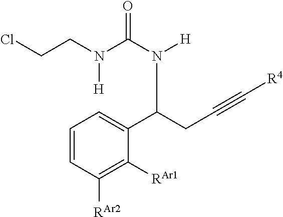

wherein R⁴, R^{Ar1} and R^{Ar2} have the meanings corresponding to one line of table 1 (compounds II.a.1 to II.a.1300, II.b.1 to II.b.1300, II.c.1 to II.c.1300, II.d.1 to II.d.1300 and II.e.1 to II.e.1300.

The compounds of formulae I and II can be obtained for example by the methods outlined in schemes 1 to 8.

Compounds of the formula I wherein R⁵ and R⁶ both have a meaning different from hydrogen, can be obtained as outlined in scheme 1.

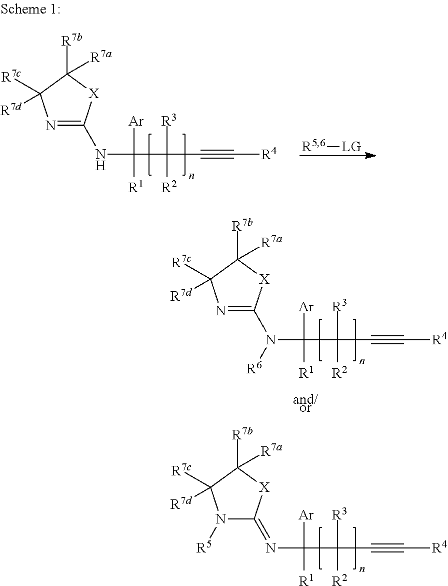

In scheme 1, R¹, R², R³, R⁴, R⁵, R⁶, R^{7a}, R^{7b}, R^{7c}, R^{7d} and Ar have one of the meanings given above. A compound of formula I wherein R⁵ or R⁶ is hydrogen is treated with a suitable electrophile. Suitable electrophiles are e.g. alkylating or acylating agents R^{5,6}-LG (LG=leaving group; e.g. CI, Br, I, OSO₂R, OCO₂R, wherein the R is C₁-C₄-alkyl) as described e.g. in WO 2005063724.

The compounds of the formula I wherein X is oxygen or sulfur can be prepared e.g. from the corresponding aminothiocarbonylaminoethanol compounds of formula II.a or from aminocarbonylaminoethanol compounds of formula II.b, respectively, as depicted in scheme 2.

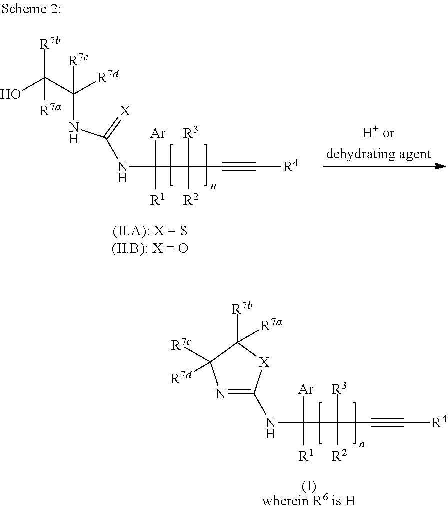

In scheme 2, R¹, R², R³, R⁴, R^{7a}, R^{7b}, R^{7c}, R^{7d} and Ar are as defined above.

The aminothiocarbonylaminoethanol compound II.A and the aminocarbonylaminoethanol compound II.B, respectively, can be cyclized by conventional means thereby obtaining an azoline compound of the formula I. Cyclization of the compounds II.A and II.B, respectively, can be achieved e.g. under acid catalysis or under dehydrating conditions e.g. by Mitsunobu's reaction (see Tetrahedron Letters 1999, 40, 3125-3128) or as described in the preparation example 13.

Compounds of the formula II.A and II.B, respectively, can be prepared as depicted in schemes 3 and 4 below.

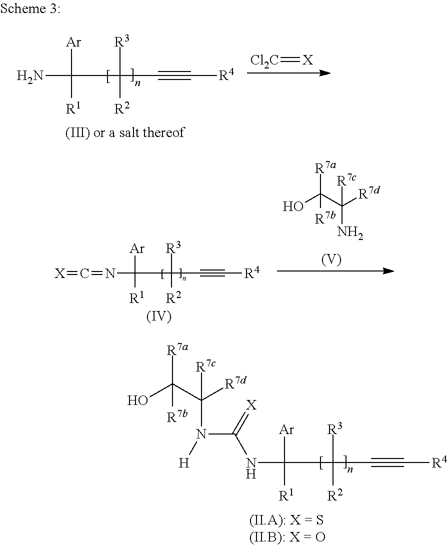

In scheme 3, R¹, R², R³, R⁴, R^{7a}, R^{7b}, R^{7c}, R^{7d} and Ar are as defined above. An amine III or a salt thereof is converted to the corresponding iso(thio)cyanate IV by conventional means, e.g. by reacting III with (thio)phosgene, as described for example in the case of thiophosgene in Houben-Weyl, E4, “Methoden der Organischen Chemie”, chapter IIc, pp. 837-842, Georg Thieme Verlag 1983. It may be advantageous to carry out the reaction in the presence of a base. The iso(thio)cyanate IV is then reacted with an aminoethanol V to form an amino(thio)carbonylaminoethanol compound. The reaction of the aminoethanol V with iso(thio)cyanate IV can be performed in accordance with standard methods of organic chemistry, see e.g. Biosci. Biotech. Biochem. 56 (7), 1062-65 (1992).

A further route to compounds II, wherein X is S is shown in scheme 4.

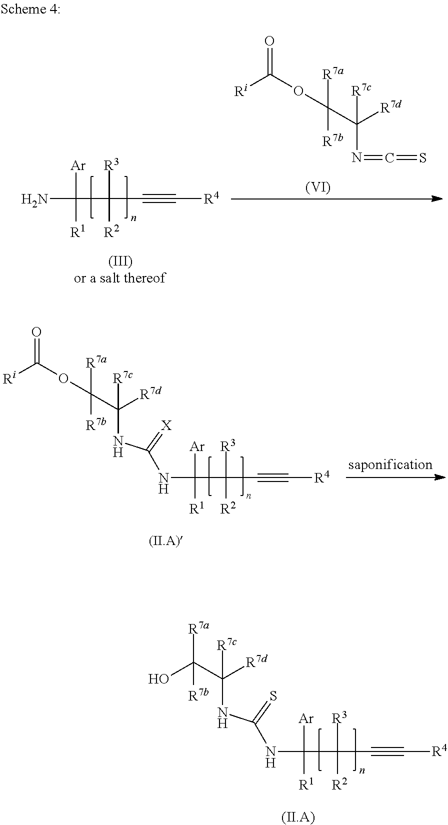

In scheme 4, R¹, R², R³, R⁴, R^{7a}, R^{7b}, R^{7c}, R^{7d}, Rⁱ and Ar are as defined above. An aminoalkynyl compound III or a salt thereof can be converted to the corresponding aminothiocarbonylaminoethanol compound II.A, by reaction of the compound III with an isothiocyanate VI and subsequent saponification as described in the preparation examples 1 and 2. Suitable isothiocyanates can be prepared according to the procedures described in Coll. Czech. Chem. Commun. 1986, 51, 112-117.

Aminoalkynyl compounds of formula III are known in the art or can be prepared by methods familiar to an organic chemist, for instance by application of general methods for the synthesis of amines and alkynes and as depicted e.g. in scheme 5 or shown in the preparation examples 23 and 24. Suitable salts of aminoalkynyl compounds III are e.g. the acid addition salts formed by treating a compound of formula III with an inorganic or organic acid. Anions of useful acids are e.g. sulfate, hydrogensulfate, phosphate, dihydrogenphosphate, hydrogenphosphate, nitrate, bicarbonate, carbonate, chloride, bromide, p-toluene sulfonate, and the anions of C₁-C₄-alkanoic acids such as acetate, propionate, and the like.

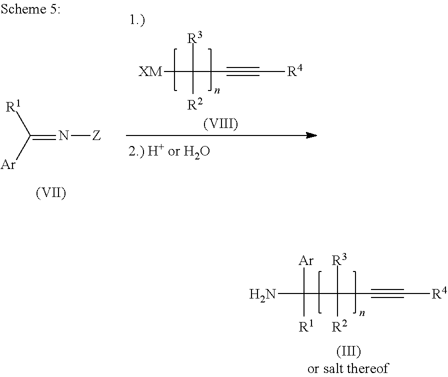

In scheme 5, R¹, R², R³, R⁴, and Ar are as defined above, Z is for example SiMe₃ or S(═O)-tert-butyl, X is a halogen and M is for example Zn, Mg, etc.

The addition of metal halides VIII to appropriate imines VII followed by aqueous work up or acidic treatment can provide amines III for example as described in Org. Biomol. Chem. 2004, 2, 3060-3062 or Chem. Lett. 1995, 3, 235-236 or the preparation example 23.

Compounds of formula III wherein R⁴ is hydrogen can be used for further derivatisation of the alkyne unit by cross coupling reactions such as Sonogashira for example like in Chem. Rev. 2007, 107, 874-922 or the preparation example 15.

Alternatively, the compounds of the formula I according to the invention wherein X is O or S can be prepared by the method shown in scheme 6.

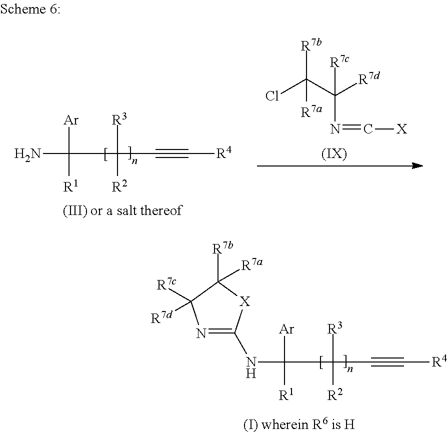

In scheme 6, R¹, R², R³, R⁴, R^{7a}, R^{7b}, R^{7c}, R^{7d} and Ar are as defined above.

An amine of formula III or a salt thereof can be converted to an azoline of formula I by reaction with a 2-chloroethylisothiocyanate or a 2-chloroethylisocyanate of formula IX e.g. as described in Bioorg. Med. Chem. Lett. 1994, 4, 2317-22 and subsequent cyclization in the presence or absence of base, as in the preparation example 14. 1-Chloro-2-isothiocyanatoethane (CAS-reg.-no.: 6099-88-3) and 2-chloro-ethyl-isocyanate (CAS-reg.-no.: 1943-83-5) are commercially available.

Compounds of the formula I wherein X is NR⁸ may be prepared by the method shown in scheme 7.

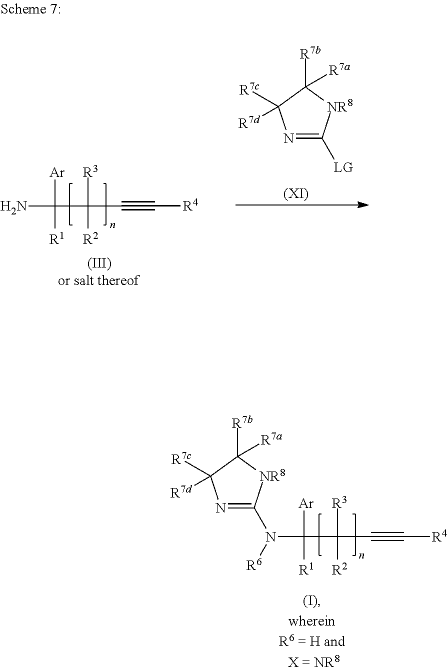

In scheme 7, R¹, R², R³, R⁴, R⁶, R^{7a}, R^{7b}, R^{7c}, R^{7d}, R⁸ and Ar are as defined above and LG is a leaving group.

Compounds of the formula I may be obtained by reacting an appropriate substituted amine III or a salt thereof with a 2-substituted imidazoline XI in an appropriate solvent. This reaction can be carried out, for example by analogy to the methods described in U.S. Pat. No. 5,130,441 or EP 0389765.

Alternatively, the compounds of formula I, wherein X is oxygen or sulfur and R¹ is hydrogen, can also be prepared starting from amines XII via aminothiazolines or aminooxazolines XIII, as outlined in scheme 9. A further conversion with an alkylating or acylating reagent R^{8,9}-LG can provide derivatised aminothiazolines or aminooxazolines XIV and/or XV. Those can be subsequently alkylated with electrophiles of formula XVI, optionally followed by hydrolysis, as outlined in scheme 8.

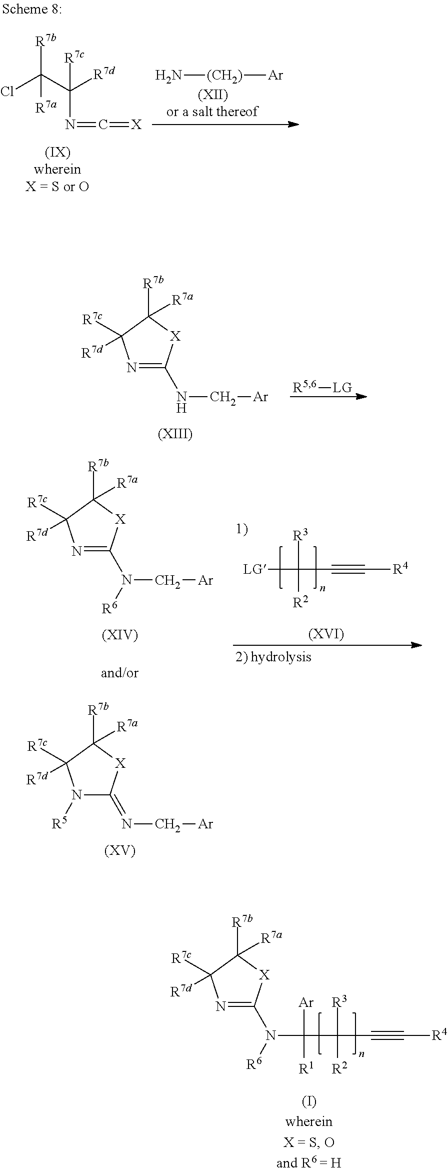

In scheme 8, R², R³, R⁴, R⁵, R⁶, R^{7a}, R^{7b}, R^{7c}, R^{7d} and Ar are as defined above and LG and LG′ are leaving groups, such as halogen.

As a rule, the compounds of formulae I and II can be prepared by the methods described above. If individual compounds cannot be prepared via the above-described routes, they can be prepared by derivatization of other compounds of formulae I and II or by customary modifications of the synthesis routes described. For example, in individual cases, certain compounds of formulae I and II can advantageously be prepared from other compounds of formula I by ester hydrolysis, amidation, esterification, ether cleavage, olefination, reduction, metal catalyzed coupling reactions, oxidation and the like.

The reaction mixtures are worked up in the customary manner, for example by mixing with water, separating the phases, and, if appropriate, purifying the crude products by chromatography, for example on alumina or on silica gel. Some of the intermediates and end products may be obtained in the form of colourless or pale brown viscous oils which are freed or purified from volatile components under reduced pressure and at moderately elevated temperature. If the intermediates and end products are obtained as solids, they may be purified by recrystallization or digestion.

The inventive compounds of formula I or III may be present in different crystalline modifications which may differ in their biological activity. These are also subject of the present invention.

The compounds of the formulae I and II and the pestidicidal compositions comprising them are effective agents for controlling arthropod pests and nematodes.

### Pests

The compounds of the formula I and formula II, and their salts are in particular suitable for efficiently controlling arthropodal pests such as arachnids, myriapedes and insects as well as nematodes.

Animal pests controlled by the compounds of formulae I and II include for example:

Insects from the order of the lepidopterans (Lepidoptera), for example Agrotis ypsilon, Agrotis segetum, Alabama argillacea, Anticarsia gemmatalis, Argyresthia conjugella, Autographa gamma, Bupalus piniarius, Cacoecia murinana, Capua reticulana, Chematobia brumata, Choristoneura fumiferana, Choristoneura occidentalis, Cirphis unipuncta, Cydia pomonella, Dendrolimus pini, Diaphania nitidalis, Diatraea grandiosella, Earias insulana, Elasmopalpus lignosellus, Eupoecilia ambiguella, Evetria bouliana, Feltia subterranea, Galleria mellonella, Grapholitha funebrana, Grapholitha molesta, Heliothis armigera, Heliothis virescens, Heliothis zea, Hellula undalis, Hibernia defoliaria, Hyphantria cunea, Hyponomeuta malinellus, Keiferia lycopersicella, Lambdina fiscellaria, Laphygma exigua, Leucoptera coffeella, Leucoptera scitella, Lithocolletis blancardella, Lobesia botrana, Loxostege sticticalis, Lymantria dispar, Lymantria monacha, Lyonetia clerkella, Malacosoma neustria, Mamestra brassicae, Orgyia pseudotsugata, Ostrinia nubilalis, Panolis flammea, Pectinophora gossypiella, Peridroma saucia, Phalera bucephala, Phthorimaea operculella, Phyllocnistis citrella, Pieris brassicae, Plathypena scabra, Plutella xylostella, Pseudoplusia includens, Rhyacionia frustrana, Scrobipalpula absoluta, Sitotroga cerealella, Sparganothis pilleriana, Spodoptera frugiperda, Spodoptera littoralis, Spodoptera litura, Thaumatopoea pityocampa, Tortrix viridana, Trichoplusia ni and Zeiraphera canadensis;

beetles (Coleoptera), for example Agrilus sinuatus, Agriotes lineatus, Agriotes obscurus, Amphimallus solstitialis, Anisandrus dispar, Anthonomus grandis, Anthonomus pomorum, Aphthona euphoridae, Athous haemorrhoidalis, Atomaria linearis, Blastophagus piniperda, Blitophaga undata, Bruchus rufimanus, Bruchus pisorum, Bruchus lentis, Byctiscus betulae, Cassida nebulosa, Cerotoma trifurcata, Cetonia aurata, Ceuthorrhynchus assimilis, Ceuthorrhynchus napi, Chaetocnema tibialis, Conoderus vespertinus, Crioceris asparagi, Ctenicera ssp., Diabrotica longicornis, Diabrotica semipunctata, Diabrotica 12-punctata Diabrotica speciosa, Diabrotica virgifera, Epilachna varivestis, Epitrix hirtipennis, Eutinobothrus brasiliensis, Hylobius abietis, Hypera brunneipennis, Hypera postica, Ips typographus, Lema bilineata, Lema melanopus, Leptinotarsa decemlineata, Limonius californicus, Lissorhoptrus oryzophilus, Melanotus communis, Meligethes aeneus, Melolontha hippocastani, Melolontha melolontha, Oulema oryzae, Otiorrhynchus sulcatus, Otiorrhynchus ovatus, Phaedon cochleariae, Phyllobius pyri, Phyllotreta chrysocephala, Phyllophaga sp., Phyllopertha horticola, Phyllotreta nemorum, Phyllotreta striolata, Popillia japonica, Sitona lineatus and Sitophilus granaria;
flies, mosquitoes (Diptera), e.g. Aedes aegypti, Aedes albopictus, Aedes vexans, Anastrepha ludens, Anopheles maculipennis, Anopheles crucians, Anopheles albimanus, Anopheles gambiae, Anopheles freeborni, Anopheles leucosphyrus, Anopheles minimus, Anopheles quadrimaculatus, Calliphora vicina, Ceratitis capitata, Chrysomya bezziana, Chrysomya hominivorax, Chrysomya macellaria, Chrysops discalis, Chrysops silacea, Chrysops atlanticus, Cochliomyia hominivorax, Contarinia sorghicola Cordylobia anthropophaga, Culicoides furens, Culex pipiens, Culex nigripalpus, Culex quinquefasciatus, Culex tarsalis, Culiseta inornata, Culiseta melanura, Dacus cucurbitae, Dacus oleae, Dasineura brassicae, Delia antique, Delia coarctata, Delia platura, Delia radicum, Dermatobia hominis, Fannia canicularis, Geomyza Tripunctata, Gasterophilus intestinalis, Glossina morsitans, Glossina palpalis, Glossina fuscipes, Glossina tachinoides, Haematobia irritans, Haplodiplosis equestris, Hippelates spp., Hylemyia platura, Hypoderma lineata, Leptoconops torrens, Liriomyza sativae, Liriomyza trifolii, Lucilia caprina, Lucilia cuprina, Lucilia sericata, Lycoria pectoralis, Mansonia titillanus, Mayetiola destructor, Musca autumnalis, Musca domestica, Muscina stabulans, Oestrus ovis, Opomyza florum, Oscinella frit, Pegomya hysocyami, Phorbia antiqua, Phorbia brassicae, Phorbia coarctata, Phlebotomus argentipes, Psorophora columbiae, Psila rosae, Psorophora discolor, Prosimulium mixtum, Rhagoletis cerasi, Rhagoletis pomonella, Sarcophaga haemorrhoidalis, Sarcophaga spp., Simulium vittatum, Stomoxys calcitrans, Tabanus bovinus, Tabanus atratus, Tabanus lineola, and Tabanus similis, Tipula oleracea, and Tipula paludosa;
thrips (Thysanoptera), e.g. Dichromothrips corbetti, Dichromothrips ssp., Frankliniella fusca, Frankliniella occidentalis, Frankliniella tritici, Scirtothrips citri, Thrips oryzae, Thrips palmi and Thrips tabaci,
termites (Isoptera), e.g. Calotermes flavicollis, Leucotermes flavipes, Heterotermes aureus, Reticulitermes flavipes, Reticulitermes virginicus, Reticulitermes lucifugus, Reticulitermes santonensis, Reticulitermes grassei, Termes natalensis, and Coptotermes formosanus;
cockroaches (Blattaria—Blattodea), e.g. Blattella germanica, Blattella asahinae, Periplaneta americana, Periplaneta japonica, Periplaneta brunnea, Periplaneta fuligginosa, Periplaneta australasiae, and Blatta orientalis;
bugs, aphids, leafhoppers, whiteflies, scale insects, cicadas (Hemiptera), e.g. Acrosternum hilare, Blissus leucopterus, Cyrtopeltis notatus, Dysdercus cingulatus, Dysdercus intermedius, Eurygaster integriceps, Euschistus impictiventris, Leptoglossus phyllopus, Lygus lineolaris, Lygus pratensis, Nezara viridula, Piesma quadrata, Solubea insularis, Thyanta perditor, Acyrthosiphon onobrychis, Adelges laricis, Aphidula nasturtii, Aphis fabae, Aphis forbesi, Aphis pomi, Aphis gossypii, Aphis grossulariae, Aphis schneideri, Aphis spiraecola, Aphis sambuci, Acyrthosiphon pisum, Aulacorthum solani, Bemisia argentifolii, Brachycaudus cardui, Brachycaudus helichrysi, Brachycaudus persicae, Brachycaudus prunicola, Brevicoryne brassicae, Capitophorus horni, Cerosipha gossypii, Chaetosiphon fragaefolii, Cryptomyzus ribis, Dreyfusia nordmannianae, Dreyfusia piceae, Dysaphis radicola, Dysaulacorthum pseudosolani, Dysaphis plantaginea, Dysaphis pyri, Empoasca fabae, Hyalopterus pruni, Hyperomyzus lactucae, Macrosiphum avenae, Macrosiphum euphorbiae, Macrosiphon rosae, Megoura viciae, Melanaphis pyrarius, Metopolophium dirhodum, Myzus persicae, Myzus ascalonicus, Myzus cerasi, Myzus varians, Nasonovia ribis-nigri, Nilaparvata lugens, Pemphigus bursarius, Perkinsiella saccharicida, Phorodon humuli, Psylla mali, Psylla pin, Rhopalomyzus ascalonicus, Rhopalosiphum maidis, Rhopalosiphum padi, Rhopalosiphum insertum, Sappaphis mala, Sappaphis mali, Schizaphis graminum, Schizoneura lanuginosa, Sitobion avenae, Trialeurodes vaporariorum, Toxoptera aurantiiand, Viteus vitifolii, Cimex lectularius, Cimex hemipterus, Reduvius senilis, Triatoma spp., and Arilus critatus;
ants, bees, wasps, sawflies (Hymenoptera), e.g. Athalia rosae, Atta cephalotes, Atta capiguara, Atta cephalotes, Atta laevigata, Atta robusta, Atta sexdens, Atta texana, Crematogaster spp., Hoplocampa minuta, Hoplocampa testudinea, Lasius niger, Monomorium pharaonis, Solenopsis geminata, Solenopsis invicta, Solenopsis richteri, Solenopsis xyloni, Pogonomyrmex barbatus, Pogonomyrmex californicus, Pheidole megacephala, Dasymutilla occidentalis, Bombus spp., Vespula squamosa, Paravespula vulgaris, Paravespula pennsylvanica, Paravespula germanica, Dolichovespula maculata, Vespa crabro, Polistes rubiginosa, Camponotus floridanus, and Linepithema humile;
crickets, grasshoppers, locusts (Orthoptera), e.g. Acheta domestica, Gryllotalpa gryllotalpa, Locusta migratoria, Melanoplus bivittatus, Melanoplus femurrubrum, Melanoplus mexicanus, Melanoplus sanguinipes, Melanoplus spretus, Nomadacris septemfasciata, Schistocerca americana, Schistocerca gregaria, Dociostaurus maroccanus, Tachycines asynamorus, Oedaleus senegalensis, Zonozerus variegatus, Hieroglyphus daganensis, Kraussaria angulifera, Calliptamus italicus, Chortoicetes terminifera, and Locustana pardalina;
arachnoidea, such as arachnids (Acarina), e.g. of the families Argasidae, Ixodidae and Sarcoptidae, such as Amblyomma americanum, Amblyomma variegatum, Ambryomma maculatum, Argas persicus, Boophilus annulatus, Boophilus decoloratus, Boophilus microplus, Dermacentor silvarum, Dermacentor andersoni, Dermacentor variabilis, Hyalomma truncatum, Ixodes ricinus, Ixodes rubicundus, Ixodes scapularis, Ixodes holocyclus, Ixodes pacificus, Ornithodorus moubata, Ornithodorus hermsi, Ornithodorus turicata, Ornithonyssus bacoti, Otobius megnini, Dermanyssus gallinae, Psoroptes ovis, Rhipicephalus sanguineus, Rhipicephalus appendiculatus, Rhipicephalus evertsi, Sarcoptes scabiei, and Eriophyidae spp. such as Aculus schlechtendali, Phyllocoptrata oleivora and Eriophyes sheldoni; Tarsonemidae spp. such as Phytonemus pallidus and Polyphagotarsonemus latus; Tenuipalpidae spp. such as Brevipalpus phoenicis; Tetranychidae spp. such as Tetranychus cinnabarinus, Tetranychus kanzawai, Tetranychus pacificus, Tetranychus telarius and Tetranychus urticae, Panonychus ulmi, Panonychus citri, and Oligonychus pratensis; Araneida, e.g. Latrodectus mactans, and Loxosceles reclusa;
fleas (Siphonaptera), e.g. Ctenocephalides felis, Ctenocephalides canis, Xenopsylla cheopis, Pulex irritans, Tunga penetrans, and Nosopsyllus fasciatus,

silverfish, firebrat (Thysanura), e.g. Lepisma saccharina and Thermobia domestica,

centipedes (Chilopoda), e.g. Scutigera coleoptrata,

millipedes (Diplopoda), e.g. Narceus spp.,

Earwigs (Dermaptera), e.g. forficula auricularia,

lice (Phthiraptera), e.g. Pediculus humanus capitis, Pediculus humanus corporis, Pthirus pubis, Haematopinus eurysternus, Haematopinus suis, Linognathus vituli, Bovicola bovis, Menopon gallinae, Menacanthus stramineus and Solenopotes capillatus. Collembola (springtails), e.g. Onychiurus ssp.

They are also suitable for controlling Nematodes: plant parasitic nematodes such as root knot nematodes, Meloidogyne hapla, Meloidogyne incognita, Meloidogyne javanica, and other Meloidogyne species; cyst-forming nematodes, Globodera rostochiensis and other Globodera species; Heterodera avenae, Heterodera glycines, Heterodera schachtii, Heterodera trifolii, and other Heterodera species; Seed gall nematodes, Anguina species; Stem and foliar nematodes, Aphelenchoides species; Sting nematodes, Belonolaimus longicaudatus and other Belonolaimus species; Pine nematodes, Bursaphelenchus xylophilus and other Bursaphelenchus species; Ring nematodes, Criconema species, Criconemella species, Criconemoides species, Mesocriconema species; Stem and bulb nematodes, Ditylenchus destructor, Ditylenchus dipsaci and other Ditylenchus species; Awl nematodes, Dolichodorus species; Spiral nematodes, Heliocotylenchus multicinctus and other Helicotylenchus species; Sheath and sheathoid nematodes, Hemicycliophora species and Hemicriconemoides species; Hirshmanniella species; Lance nematodes, Hoploaimus species; false rootknot nematodes, Nacobbus species; Needle nematodes, Longidorus elongatus and other Longidorus species; Lesion nematodes, Pratylenchus neglectus, Pratylenchus penetrans, Pratylenchus curvitatus, Pratylenchus goodeyi and other Pratylenchus species; Burrowing nematodes, Radopholus similis and other Radopholus species; Reniform nematodes, Rotylenchus robustus and other Rotylenchus species; Scutellonema species; Stubby root nematodes, Trichodorus primitivus and other Trichodorus species, Paratrichodorus species; Stunt nematodes, Tylenchorhynchus claytoni, Tylenchorhynchus dubius and other Tylenchorhynchus species; Citrus nematodes, Tylenchulus species; Dagger nematodes, Xiphinema species; and other plant parasitic nematode species.

The compounds of the formula I and II and their salts are also useful for controlling arachnids (Arachnoidea), such as acarians (Acarina), e.g. of the families Argasidae, Ixodidae and Sarcoptidae, such as Amblyomma americanum, Amblyomma variegatum, Argas persicus, Boophilus annulatus, Boophilus decoloratus, Boophilus microplus, Dermacentor silvarum, Hyalomma truncatum, Ixodes ricinus, Ixodes rubicundus, Ornithodorus moubata, Otobius megnini, Dermanyssus gallinae, Psoroptes ovis, Rhipicephalus appendiculatus, Rhipicephalus evertsi, Sarcoptes scabiei, and Eriophyidae spp. such as Aculus schlechtendali, Phyllocoptrata oleivora and Eriophyes sheldoni; Tarsonemidae spp. such as Phytonemus pallidus and Polyphagotarsonemus latus; Tenuipalpidae spp. such as Brevipalpus phoenicis; Tetranychidae spp. such as Tetranychus cinnabarinus, Tetranychus kanzawai, Tetranychus pacificus, Tetranychus telarius and Tetranychus urticae, Panonychus ulmi, Panonychus citri, and oligonychus pratensis.

Compounds of the formula I and II are particularly useful for controlling insects, preferably sucking or piercing insects such as insects from the genera Thysanoptera, Diptera and Hemiptera, in particular the following species:

Thysanoptera: Frankliniella fusca, Frankliniella occidentalis, Frankliniella tritici, Scirtothrips citri, Thrips oryzae, Thrips palmi and Thrips tabaci,

Diptera, e.g. Aedes aegypti, Aedes albopictus, Aedes vexans, Anastrepha ludens, Anopheles maculipennis, Anopheles crucians, Anopheles albimanus, Anopheles gambiae, Anopheles freeborni, Anopheles leucosphyrus, Anopheles minimus, Anopheles quadrimaculatus, Calliphora vicina, Ceratitis capitata, Chrysomya bezziana, Chrysomya hominivorax, Chrysomya macellaria, Chrysops discalis, Chrysops silacea, Chrysops atlanticus, Cochliomyia hominivorax, Contarinia sorghicola, Cordylobia anthropophaga, Culicoides furens, Culex pipiens, Culex nigripalpus, Culex quinquefasciatus, Culex tarsalis, Culiseta inornata, Culiseta melanura, Dacus cucurbitae, Dacus oleae, Dasineura brassicae, Delia antique, Delia coarctata, Delia platura, Delia radicum, Dermatobia hominis, Fannia canicularis, Geomyza Tripunctata, Gasterophilus intestinalis, Glossina morsitans, Glossina palpalis, Glossina fuscipes, Glossina tachinoides, Haematobia irritans, Haplodiplosis equestris, Hippelates spp., Hylemyia platura, Hypoderma lineata, Leptoconops torrens, Liriomyza sativae, Liriomyza trifolii, Lucilia caprina, Lucilia cuprina, Lucilia sericata, Lycoria pectoralis, Mansonia titillanus, Mayetiola destructor, Musca autumnalis, Musca domestica, Muscina stabulans, Oestrus ovis, Opomyza florum, Oscinella frit, Pegomya hysocyami, Phorbia antiqua, Phorbia brassicae, Phorbia coarctata, Phlebotomus argentipes, Psorophora columbiae, Psila rosae, Psorophora discolor, Prosimulium mixtum, Rhagoletis cerasi, Rhagoletis pornonella, Sarcophaga haemorrhoidalis, Sarcophaga spp., Simulium vittatum, Stomoxys calcitrans, Tabanus bovinus, Tabanus atratus, Tabanus lineola, and Tabanus similis, Tipula oleracea, and Tipula paludosa;

Hemiptera, in particular aphids: Acyrthosiphon onobrychis, Adelges laricis, Aphidula nasturtii, Aphis fabae, Aphis forbesi, Aphis pomi, Aphis gossypii, Aphis grossulariae, Aphis schneideri, Aphis spiraecola, Aphis sambuci, Acyrthosiphon pisum, Aulacorthum solani, Brachycaudus cardui, Brachycaudus helichrysi, Brachycaudus persicae, Brachycaudus prunicola, Brevicoryne brassicae, Capitophorus horni, Cerosipha gossypii, Chaetosiphon fragaefolii, Cryptomyzus ribis, Dreyfusia nordmannianae, Dreyfusia piceae, Dysaphis radicola, Dysaulacorthum pseudosolani, Dysaphis plantaginea, Dysaphis pyri, Empoasca fabae, Hyalopterus pruni, Hyperomyzus lactucae, Macrosiphum avenae, Macrosiphum euphorbiae, Macrosiphon rosae, Megoura viciae, Melanaphis pyrarius, Metopolophium dirhodum, Myzodes persicae, Myzus ascalonicus, Myzus cerasi, Myzus varians, Nasonovia ribis-nigri, Nilaparvata lugens, Pemphigus bursarius, Perkinsiella saccharicida, Phorodon humuli, Psylla mali, Psylla piri, Rhopalomyzus ascalonicus, Rhopalosiphum maidis, Rhopalosiphum padi, Rhopalosiphum insertum, Sappaphis mala, Sappaphis mali, Schizaphis graminum, Schizoneura lanuginosa, Sitobion avenae, Trialeurodes vaporariorum, Toxoptera aurantiiand, and Viteus vitifolii;

Compounds of the formula I and II are particularly useful for controlling insects of the orders Hemiptera and Thysanoptera.

### Formulations

For use in a method according to the present invention, the compounds of formula I or II can be converted into the customary formulations, e.g. solutions, emulsions, suspensions, dusts, powders, pastes, granules and directly sprayable solutions. The use form depends on the particular purpose and application method. Formulations and application methods are chosen to ensure in each case a fine and uniform distribution of the compound of the formula I according to the present invention.

The formulations are prepared in a known manner (see e.g. for review U.S. Pat. No. 3,060,084, EP-A 707 445 (for liquid concentrates), Browning, “Agglomeration”, Chemical Engineering, Dec. 4, 1967, 147-48, Perry's Chemical Engineer's Handbook, 4th Ed., McGraw-Hill, New York, 1963, pages 8-57 and et seq. WO 91/13546, U.S. Pat. No. 4,172,714, U.S. Pat. No. 4,144,050, U.S. Pat. No. 3,920,442, U.S. Pat. No. 5,180,587, U.S. Pat. No. 5,232,701, U.S. Pat. No. 5,208,030, GB 2,095,558, U.S. Pat. No. 3,299,566, Klingman, Weed Control as a Science, John Wiley and Sons, Inc., New York, 1961, Hance et al., Weed Control Handbook, 8th Ed., Blackwell Scientific Publications, Oxford, 1989 and Mollet, H., Grubemann, A., Formulation technology, Wiley VCH Verlag GmbH, Weinheim (Germany), 2001, 2. D. A. Knowles, Chemistry and Technology of Agrochemical Formulations, Kluwer Academic Publishers, Dordrecht, 1998 (ISBN 0-7514-0443-8), for example by extending the active compound with auxiliaries suitable for the formulation of agrochemicals, such as solvents and/or carriers, if desired emulsifiers, surfactants and dispersants, preservatives, anti-foaming agents, anti-freezing agents, for seed treatment formulation also optionally colorants and/or binders and/or gelling agents.

Solvents/carriers, which are suitable, are e.g.:
solvents such as water, aromatic solvents (for example Solvesso products, xylene and the like), paraffins (for example mineral fractions), alcohols (for example methanol, butanol, pentanol, benzyl alcohol), ketones (for example cyclohexanone, gamma-butyrolactone), pyrrolidones (N-methyl-pyrrolidone (NMP), N-octylpyrrolidone NOP), acetates (glycol diacetate), alkyl lactates, lactones such as g-butyrolactone, glycols, fatty acid dimethylamides, fatty acids and fatty acid esters, triglycerides, oils of vegetable or animal origin and modified oils such as alkylated plant oils. In principle, solvent mixtures may also be used.
carriers such as ground natural minerals and ground synthetic minerals, such as silica gels, finely divided silicic acid, silicates, talc, kaolin, attaclay, limestone, lime, chalk, bole, loess, clay, dolomite, diatomaceous earth, calcium sulfate and magnesium sulfate, magnesium oxide, ground synthetic materials, fertilizers, such as, for example, ammonium sulfate, ammonium phosphate, ammonium nitrate, ureas and products of vegetable origin, such as cereal meal, tree bark meal, wood meal and nutshell meal, cellulose powders and other solid carriers.

Suitable emulsifiers are nonionic and anionic emulsifiers (for example polyoxyethylene fatty alcohol ethers, alkylsulfonates and arylsulfonates).

Examples of dispersants are lignin-sulfite waste liquors and methylcellulose.

Suitable surfactants are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenyl ether, ethoxylated isooctylphenol, octylphenol, nonylphenol, alkylphenyl polyglycol ethers, tributylphenyl polyglycol ether, tristearylphenyl polyglycol ether, alkylaryl polyether alcohols, alcohol and fatty alcohol/ethylene oxide condensates, ethoxylated castor oil, polyoxyethylene alkyl ethers, ethoxylated polyoxypropylene, lauryl alcohol polyglycol ether acetal, sorbitol esters,

Also anti-freezing agents such as glycerin, ethylene glycol, propylene glycol and bactericides such as can be added to the formulation.

Suitable antifoaming agents are for example antifoaming agents based on silicon or magnesium stearate.

Suitable preservatives are for example dichlorophen and benzyl alcohol hemiformal

Suitable thickeners are compounds which confer a pseudoplastic flow behavior to the formulation, i.e. high viscosity at rest and low viscosity in the agitated stage. Mention may be made, in this context, for example, of commercial thickeners based on poly-saccharides, such as Xanthan Gum® (Kelzan® from Kelco), Rhodopol®23 (Rhone Poulenc) or Veegum® (from R.T. Vanderbilt), or organic phyllosilicates, such as Attaclay® (from Engelhardt). Antifoam agents suitable for the dispersions according to the invention are, for example, silicone emulsions (such as, for example, Silikon® SRE, Wacker or Rhodorsil® from Rhodia), long-chain alcohols, fatty acids, organofluorine compounds and mixtures thereof. Biocides can be added to stabilize the compositions according to the invention against attack by microorganisms. Suitable biocides are, for example, based on isothiazolones such as the compounds marketed under the trademarks Proxel® from Avecia (or Arch) or Acticide® RS from Thor Chemie and Kathon® MK from Rohm & Haas. Suitable antifreeze agents are organic polyols, for example ethylene glycol, propylene glycol or glycerol. These are usually employed in amounts of not more than 10% by weight, based on the total weight of the active compound composition. If appropriate, the active compound compositions according to the invention may comprise 1 to 5% by weight of buffer, based on the total amount of the formulation prepared, to regulate the pH, the amount and type of the buffer used depending on the chemical properties of the active compound or the active compounds. Examples of buffers are alkali metal salts of weak inorganic or organic acids, such as, for example, phosphoric acid, boronic acid, acetic acid, propionic acid, citric acid, fumaric acid, tartaric acid, oxalic acid and succinic acid.

Substances which are suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, strongly polar solvents, for example dimethyl sulfoxide, N-methylpyrrolidone and water.

Powders, materials for spreading and dusts can be prepared by mixing or concomitantly grinding the active substances with a solid carrier.

Granules, for example coated granules, impregnated granules and homogeneous granules, can be prepared by binding the active ingredients to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.01 to 95% by weight, preferably from 0.1 to 90% by weight, of the active ingredient. The active ingredients are employed in a purity of from 90% to 100%, preferably 95% to 100% (according to NMR spectrum).

For seed treatment purposes, respective formulations can be diluted 2-10 fold leading to concentrations in the ready to use preparations of 0,01 to 60% by weight active compound by weight, preferably 0,1 to 40% by weight.

The compound of formula I or formula II can be used as such, in the form of their formulations or the use forms prepared therefrom, for example in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; they are intended to ensure in each case the finest possible distribution of the active compounds according to the invention.

The following are examples of formulations:

1. Products for dilution with water. For seed treatment purposes, such products may be applied to the seed diluted or undiluted.

### A) Water-Soluble Concentrates (SL, LS)

10 parts by weight of the active compound is dissolved in 90 parts by weight of water or a water-soluble solvent. As an alternative, wetters or other auxiliaries are added. The active compound dissolves upon dilution with water, whereby a formulation with 10% (w/w) of active compound is obtained.

### B) Dispersible Concentrates (DC)

20 parts by weight of the active compound is dissolved in 70 parts by weight of cyclo-hexanone with addition of 10 parts by weight of a dispersant, for example polyvinylpyrrolidone. Dilution with water gives a dispersion, whereby a formulation with 20% (w/w) of active compounds is obtained.

### C) Emulsifiable Concentrates (EC)

15 parts by weight of the active compounds is dissolved in 7 parts by weight of xylene with addition of calcium dodecylbenzenesulfonate and castor oil ethoxylate (in each case 5 parts by weight). Dilution with water gives an emulsion, whereby a formulation with 15% (w/w) of active compounds is obtained.

### D) Emulsions (EW, EO, ES)

25 parts by weight of the active compound is dissolved in 35 parts by weight of xylene with addition of calcium dodecylbenzenesulfonate and castor oil ethoxylate (in each case 5 parts by weight). This mixture is introduced into 30 parts by weight of water by means of an emulsifier machine (e.g. Ultraturrax) and made into a homogeneous emulsion. Dilution with water gives an emulsion, whereby a formulation with 25% (w/w) of active compound is obtained.

### E) Suspensions (SC, OD, FS)

In an agitated ball mill, 20 parts by weight of the active compound is comminuted with addition of 10 parts by weight of dispersants, wetters and 70 parts by weight of water or of an organic solvent to give a fine active compound suspension. Dilution with water gives a stable suspension of the active compound, whereby a formulation with 20% (w/w) of active compound is obtained.

### F) Water-Dispersible Granules and Water-Soluble Granules (WG, SG)

50 parts by weight of the active compound is ground finely with addition of 50 parts by weight of dispersants and wetters and made as water-dispersible or water-soluble granules by means of technical appliances (for example extrusion, spray tower, fluidized bed). Dilution with water gives a stable dispersion or solution of the active compound, whereby a formulation with 50% (w/w) of active compound is obtained.

### G) Water-Dispersible Powders and Water-Soluble Powders (WP, SP, SS, WS)

75 parts by weight of the active compound are ground in a rotor-stator mill with addition of 25 parts by weight of dispersants, wetters and silica gel. Dilution with water gives a stable dispersion or solution of the active compound, whereby a formulation with 75% (w/w) of active compound is obtained.

### H) Gel-Formulation (GF)

In an agitated ball mill, 20 parts by weight of the active compound is comminuted with addition of 10 parts by weight of dispersants, 1 part by weight of a gelling agent wetters and 70 parts by weight of water or of an organic solvent to give a fine active compound suspension. Dilution with water gives a stable suspension of the active compound, whereby a formulation with 20% (w/w) of active compound is obtained.

2. Products to be applied undiluted for foliar applications. For seed treatment purposes, such products may be applied to the seed diluted or undiluted.

### I) Dustable Powders (DP, DS)

5 parts by weight of the active compound are ground finely and mixed intimately with 95 parts by weight of finely divided kaolin. This gives a dustable product having 5% (w/w) of active compound.

### J) Granules (GR, FG, GG, MG)

0.5 part by weight of the active compound is ground finely and associated with 95.5 parts by weight of carriers, whereby a formulation with 0.5% (w/w) of active compound is obtained. Current methods are extrusion, spray-drying or the fluidized bed. This gives granules to be applied undiluted for foliar use.

### K) ULV Solutions (UL)

10 parts by weight of the active compound is dissolved in 90 parts by weight of an organic solvent, for example xylene. This gives a product having 10% (w/w) of active compound, which is applied undiluted for foliar use.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. Alternatively, it is possible to prepare concentrates composed of active substance, wetter, tackifier, dispersant or emulsifier and, if appropriate, solvent or oil, and such concentrates are suitable for dilution with water.

The active ingredient concentrations in the ready-to-use products can be varied within relatively wide ranges. In general, they are from 0.0001 to 10%, preferably from 0.01 to 1%.

The active ingredients may also be used successfully in the ultra-low-volume process (ULV), it being possible to apply formulations comprising over 95% by weight of active ingredient, or even to apply the active ingredient without additives.

In the method of this invention compounds of formula I or II may be applied with other active ingredients, for example with other pesticides, insecticides, herbicides, fertilizers such as ammonium nitrate, urea, potash, and superphosphate, phytotoxicants and plant growth regulators, safeners and nematicides. These additional ingredients may be used sequentially or in combination with the above-described compositions, if appropriate also added only immediately prior to use (tank mix). For example, the plant(s) may be sprayed with a composition of this invention either before or after being treated with other active ingredients.

The following list M of pesticides together with which the compounds according to the invention can be used and with which potential synergistic effects might be produced, is intended to illustrate the possible combinations, but not to impose any limitation:

M.1. Organo(thio)phosphate compounds: acephate, azamethiphos, azinphos-ethyl, azinphos-methyl, chlorethoxyfos, chlorfenvinphos, chlormephos, chlorpyrifos, chlorpyrifos-methyl, coumaphos, cyanophos, demeton-S-methyl, diazinon, dichlorvos/DDVP, dicrotophos, dimethoate, dimethylvinphos, disulfoton, EPN, ethion, ethoprophos, famphur, fenamiphos, fenitrothion, fenthion, flupyrazophos, fosthiazate, heptenophos, isoxathion, malathion, mecarbam, methamidophos, methidathion, mevinphos, monocrotophos, naled, omethoate, oxydemeton-methyl, parathion, parathion-methyl, phenthoate, phorate, phosalone, phosmet, phosphamidon, phoxim, pirimiphos-methyl, profenofos, propetamphos, prothiofos, pyraclofos, pyridaphenthion, quinalphos, sulfotep, tebupirimfos, temephos, terbufos, tetrachlorvinphos, thiometon, triazophos, trichlorfon, vamidothion;

M.2. Carbamate compounds: aldicarb, alanycarb, bendiocarb, benfuracarb, butocarboxim, butoxycarboxim, carbaryl, carbofuran, carbosulfan, ethiofencarb, fenobucarb, formetanate, furathiocarb, isoprocarb, methiocarb, methomyl, metolcarb, oxamyl, pirimicarb, propoxur, thiodicarb, thiofanox, trimethacarb, XMC, xylylcarb, triazamate;

M.3. Pyrethroid compounds: acrinathrin, allethrin, d-cis-trans allethrin, d-trans allethrin, bifenthrin, bioallethrin, bioallethrin S-cylclopentenyl, bioresmethrin, cycloprothrin, cyfluthrin, beta-cyfluthrin, cyhalothrin, lambda-cyhalothrin, gamma-cyhalothrin, cypermethrin, alpha-cypermethrin, beta-cypermethrin, theta-cypermethrin, zeta-cypermethrin, cyphenothrin, deltamethrin, empenthrin, esfenvalerate, etofenprox, fenpropathrin, fenvalerate, flucythrinate, flumethrin, tau-fluvalinate, halfenprox, imiprothrin, metofluthrin, permethrin, phenothrin, prallethrin, profluthrin, pyrethrin (pyrethrum), resmethrin, silafluofen, tefluthrin, tetramethrin, tralomethrin, transfluthrin;

M.4. Juvenile hormone mimics: hydroprene, kinoprene, methoprene, fenoxycarb, pyriproxyfen;

M.5. Nicotinic receptor agonists/antagonists compounds: acetamiprid, bensultap, cartap hydrochloride, clothianidin, dinotefuran, imidacloprid, thiamethoxam, nitenpyram, nicotine, spinosad (allosteric agonist), spinetoram (allosteric agonist), thiacloprid, thiocyclam, thiosultap-sodium and AKD1022.

M.6. GABA gated chloride channel antagonist compounds: chlordane, endosulfan, gamma-HCH (lindane); ethiprole, fipronil, pyrafluprole, pyriprole

M.7. Chloride channel activators: abamectin, emamectin benzoate, milbemectin, lepimectin;

M.8. METI I compounds: fenazaquin, fenpyroximate, pyrimidifen, pyridaben, tebufenpyrad, tolfenpyrad, flufenerim, rotenone;

M.9. METI II and III compounds: acequinocyl, fluacyprim, hydramethylnon;

M.10. Uncouplers of oxidative phosphorylation: chlorfenapyr, DNOC;

M.11. Inhibitors of oxidative phosphorylation: azocyclotin, cyhexatin, diafenthiuron, fenbutatin oxide, propargite, tetradifon;

M.12. Moulting disruptors: cyromazine, chromafenozide, halofenozide, methoxyfenozide, tebufenozide;

M.13. Synergists: piperonyl butoxide, tribufos;

M.14. Sodium channel blocker compounds: indoxacarb, metaflumizone;

M.15. Fumigants: methyl bromide, chloropicrin sulfuryl fluoride;

M.16. Selective feeding blockers: crylotie, pymetrozine, flonicamid;

M.17. Mite growth inhibitors: clofentezine, hexythiazox, etoxazole;

M.18. Chitin synthesis inhibitors: buprofezin, bistrifluoron, chlorfluazuron, diflubenzuron, flucycloxuron, flufenoxuron, hexaflumuron, lufenuron, novaluron, noviflumuron, teflubenzuron, triflumuron;

M.19. Lipid biosynthesis inhibitors: spirodiclofen, spiromesifen, spirotetramat;

M.20. Octapaminergic agonsits: amitraz;

M.21. Ryanodine receptor modulators: flubendiamide and the phtalamid compound (R)—, (S)— 3-Chlor-N-1-{2-methyl-4[1,2,2,2-tetrafluor-1-(trifluormethyl)ethyl]phenyl}-N2-(1-methyl-2-methylsulfonylethyl)phthalamid (M21.1)

M.22. Isoxazoline compounds: 4-[5-(3,5-Dichloro-phenyl)-5-trifluoromethyl-4,5-dihydro-isoxazol-3-yl]-2-methyl-N-pyridin-2-ylmethyl-benzamide (M22.1), 4-[5-(3,5-Dichloro-phenyl)-5-trifluoromethyl-4,5-dihydro-isoxazol-3-yl]-2-methyl-N-(2,2,2-trifluoro-ethyl)-benzamide (M22.2), 4-[5-(3,5-Dichloro-phenyl)-5-trifluoromethyl-4,5-dihydro-isoxazol-3-yl]-2-methyl-N-[(2,2,2-trifluoro-ethylcarbamoyl)-methyl]-benzamide (M22.3), 4-[5-(3,5-Dichloro-phenyl)-5-trifluoromethyl-4,5-dihydro-isoxazol-3-yl]-naphthalene-1-carboxylic acid [(2,2,2-trifluoro-ethylcarbamoyl)-methyl]-amide (M22.4) and 4-[5-(3,5-Dichlorophenyl)-5-trifluoromethyl-4,5-dihydro-isoxazol-3-yl]-N-[(methoxyimino)methyl]-2-methylbenzamide (M22.5);

M.23. Anthranilamide compounds: chloranthraniliprole, cyantraniliprole, 5-Bromo-2-(3-chloro-pyridin-2-yl)-2H-pyrazole-3-carboxylic acid [4-cyano-2-(1-cyclopropyl-ethylcarbamoyl)-6-methyl-phenyl]-amide (M23.1), 5-Bromo-2-(3-chloro-pyridin-2-yl)-2H-pyrazole-3-carboxylic acid [2-chloro-4-cyano-6-(1-cyclopropyl-ethylcarbamoyl)-phenyl]-amide (M23.2), 5-Bromo-2-(3-chloro-pyridin-2-yl)-2H-pyrazole-3-carboxylic acid [2-bromo-4-cyano-6-(1-cyclopropyl-ethylcarbamoyl)-phenyl]-amide(M23.3), 5-Bromo-2-(3-chloro-pyridin-2-yl)-2H-pyrazole-3-carboxylic acid [2-bromo-4-chloro-6-(1-cyclopropyl-ethylcarbamoyl)-phenyl]-amide(M23.4), 5-Bromo-2-(3-chloro-pyridin-2-yl)-2H-pyrazole-3-carboxylic acid [2,4-dichloro-6-(1-cyclopropyl-ethylcarbamoyl)-phenyl]-amide (M23.5), 5-Bromo-2-(3-chloro-pyridin-2-yl)-2H-pyrazole-3-carboxylic acid [4-chloro-2-(1-cyclopropyl-ethylcarbamoyl)-6-methyl-phenyl]amide (M23.6), N′-(2-{[5-Bromo-2-(3-chloro-pyridin-2-yl)-2H-pyrazole-3-carbonyl]-amino}-5-chloro-3-methyl-benzoyl)-hydrazinecarboxylic acid methyl ester (M23.7), N′-(2-{[5-Bromo-2-(3-chloro-pyridin-2-yl)-2H-pyrazole-3-carbonyl]-amino}-5-chloro-3-methyl-benzoyl)-N′-methyl-hydrazinecarboxylic acid methyl ester (M23.8), N′-(2-{[5-Bromo-2-(3-chloro-pyridin-2-yl)-2H-pyrazole-3-carbonyl]-amino}-5-chloro-3-methyl-benzoyl)-N,N′-dimethyl-hydrazinecarboxylic acid methyl ester (M23.9), N′-(3,5-Dibromo-2-{[5-bromo-2-(3-chloro-pyridin-2-yl)-2H-pyrazole-3-carbonyl]-amino}-benzoyl)-hydrazinecarboxylic acid methyl ester (M23.10), N′-(3,5-Dibromo-2-{[5-bromo-2-(3-chloro-pyridin-2-yl)-2H-pyrazole-3-carbonyl]-amino}-benzoyl)-N′-methyl-hydrazinecarboxylic acid methyl ester (M23.11) and N′-(3,5-Dibromo-2-{[5-bromo-2-(3-chloro-pyridin-2-yl)-2H-pyrazole-3-carbonyl]-amino}-benzoyl)-N,N′-dimethyl-hydrazinecarboxylic acid methyl ester (M23.12);

M.24. Malononitrile compounds: 2-(2,2,3,3,4,4,5,5-octafluoropentyl)-2-(3,3,3-trifluoro-propyl)malononitrile (CF₂H—CF₂—CF₂—CF₂—CH₂—C(CN)₂—CH₂—CH₂—CF₃) (M24.1) and 2-(2,2,3,3,4,4,5,5-octafluoropentyl)-2-(3,3,4,4,4-pentafluorobutyl)-malonodinitrile (CF₂H—CF₂—CF₂—CF₂—CH₂—C(CN)₂—CH₂—CH₂—CF₂—CF₃) (M24.2);

M.25. Microbial disruptors: Bacillus thuringiensis subsp. Israelensi, Bacillus sphaericus, Bacillus thuringiensis subsp. Aizawai, Bacillus thuringiensis subsp. Kurstaki, Bacillus thuringiensis subsp. Tenebrionis;

M.26. Aminofuranone compounds: 4-{[(6-Bromopyrid-3-yl)methyl](2-fluoroethyl)amino}furan-2(5H)-on (M26.1), 4-{[(6-Fluoropyrid-3-yl)methyl](2,2-difluoroethyl)amino}furan-2(5H)-on (M26.2), 4-{[(2-Chloro 1,3-thiazolo-5-yl)methyl](2-fluoroethyl)amino}furan-2(5H)-on (M26.3), 4-{[(6-Chloropyrid-3-yl)methyl](2-fluoroethyl)amino}furan-2(5H)-on (M26.4), 4-{[(6-Chloropyrid-3-yl)methyl](2,2-difluoroethyl)amino}furan-2(5H)-on (M26.5), 4-{[(6-Chloro-5-fluoropyrid-3-yl)methyl](methyl)amino}furan-2(5H)-on (M26.6), 4-{[(5,6-Dichloropyrid-3-yl)methyl](2-fluoroethyl)amino}furan-2(5H)-on (M26.7), 4-{[(6-Chloro-5-fluoropyrid-3-yl)methyl](cyclopropyl)amino}furan-2(5H)-on (M26.8), 4-{[(6-Chloropyrid-3-yl)methyl](cyclopropyl)amino}furan-2(5H)-on (M26.9) and 4-{[(6-Chloropyrid-3-yl)methyl](methyl)amino}furan-2(5H)-on (M26.10);

M.27. Various compounds: aluminium phosphide, amidoflumet, benclothiaz, benzoximate, bifenazate, borax, bromopropylate, cyanide, cyenopyrafen, cyflumetofen, chinomethionate, dicofol, fluoroacetate, phosphine, pyridalyl, pyrifluquinazon, sulfur, organic sulfur compounds, tartar emetic, sulfoxaflor, N—R′-2,2-dihalo-1-R″cyclo-propanecarboxamide-2-(2,6-dichloro-α, α, α-trifluoro-p-tolyl)hydrazone or N—R′-2,2-di(R′″)propionamide-2-(2,6-dichloro-α, α,α-trifluoro-p-tolyl)-hydrazone, wherein R′ is methyl or ethyl, halo is chloro or bromo, R″ is hydrogen or methyl and R′″ is methyl or ethyl, 4-But-2-ynyloxy-6-(3,5-dimethyl-piperidin-1-yl)-2-fluoro-pyrimidine (M27.1), Cyclopropaneacetic acid, 1,1′-[(3S,4R,4aR,6S,6aS,12R,12aS,12b5)-4-[[(2-cyclopropylacetyl)oxy]methyl]-1,3,4,4a,5,6,6a,12,12a,12b-decahydro-12-hydroxy-4,6a,12b-trimethyl-11-oxo-9-(3-pyridinyl)-2H,11H-naphtho[2,1-b]pyrano[3,4-e]pyran-3,6-diyl]ester(M27.2) and 8-(2-Cyclopropylmethoxy-4-trifluoromethyl-phenoxy)-3-(6-trifluoromethyl-pyridazin-3-yl)-3-aza-bicyclo[3.2.1]octane(M27.3).

The commercially available compounds of the group M may be found in The Pesticide Manual, 13th Edition, British Crop Protection Council (2003) among other publications.

Lepimectin is known from Agro Project, PJB Publications Ltd, November 2004. Benclothiaz and its preparation have been described in EP-A1454621. Methidathion and Paraoxon and their preparation have been described in Farm Chemicals Handbook, Volume 88, Meister Publishing Company, 2001. Metaflumizone and its preparation have been described in EP-A1462 456. Flupyrazofos has been described in Pesticide Science 54, 1988, p. 237-243 and in U.S. Pat. No. 4,822,779. Pyrafluprole and its preparation have been described in JP 2002193709 and in WO 01/00614. Pyriprole and its preparation have been described in WO 98/45274 and in U.S. Pat. No. 6,335,357. Amidoflumet and its preparation have been described in U.S. Pat. No. 6,221,890 and in JP 21010907. Flufenerim and its preparation have been described in WO 03/007717 and in WO 03/007718. AKD 1022 and its preparation have been described in U.S. Pat. No. 6,300,348. Chloranthraniliprole has been described in WO 01/70671, WO 03/015519 and WO 05/118552. Cyantraniliprole has been described in WO 01/70671, WO 04/067528 and WO 05/118552. The anthranilamides M23.1 to M23.6 have been described in WO 2008/72743 and WO 200872783, those M23.7 to M23.12 in WO2007/043677. The phthalamide M 21.1 is known from WO 2007/101540. Cyflumetofen and its preparation have been described in WO 04/080180. The aminoquinazolinone compound pyrifluquinazon has been described in EP A 109 7932. Sulfoximine sulfoxaflor has been described in WO 2006/060029 and WO 2007/149134. The alkynylether compound M27.1 is described e.g. in JP 2006131529. Organic sulfur compounds have been described in WO 2007060839. The isoxazoline compounds M 22.1 to M 22.5 have been described in e.g. WO2005/085216, WO 2007/079162 and WO 2007/026965. The aminofuranone compounds M 26.1 to M 26.10 have been described e.g. in WO 2007/115644. The pyripyropene derivative M 27.2 has been described in WO 2008/66153 and WO 2008/108491. The pyridazin compound M 27.3 has been described in JP 2008/115155. Malononitrile compounds as those (M24.1) and (M24.2) have been described in WO 02/089579, WO 02/090320, WO 02/090321, WO 04/006677, WO 05/068423, WO 05/068432 and WO 05/063694.

Fungicidal mixing partners are those selected from the group consisting of acylalanines such as benalaxyl, metalaxyl, ofurace, oxadixyl, amine derivatives such as aldimorph, dodine, dodemorph, fenpropimorph, fenpropidin, guazatine, iminoctadine, spiroxamin, tridemorph, anilinopyrimidines such as pyrimethanil, mepanipyrim or cyrodinyl, antibiotics such as cycloheximid, griseofulvin, kasugamycin, natamycin, polyoxin or streptomycin,

azoles such as bitertanol, bromoconazole, cyproconazole, difenoconazole, diniconazole, epoxiconazole, fenbuconazole, fluquiconazole, flusilazole, hexaconazole, imazalil, metconazole, myclobutanil, penconazole, propiconazole, prochloraz, prothioconazole, tebuconazole, triadimefon, triadimenol, triflumizol, triticonazole, flutriafol, dicarboximides such as iprodion, myclozolin, procymidon, vinclozolin, dithiocarbamates such as ferbam, nabam, maneb, mancozeb, metam, metiram, propineb, polycarbamate, thiram, ziram, zineb,
heterocyclic compounds such as anilazine, benomyl, boscalid, carbendazim, carboxin, oxycarboxin, cyazofamid, dazomet, dithianon, famoxadon, fenamidon, fenarimol, fuberidazole, flutolanil, furametpyr, isoprothiolane, mepronil, nuarimol, probenazole, proquinazid, pyrifenox, pyroquilon, quinoxyfen, silthiofam, thiabendazole, thifluzamid, thiophanate-methyl, tiadinil, tricyclazole, triforine,
copper fungicides such as Bordeaux mixture, copper acetate, copper oxychloride, basic copper sulfate,
nitrophenyl derivatives such as binapacryl, dinocap, dinobuton, nitrophthalisopropyl, phenylpyrroles such as fenpiclonil or fludioxonil,
sulfur,
other fungicides such as acibenzolar-S-methyl, benthiavalicarb, carpropamid, chlorothalonil, cyflufenamid, cymoxanil, diclomezin, diclocymet, diethofencarb, edifenphos, ethaboxam, fenhexamid, fentin-acetate, fenoxanil, ferimzone, fluazinam, fosetyl, fosetyl-aluminum, iprovalicarb, hexachlorobenzene, metrafenon, pencycuron, propamocarb, phthalide, toloclofos-methyl, quintozene, zoxamid,
strobilurins such as azoxystrobin, dimoxystrobin, fluoxastrobin, kresoxim-methyl, metominostrobin, orysastrobin, picoxystrobin or trifloxystrobin,
sulfenic acid derivatives such as captafol, captan, dichlofluanid, folpet, tolylfluanid, cinnemamides and analogs such as dimethomorph, flumetover or flumorph.

### APPLICATIONS

Due to their excellent activity, the compounds of formulae I and II may be used for controlling animal pests.

Accordingly, the present invention also provides a method for controlling animal pests which method comprises treating the pests, their food supply, their habitat or their breeding ground or a cultivated plant, plant propagation materials (such as seed), soil, area, material or environment in which the pests are growing or may grow, or the materials, cultivated plants, plant propagation materials (such as seed), soils, surfaces or spaces to be protected from pest attack or infestation with a pesticidally effective amount of a compound of formula I or II or a salt thereof or a composition as defined above.

Preferably, the method of the invention serves for protecting plant propagation material (such as seed) and the plant which grows therefrom from animal pest attack or infestation and comprises treating the plant propagation material (such as seed) with a pesticidally effective amount of a compound of the formula I or II or an agriculturally acceptable salt thereof as defined above or with a pesticidally effective amount of an agricultural composition as defined above and below. The method of the invention is not limited to the protection of the “substrate” (plant, plant propagation materials, soil material etc.) which has been treated according to the invention, but also has a preventive effect, thus, for example, according protection to a plant which grows from a treated plant propagation materials (such as seed), the plant itself not having been treated.

In the sense of the present invention, “animal pests” are preferably selected from arthropods and nematodes, more preferably from harmful insects, arachnids and nematodes, and even more preferably from insects, acarids and nematodes.

The invention further provides an agricultural composition for combating such animal pests, which comprises such an amount of at least one compound of formula I or II or at least one agriculturally useful salt thereof and at least one inert liquid and/or solid agronomically acceptable carrier that has a pesticidal action and, if desired, at least one surfactant.

Such a composition may contain a single active compound of formula I or II or a salt thereof or a mixture of several active compounds of formulae I or II or their salts according to the present invention. The composition according to the present invention may comprise an individual isomer or mixtures of isomers as well as individual tautomers or mixtures of tautomers.

The animal pest, i.e. the insects, arachnids and nematodes, the plant, soil or water in which the plant is growing can be contacted with the present compounds of formula I or composition(s) containing them by any application method known in the art. As such, “contacting” includes both direct contact (applying the compounds/compositions directly on the animal pest or plant—typically to the foliage, stem or roots of the plant) and indirect contact (applying the compounds/compositions to the locus of the animal pest or plant).

The compounds of formula I or the pesticidal compositions comprising them may be used to protect growing plants and crops from attack or infestation by animal pests, especially insects, acaridae or arachnids by contacting the plant/crop with a pesticidally effective amount of compounds of formula I. The term “crop” refers both to growing and harvested crops.

The compounds of the present invention and the compositions comprising them are particularly important in the control of a multitude of insects on various cultivated plants, such as cereal, root crops, oil crops, vegetables, spices, ornamentals, for example seed of durum and other wheat, barley, oats, rye, maize (fodder maize and sugar maize/sweet and field corn), soybeans, oil crops, crucifers, cotton, sunflowers, bananas, rice, oilseed rape, turnip rape, sugarbeet, fodder beet, eggplants, potatoes, grass, lawn, turf, fodder grass, tomatoes, leeks, pumpkin/squash, cabbage, iceberg lettuce, pepper, cucumbers, melons, Brassica species, melons, beans, peas, garlic, onions, carrots, tuberous plants such as potatoes, sugar cane, tobacco, grapes, petunias, geranium/pelargoniums, pansies and impatiens.

The compounds of the present invention are employed as such or in form of compositions by treating the insects or the plants, plant propagation materials, such as seeds, soil, surfaces, materials or rooms to be protected from insecticidal attack with a insecticidally effective amount of the active compounds. The application can be carried out both before and after the infection of the plants, plant propagation materials, such as seeds, soil, surfaces, materials or rooms by the insects.

The present invention also includes a method of combating animal pests which comprises contacting the animal pests, their habit, breeding ground, food supply, cultivated plants, seed, soil, area, material or environment in which the animal pests are growing or may grow, or the materials, plants, seeds, soils, surfaces or spaces to be protected from animal attack or infestation with a pesticidally effective amount of a mixture of at least one active compound of formula I and II.

Moreover, animal pests may be controlled by contacting the target pest, its food supply, habitat, breeding ground or its locus with a pesticidally effective amount of compounds of formula I or II. As such, the application may be carried out before or after the infection of the locus, growing crops, or harvested crops by the pest.

The compounds of the invention can also be applied preventively to places at which occurrence of the pests is expected.

The compounds of formula I and II may be also used to protect growing plants from attack or infestation by pests by contacting the plant with a pesticidally effective amount of compounds of formula I. As such, “contacting” includes both direct contact (applying the compounds/compositions directly on the pest and/or plant—typically to the foliage, stem or roots of the plant) and indirect contact (applying the compounds/compositions to the locus of the pest and/or plant).

“Locus” means a habitat, breeding ground, plant, seed, soil, area, material or environment in which a pest or parasite is growing or may grow.

The term “plant propagation material” is to be understood to denote all the generative parts of the plant such as seeds and vegetative plant material such as cuttings and tubers (e.g. potatoes), which can be used for the multiplication of the plant. This includes seeds, roots, fruits, tubers, bulbs, rhizomes, shoots, sprouts and other parts of plants. Seedlings and young plants, which are to be transplanted after germination or after emergence from soil, may also be included. These plant propagation materials may be treated prophylactically with a plant protection compound either at or before planting or transplanting.

The term “cultivated plants” is to be understood as including plants which have been modified by breeding, mutagenesis or genetic engineering. Genetically modified plants are plants, which genetic material has been so modified by the use of recombinant DNA techniques that under natural circumstances cannot readily be obtained by cross breeding, mutations or natural recombination. Typically, one or more genes have been integrated into the genetic material of a genetically modified plant in order to improve certain properties of the plant. Such genetic modifications also include but are not limited to targeted post-transitional modification of protein(s) (oligo- or polypeptides) poly for example by glycosylation or polymer additions such as prenylated, acetylated or farnesylated moieties or PEG moieties(e.g. as disclosed in Biotechnol Prog. 2001 Jul.-Aug.; 17(4):720-8., Protein Eng Des Sel. 2004 Jan.; 17(1):57-66, Nat. Protoc. 2007; 2(5):1225-35., Curr Opin Chem. Biol. 2006 Oct.; 10(5):487-91. Epub 2006 Aug. 28., Biomaterials. 2001 Mar.; 22(5):405-17, Bioconjug Chem. 2005 Jan.-Feb.; 16(1):113-21).

The term “cultivated plants” is to be understood also including plants that have been rendered tolerant to applications of specific classes of herbicides, such as hydroxy-phenylpyruvate dioxygenase (HPPD) inhibitors; acetolactate synthase (ALS) inhibitors, such as sulfonyl ureas (see e.g. U.S. Pat. No. 6,222,100, WO 01/82685, WO 00/26390, WO 97/41218, WO 98/02526, WO 98/02527, WO 04/106529, WO 05/20673, WO 03/14357, WO 03/13225, WO 03/14356, WO 04/16073) or imidazolinones (see e.g. U.S. Pat. No. 6,222,100, WO 01/82685, WO 00/26390, WO 97/41218, WO 98/02526, WO 98/02527, WO 04/106529, WO 05/20673, WO 03/14357, WO 03/13225, WO 03/14356, WO 04/16073); enolpyruvylshikimate-3-phosphate synthase (EPSPS) inhibitors, such as glyphosate (see e.g. WO 92/00377); glutamine synthetase (GS) inhibitors, such as glufosinate (see e.g. EP-A-0242236, EP-A-242246) or oxynil herbicides (see e.g. U.S. Pat. No. 5,559,024) as a result of conventional methods of breeding or genetic engineering. Several cultivated plants have been rendered tolerant to herbicides by conventional methods of breeding (mutagenesis), for example Clearfield® summer rape (Canola) being tolerant to imidazolinones, e.g. imazamox. Genetic engineering methods have been used to render cultivated plants, such as soybean, cotton, corn, beets and rape, tolerant to herbicides, such as glyphosate and glufosinate, some of which are commercially available under the trade names RoundupReady® (glyphosate) and LibertyLink® (glufosinate).

The term “cultivated plants” is to be understood also including plants that are by the use of recombinant DNA techniques capable to synthesize one or more insecticidal proteins, especially those known from the bacterial genus Bacillus, particularly from Bacillus thuringiensis, such as ä-endotoxins, e.g. CryIA(b), CryIA(c), CryIF, CryIF(a2), CryIIA(b), CryIIIA, CryIIIB(b1) or Cry9c; vegetative insecticidal proteins (VIP), e.g. VIP1, VIP2, VIP3 or VIP3A; insecticidal proteins of bacteria colonizing nematodes, for example Photorhabdus spp. or Xenorhabdus spp.; toxins produced by animals, such as scorpion toxins, arachnid toxins, wasp toxins, or other insect-specific neurotoxins; toxins produced by fungi, such Streptomycetes toxins, plant lectins, such as pea or barley lectins; agglutinins; proteinase inhibitors, such as trypsin inhibitors, serine protease inhibitors, patatin, cystatin or papain inhibitors; ribosome-inactivating proteins (RIP), such as ricin, maize-RIP, abrin, luffin, saporin or bryodin; steroid metabolism enzymes, such as 3-hydroxysteroid oxidase, ecdysteroid-IDP-glycosyl-transferase, cholesterol oxidases, ecdysone inhibitors or HMG-CoA-reductase; ion channel blockers, such as blockers of sodium or calcium channels; juvenile hormone esterase; diuretic hormone receptors (helicokinin receptors); stilben synthase, bibenzyl synthase, chitinases or glucanases. In the context of the present invention these insecticidal proteins or toxins are to be understood expressly also as pre-toxins, hybrid proteins, truncated or otherwise modified proteins. Hybrid proteins are characterized by a new combination of protein domains, (see, for example WO 02/015701). Further examples of such toxins or genetically-modified plants capable of synthesizing such toxins are dis-closed, for example, in EP-A 374 753, WO 93/007278, WO 95/34656, EP-A 427 529, EP-A 451 878, WO 03/018810 and WO 03/052073. The methods for producing such genetically modified plants are generally known to the person skilled in the art and are described, for example, in the publications mentioned above. These insecticidal proteins contained in the genetically modified plants impart to the plants producing these proteins protection from harmful pests from certain taxonomic groups of arthropods, particularly to beetles (Coleoptera), flies (Diptera), and butterflies and moths (Lepidoptera) and to plant parasitic nematodes (Nematoda).

The term “cultivated plants” is to be understood also including plants that are by the use of recombinant DNA techniques capable to synthesize one or more proteins to increase the resistance or tolerance of those plants to bacterial, viral or fungal pathogens. Examples of such proteins are the so-called “pathogenesis-related proteins” (PR proteins, see, for example EP-A 0 392 225), plant disease resistance genes (for example potato cultivars, which express resistance genes acting against Phytophthora infestans derived from the Mexican wild potato Solanum bulbocastanum) or T4-lyso-zym (e.g. potato cultivars capable of synthesizing these proteins with increased resistance against bacteria such as Erwinia amylvora). The methods for producing such genetically modified plants are generally known to the person skilled in the art and are described, for example, in the publications mentioned above.

The term “cultivated plants” is to be understood also including plants that are by the use of recombinant DNA techniques capable to synthesize one or more proteins to increase the productivity (e.g. bio mass production, grain yield, starch content, oil content or protein content), tolerance to drought, salinity or other growth-limiting environ-mental factors or tolerance to pests and fungal, bacterial or viral pathogens of those plants.

The term “cultivated plants” is to be understood also including plants that contain by the use of recombinant DNA techniques a modified amount of substances of content or new substances of content, specifically to improve human or animal nutrition, for example oil crops that produce health-promoting long-chain omega-3 fatty acids or unsaturated omega-9 fatty acids (e.g. Nexera® rape).

The term “cultivated plants” is to be understood also including plants that contain by the use of recombinant DNA techniques a modified amount of substances of content or new substances of content, specifically to improve raw material production, for example potatoes that produce increased amounts of amylopectin (e.g. Amflora® potato).

In general, “pesticidally effective amount” means the amount of active ingredient needed to achieve an observable effect on growth, including the effects of necrosis, death, retardation, prevention, and removal, destruction, or otherwise diminishing the occurrence and activity of the target organism. The pesticidally effective amount can vary for the various compounds/compositions used in the invention. A pesticidally effective amount of the compositions will also vary according to the prevailing conditions such as desired pesticidal effect and duration, weather, target species, locus, mode of application, and the like.

In the case of soil treatment or of application to the pests dwelling place or nest, the quantity of active ingredient ranges from 0.0001 to 500 g per 100 m², preferably from 0.001 to 20 g per 100 m².

Customary application rates in the protection of materials are, for example, from 0.01 g to 1000 g of active compound per m² treated material, desirably from 0.1 g to 50 g per m².

Insecticidal compositions for use in the impregnation of materials typically contain from 0.001 to 95 weight %, preferably from 0.1 to 45 weight %, and more preferably from 1 to 25 weight % of at least one repellent and/or insecticide.

For use in treating crop plants, the rate of application of the active ingredients of this invention may be in the range of 0.1 g to 4000 g per hectare, desirably from 25 g to 600 g per hectare, more desirably from 50 g to 500 g per hectare.

The compounds of formula I and II are effective through both contact (via soil, glass, wall, bed net, carpet, plant parts or animal parts), and ingestion (bait, or plant part).

The compounds of the invention may also be applied against non-crop insect pests, such as ants, termites, wasps, flies, mosquitoes, crickets, or cockroaches. For use against said non-crop pests, compounds of formula I are preferably used in a bait composition.

The bait can be a liquid, a solid or a semisolid preparation (e.g. a gel). Solid baits can be formed into various shapes and forms suitable to the respective application e.g. granules, blocks, sticks, disks. Liquid baits can be filled into various devices to ensure proper application, e.g. open containers, spray devices, droplet sources, or evaporation sources. Gels can be based on aqueous or oily matrices and can be formulated to particular necessities in terms of stickiness, moisture retention or aging characteristics.

The bait employed in the composition is a product, which is sufficiently attractive to incite insects such as ants, termites, wasps, flies, mosquitoes, crickets etc. or cockroaches to eat it. The attractiveness can be manipulated by using feeding stimulants or sex pheromones. Food stimulants are chosen, for example, but not exclusively, from animal and/or plant proteins (meat-, fish- or blood meal, insect parts, egg yolk), from fats and oils of animal and/or plant origin, or mono-, oligo- or polyorganosaccharides, especially from sucrose, lactose, fructose, dextrose, glucose, starch, pectin or even molasses or honey. Fresh or decaying parts of fruits, crops, plants, animals, insects or specific parts thereof can also serve as a feeding stimulant. Sex pheromones are known to be more insect specific. Specific pheromones are described in the literature and are known to those skilled in the art.

For use in bait compositions, the typical content of active ingredient is from 0.001 weight % to 15 weight %, desirably from 0.001 weight % to 5% weight % of active compound.

Formulations of compounds of formula I or II as aerosols (e.g. in spray cans), oil sprays or pump sprays are highly suitable for the non-professional user for controlling pests such as flies, fleas, ticks, mosquitoes or cockroaches. Aerosol recipes are preferably composed of the active compound, solvents such as lower alcohols (e.g. methanol, ethanol, propanol, butanol), ketones (e.g. acetone, methyl ethyl ketone), paraffin hydrocarbons (e.g. kerosenes) having boiling ranges of approximately 50 to 250° C., dimethylformamide, N-methylpyrrolidone, dimethyl sulfoxide, aromatic hydrocarbons such as toluene, xylene, water, furthermore auxiliaries such as emulsifiers such as sorbitol monooleate, oleyl ethoxylate having 3-7 mol of ethylene oxide, fatty alcohol ethoxylate, perfume oils such as ethereal oils, esters of medium fatty acids with lower alcohols, aromatic carbonyl compounds, if appropriate stabilizers such as sodium benzoate, amphoteric surfactants, lower epoxides, triethyl orthoformate and, if required, propellants such as propane, butane, nitrogen, compressed air, dimethyl ether, carbon dioxide, nitrous oxide, or mixtures of these gases.

The oil spray formulations differ from the aerosol recipes in that no propellants are used.

For use in spray compositions, the content of active ingredient is from 0.001 to 80 weights %, preferably from 0.01 to 50 weight % and most preferably from 0.01 to 15 weight %.

The compounds of formula I and II and their respective compositions can also be used in mosquito and fumigating coils, smoke cartridges, vaporizer plates or long-term vaporizers and also in moth papers, moth pads or other heat-independent vaporizer systems.

Methods to control infectious diseases transmitted by insects (e.g. malaria, dengue and yellow fever, lymphatic filariasis, and leishmaniasis) with compounds of formula I and its respective compositions also comprise treating surfaces of huts and houses, air spraying and impregnation of curtains, tents, clothing items, bed nets, tsetse-fly trap or the like. Insecticidal compositions for application to fibers, fabric, knitgoods, nonwovens, netting material or foils and tarpaulins preferably comprise a mixture including the insecticide, optionally a repellent and at least one binder. Suitable repellents for example are N,N-Diethyl-meta-toluamide (DEET), N,N-diethylphenylacetamide (DEPA), 1-(3-cyclohexan-1-yl-carbonyl)-2-methylpiperine, (2-hydroxymethylcyclohexyl)acetic acid lactone, 2-ethyl-1,3-hexandiol, indalone, Methylneodecanamide (MNDA), a pyrethroid not used for insect control such as {(+/−)-3-allyl-2-methyl-4-oxocyclopent-2-(+)-enyl-(+)-trans-chrysantemate (Esbiothrin), a repellent derived from or identical with plant extracts like limonene, eugenol, (+)-Eucamalol (1), (−)-1-epi-eucamalol or crude plant extracts from plants like Eucalyptus maculate, Vitex rotundifolia, Cymbopogan martinii, Cymbopogan citratus (lemon grass), Cymopogan nartdus (citronella). Suitable binders are selected for example from polymers and copolymers of vinyl esters of aliphatic acids (such as such as vinyl acetate and vinyl versatate), acrylic and methacrylic esters of alcohols, such as butyl acrylate, 2-ethylhexylacrylate, and methyl acrylate, mono- and di-ethylenically unsaturated hydrocarbons, such as styrene, and aliphatic diens, such as butadiene.

The impregnation of curtains and bednets is done in general by dipping the textile material into emulsions or dispersions of the insecticide or spraying them onto the nets.

The compounds of formula I and II and their compositions can be used for protecting wooden materials such as trees, board fences, sleepers, etc. and buildings such as houses, outhouses, factories, but also construction materials, furniture, leathers, fibers, vinyl articles, electric wires and cables etc. from ants and/or termites, and for controlling ants and termites from doing harm to crops or human being (e.g. when the pests invade into houses and public facilities). The compounds of formula I are applied not only to the surrounding soil surface or into the under-floor soil in order to protect wooden materials but it can also be applied to lumbered articles such as surfaces of the under-floor concrete, alcove posts, beams, plywoods, furniture, etc., wooden articles such as particle boards, half boards, etc. and vinyl articles such as coated electric wires, vinyl sheets, heat insulating material such as styrene foams, etc. In case of application against ants doing harm to crops or human beings, the ant controller of the present invention is applied to the crops or the surrounding soil, or is directly applied to the nest of ants or the like.

### Seed Treatment

The compounds of formula I and II are also suitable for the treatment of seeds in order to protect the seed from insect pest, in particular from soil-living insect pests and the resulting plant's roots and shoots against soil pests and foliar insects.

The compounds of formula I and II are particularly useful for the protection of the seed from soil pests and the resulting plant's roots and shoots against soil pests and foliar insects. The protection of the resulting plant's roots and shoots is preferred. More preferred is the protection of resulting plant's shoots from piercing and sucking insects, wherein the protection from aphids is most preferred.

The present invention therefore comprises a method for the protection of seeds from insects, in particular from soil insects and of the seedlings' roots and shoots from insects, in particular from soil and foliar insects, said method comprising contacting the seeds before sowing and/or after pregermination with a compound of the general formula I or II or a salt thereof. Particularly preferred is a method, wherein the plant's roots and shoots are protected, more preferably a method, wherein the plants shoots are protected form piercing and sucking insects, most preferably a method, wherein the plants shoots are protected from aphids.

The term seed embraces seeds and plant propagules of all kinds including but not limited to true seeds, seed pieces, suckers, corms, bulbs, fruit, tubers, grains, cuttings, cut shoots and the like and means in a preferred embodiment true seeds.

The term seed treatment comprises all suitable seed treatment techniques known in the art, such as seed dressing, seed coating, seed dusting, seed soaking and seed pelleting.

The present invention also comprises seeds coated with or containing the active compound.

The term “coated with and/or containing” generally signifies that the active ingredient is for the most part on the surface of the propagation product at the time of application, although a greater or lesser part of the ingredient may penetrate into the propagation product, depending on the method of application. When the said propagation product is (re)planted, it may absorb the active ingredient.

Suitable seed is seed of cereals, root crops, oil crops, vegetables, spices, ornamentals, for example seed of durum and other wheat, barley, oats, rye, maize (fodder maize and sugar maize/sweet and field corn), soybeans, oil crops, crucifers, cotton, sunflowers, bananas, rice, oilseed rape, turnip rape, sugarbeet, fodder beet, eggplants, potatoes, grass, lawn, turf, fodder grass, tomatoes, leeks, pumpkin/squash, cabbage, iceberg lettuce, pepper, cucumbers, melons, Brassica species, melons, beans, peas, garlic, onions, carrots, tuberous plants such as potatoes, sugar cane, tobacco, grapes, petunias, geranium/pelargoniums, pansies and impatiens.

In addition, the active compound may also be used for the treatment seeds from plants, which tolerate the action of herbicides or fungicides or insecticides owing to breeding, including genetic engineering methods.

For example, the active compound can be employed in treatment of seeds from plants, which are resistant to herbicides from the group consisting of the sulfonylureas, imidazolinones, glufosinate-ammonium or glyphosate-isopropylammonium and analogous active substances (see for example, EP-A-0242236, EP-A-242246) (WO 92/00377) (EP-A-0257993, U.S. Pat. No. 5,013,659) or in transgenic crop plants, for example cotton, with the capability of producing Bacillus thuringiensis toxins (Bt toxins) which make the plants resistant to certain pests (EP-A-0142924, EP-A-0193259),

Furthermore, the active compound can be used also for the treatment of seeds from plants, which have modified characteristics in comparison with existing plants consist, which can be generated for example by traditional breeding methods and/or the generation of mutants, or by recombinant procedures). For example, a number of cases have been described of recombinant modifications of crop plants for the purpose of modifying the starch synthesized in the plants (e.g. WO 92/11376, WO 92/14827, WO 91/19806) or of transgenic crop plants having a modified fatty acid composition (WO 91/13972).

The seed treatment application of the active compound is carried out by spraying or by dusting the seeds before sowing of the plants and before emergence of the plants.

Compositions which are especially useful for seed treatment are e.g.:

A Soluble concentrates (SL, LS)

### D Emulsions (EW, EO, ES)

### E Suspensions (SC, OD, FS)

F Water-dispersible granules and water-soluble granules (WG, SG)
G Water-dispersible powders and water-soluble powders (WP, SP, WS)

### H Gel-Formulations (GF)

I Dustable powders (DP, DS)

Conventional seed treatment formulations include for example flowable concentrates FS, solutions LS, powders for dry treatment DS, water dispersible powders for slurry treatment WS, water-soluble powders SS and emulsion ES and EC and gel formulation GF. These formulations can be applied to the seed diluted or undiluted. Application to the seeds is carried out before sowing, either directly on the seeds or after having pregerminated the latter

In a preferred embodiment a FS formulation is used for seed treatment. Typically, a FS formulation may comprise 1-800 g/l of active ingredient, 1-200 g/l Surfactant, 0 to 200 g/l antifreezing agent, 0 to 400 g/l of binder, 0 to 200 g/l of a pigment and up to 1 liter of a solvent, preferably water.

Especially preferred FS formulations of compounds of formula I for seed treatment usually comprise from 0.1 to 80% by weight (1 to 800 g/l) of the active ingredient, from 0.1 to 20% by weight (1 to 200 g/l) of at least one surfactant, e.g. 0.05 to 5% by weight of a wetter and from 0.5 to 15% by weight of a dispersing agent, up to 20% by weight, e.g. from 5 to 20% of an anti-freeze agent, from 0 to 15% by weight, e.g. 1 to 15% by weight of a pigment and/or a dye, from 0 to 40% by weight, e.g. 1 to 40% by weight of a binder (sticker/adhesion agent), optionally up to 5% by weight, e.g. from 0.1 to 5% by weight of a thickener, optionally from 0.1 to 2% of an anti-foam agent, and optionally a preservative such as a biocide, antioxidant or the like, e.g. in an amount from 0.01 to 1% by weight and a filler/vehicle up to 100% by weight.

Seed Treatment formulations may additionally also comprise binders and optionally colorants.

Binders can be added to improve the adhesion of the active materials on the seeds after treatment. Suitable binders are homo- and copolymers from alkylene oxides like ethylene oxide or propylene oxide, polyvinylacetate, polyvinylalcohols, polyvinylpyrrolidones, and copolymers thereof, ethylene-vinyl acetate copolymers, acrylic homo- and copolymers, polyethyleneamines, polyethyleneamides and polyethyleneimines, poly-saccharides like celluloses, tylose and starch, polyolefin homo- and copolymers like olefin/maleic anhydride copolymers, polyurethanes, polyesters, polystyrene homo and copolymers

Optionally, also colorants can be included in the formulation. Suitable colorants or dyes for seed treatment formulations are Rhodamine B, C.I. Pigment Red 112, C.I. Solvent Red 1, pigment blue 15:4, pigment blue 15:3, pigment blue 15:2, pigment blue 15:1, pigment blue 80, pigment yellow 1, pigment yellow 13, pigment red 112, pigment red 48:2, pigment red 48:1, pigment red 57:1, pigment red 53:1, pigment orange 43, pigment orange 34, pigment orange 5, pigment green 36, pigment green 7, pigment white 6, pigment brown 25, basic violet 10, basic violet 49, acid red 51, acid red 52, acid red 14, acid blue 9, acid yellow 23, basic red 10, basic red 108.

Examples of a gelling agent is carrageen (Satiagel®)

In the treatment of seed, the application rates of the compounds I are generally from 0.1 g to 10 kg per 100 kg of seed, preferably from 1 g to 5 kg per 100 kg of seed, more preferably from 1 g to 1000 g per 100 kg of seed and in particular from 1 g to 200 g per 100 kg of seed.

The invention therefore also relates to seed comprising a compound of the formula I, or an agriculturally useful salt of I, as defined herein. The amount of the compound I or the agriculturally useful salt thereof will in general vary from 0.1 g to 10 kg per 100 kg of seed, preferably from 1 g to 5 kg per 100 kg of seed, in particular from 1 g to 1000 g per 100 kg of seed. For specific crops such as lettuce the rate can be higher.

### Animal Health

The compounds of formula I or II or the enantiomers or veterinarily acceptable salts thereof are in particular also suitable for being used for combating parasites in and on animals.

An object of the present invention is therefore also to provide new methods to control parasites in and on animals. Another object of the invention is to provide safer pesticides for animals. Another object of the invention is further to provide pesticides for animals that may be used in lower doses than existing pesticides. And another object of the invention is to provide pesticides for animals, which provide a long residual control of the parasites.

The invention also relates to compositions containing a parasiticidally effective amount of compounds of formula I or the enantiomers or veterinarily acceptable salts thereof and an acceptable carrier, for combating parasites in and on animals.

The present invention also provides a method for treating, controlling, preventing and protecting animals against infestation and infection by parasites, which comprises orally, topically or parenterally administering or applying to the animals a parasiticidally effective amount of a compound of formula I or II or the enantiomers or veterinarily acceptable salts thereof or a composition comprising it.

The invention also provides a process for the preparation of a composition for treating, controlling, preventing or protecting animals against infestation or infection by parasites which comprises a parasiticidally effective amount of a compound of formula I or II or the enantiomers or veterinarily acceptable salts thereof or a composition comprising it.

Activity of compounds against agricultural pests does not suggest their suitability for control of endo- and ectoparasites in and on animals which requires, for example, low, non-emetic dosages in the case of oral application, metabolic compatibility with the animal, low toxicity, and a safe handling.

Surprisingly it has now been found that compounds of formula I and II are suitable for combating endo- and ectoparasites in and on animals.

Compounds of formula I or II or the enantiomers or veterinarily acceptable salts thereof and compositions comprising them are preferably used for controlling and preventing infestations and infections animals including warm-blooded animals (including humans) and fish. They are for example suitable for controlling and preventing infestations and infections in mammals such as cattle, sheep, swine, camels, deer, horses, pigs, poultry, rabbits, goats, dogs and cats, water buffalo, donkeys, fallow deer and reindeer, and also in fur-bearing animals such as mink, chinchilla and raccoon, birds such as hens, geese, turkeys and ducks and fish such as fresh- and salt-water fish such as trout, carp and eels.

Compounds of formula I or II the enantiomers or veterinarily acceptable salts thereof and compositions comprising them are preferably used for controlling and preventing infestations and infections in domestic animals, such as dogs or cats.

Infestations in warm-blooded animals and fish include, but are not limited to, lice, biting lice, ticks, nasal bots, keds, biting flies, muscoid flies, flies, myiasitic fly larvae, chiggers, gnats, mosquitoes and fleas.

The compounds of formula I or II or the enantiomers or veterinarily acceptable salts thereof and compositions comprising them are suitable for systemic and/or non-systemic control of ecto- and/or endoparasites. They are active against all or some stages of development.

The compounds of formula I and II are especially useful for combating ectoparasites.

The compounds of formula I and II are especially useful for combating parasites of the following orders and species, respectively:

fleas (Siphonaptera), e.g. Ctenocephalides felis, Ctenocephalides canis, Xenopsylla cheopis, Pulex irritans, Tunga penetrans, and Nosopsyllus fasciatus,
cockroaches (Blattaria—Blattodea), e.g. Blattella germanica, Blattella asahinae, Periplaneta americana, Periplaneta japonica, Periplaneta brunnea, Periplaneta fuligginosa, Periplaneta australasiae, and Blatta orientalis, flies, mosquitoes (Diptera), e.g. Aedes aegypti, Aedes albopictus, Aedes vexans, Anastrepha ludens, Anopheles maculipennis, Anopheles crucians, Anopheles albimanus, Anopheles gambiae, Anopheles freeborni, Anopheles leucosphyrus, Anopheles minimus, Anopheles quadrimaculatus, Calliphora vicina, Chrysomya bezziana, Chrysomya hominivorax, Chrysomya macellaria, Chrysops discalis, Chrysops silacea, Chrysops atlanticus, Cochliomyia hominivorax, Cordylobia anthropophaga, Culicoides furens, Culex pipiens, Culex nigripalpus, Culex quinquefasciatus, Culex tarsalis, Culiseta inornata, Culiseta melanura, Dermatobia hominis, Fannia canicularis, Gasterophilus intestinalis, Glossina morsitans, Glossina palpalis, Glossina fuscipes, Glossina tachinoides, Haematobia irritans, Haplodiplosis equestris, Hippelates spp., Hypoderma lineata, Leptoconops torrens, Lucilia caprina, Lucilia cuprina, Lucilia sericata, Lycoria pectoralis, Mansonia spp., Musca domestica, Muscina stabulans, Oestrus ovis, Phlebotomus argentipes, Psorophora columbiae, Psorophora discolor, Prosimulium mixtum, Sarcophaga haemorrhoidalis, Sarcophaga sp., Simulium vittatum, Stomoxys calcitrans, Tabanus bovinus, Tabanus atratus, Tabanus lineola, and Tabanus similis, lice (Phthiraptera), e.g. Pediculus humanus capitis, Pediculus humanus corporis, Pthirus pubis, Haematopinus eurysternus, Haematopinus suis, Linognathus vituli, Bovicola bovis, Menopon gallinae, Menacanthus stramineus and Solenopotes capillatus.
ticks and parasitic mites (Parasitiformes): ticks (Ixodida), e.g. Ixodes scapularis, Ixodes holocyclus, Ixodes pacificus, Rhiphicephalus sanguineus, Dermacentor andersoni, Dermacentor variabilis, Amblyomma americanum, Ambryomma maculatum, Ornithodorus hermsi, Ornithodorus turicata and parasitic mites (Mesostigmata), e.g. Ornithonyssus bacoti and Dermanyssus gallinae,
Actinedida (Prostigmata) and Acaridida (Astigmata) e.g. Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp., and Laminosioptes spp,

Bugs (Heteropterida): Cimex lectularius, Cimex hemipterus, Reduvius senilis, Triatoma spp., Rhodnius ssp., Panstrongylus ssp. and Arilus critatus,

Anoplurida, e.g. Haematopinus spp., Linognathus spp., Pediculus spp., Phtirus spp., and Solenopotes spp,

Mallophagida (suborders Arnblycerina and Ischnocerina), e.g. Trimenopon spp., Menopon spp., Trinoton spp., Bovicola spp., Werneckiella spp., Lepikentron spp., Trichodectes spp., and Felicola spp,

### Roundworms Nematoda:

Wipeworms and Trichinosis (Trichosyringida), e.g. Trichinellidae (Trichinella spp.), (Trichuridae) Trichuris spp., Capillaria spp,

Rhabditida, e.g. Rhabditis spp, Strongyloides spp., Helicephalobus spp,

Strongylida, e.g. Strongylus spp., Ancylostoma spp., Necator americanus, Bunostomum spp. (Hookworm), Trichostrongylus spp., Haemonchus contortus, Ostertagia spp., Cooperia spp., Nematodirus spp., Dictyocaulus spp., Cyathostoma spp., Oesophagostomum spp., Stephanurus dentatus, Ollulanus spp., Chabertia spp., Stephanurus dentatus, Syngamus trachea, Ancylostoma spp., Uncinaria spp., Globocephalus spp., Necator spp., Metastrongylus spp., Muellerius capillaris, Protostrongylus spp., Angiostrongylus spp., Parelaphostrongylus spp. Aleurostrongylus abstrusus, and Dioctophyma renale,

Intestinal roundworms (Ascaridida), e.g. Ascaris lumbricoides, Ascaris suum, Ascaridia galli, Parascaris equorum, Enterobius vermicularis (Threadworm), Toxocara canis, Toxascaris leonine, Skrjabinema spp., and Oxyuris equi,

Camallanida, e.g. Dracunculus medinensis (guinea worm)

Spirurida, e.g. Thelazia spp. Wuchereria spp., Brugia spp., Onchocerca spp., Dirofilari spp. a, Dipetalonema spp., Setaria spp., Elaeophora spp., Spirocerca lupi, and Habronema spp.,

Thorny headed worms (Acanthocephala), e.g. Acanthocephalus spp., Macracanthorhynchus hirudinaceus and Oncicola spp,

### Planarians (Plathelminthes):

Flukes (Trematoda), e.g. Faciola spp., Fascioloides magna, Paragonimus spp., Dicrocoelium spp., Fasciolopsis buski, Clonorchis sinensis, Schistosoma spp., Trichobilharzia spp., Alaria alata, Paragonimus spp., and Nanocyetes spp,

Cercomeromorpha, in particular Cestoda (Tapeworms), e.g. Diphyllobothrium spp., Tenia spp., Echinococcus spp., Dipylidium caninum, Multiceps spp., Hymenolepis spp., Mesocestoides spp., Vampirolepis spp., Moniezia spp., Anoplocephala spp., Sirometra spp., Anoplocephala spp., and Hymenolepis spp.

The compounds of formula I or II and compositions containing them are particularly useful for the control of pests from the orders Diptera, Siphonaptera and Ixodida.

Moreover, the use of the compounds of formula I or II and compositions containing them for combating mosquitoes is especially preferred.

The use of the compounds of formula I or II and compositions containing them for combating flies is a further preferred embodiment of the present invention.

Furthermore, the use of the compounds of formula I or II and compositions containing them for combating fleas is especially preferred.

The use of the compounds of formula I or II and compositions containing them for combating ticks is a further preferred embodiment of the present invention.

The compounds of formula I and II also are especially useful for combating endoparasites (roundworms nematoda, thorny headed worms and planarians).

Administration can be carried out both prophylactically and therapeutically.

Administration of the active compounds is carried out directly or in the form of suitable preparations, orally, topically/dermally or parenterally.

For oral administration to warm-blooded animals, the formula I compounds may be formulated as animal feeds, animal feed premixes, animal feed concentrates, pills, solutions, pastes, suspensions, drenches, gels, tablets, boluses and capsules. In addition, the formula I compounds may be administered to the animals in their drinking water. For oral administration, the dosage form chosen should provide the animal with 0.01 mg/kg to 100 mg/kg of animal body weight per day of the formula I or II compound, preferably with 0.5 mg/kg to 100 mg/kg of animal body weight per day.

Alternatively, the formula I or II compounds may be administered to animals parenterally, for example, by intraruminal, intramuscular, intravenous or subcutaneous injection. The formula I compounds may be dispersed or dissolved in a physiologically acceptable carrier for subcutaneous injection. Alternatively, the formula I compounds may be formulated into an implant for subcutaneous administration. In addition the formula I compound may be transdermally administered to animals. For parenteral administration, the dosage form chosen should provide the animal with 0.01 mg/kg to 100 mg/kg of animal body weight per day of the formula I compound.

The formula I or II compounds may also be applied topically to the animals in the form of dips, dusts, powders, collars, medallions, sprays, shampoos, spot-on and pour-on formulations and in ointments or oil-in-water or water-in-oil emulsions. For topical application, dips and sprays usually contain 0.5 ppm to 5,000 ppm and preferably 1 ppm to 3,000 ppm of the formula I compound. In addition, the formula I compounds may be formulated as ear tags for animals, particularly quadrupeds such as cattle and sheep.

### Suitable Preparations are:

Solutions such as oral solutions, concentrates for oral administration after dilution, solutions for use on the skin or in body cavities, pouring-on formulations, gels;
Emulsions and suspensions for oral or dermal administration; semi-solid preparations;
Formulations in which the active compound is processed in an ointment base or in an oil-in-water or water-in-oil emulsion base;
Solid preparations such as powders, premixes or concentrates, granules, pellets, tablets, boluses, capsules; aerosols and inhalants, and active compound-containing shaped articles.

Compositions suitable for injection are prepared by dissolving the active ingredient in a suitable solvent and optionally adding further ingredients such as acids, bases, buffer salts, preservatives, and solubilizers. The solutions are filtered and filled sterile.

Suitable solvents are physiologically tolerable solvents such as water, alkanols such as ethanol, butanol, benzyl alcohol, glycerol, propylene glycol, polyethylene glycols, N-methyl-pyrrolidone, 2-pyrrolidone, and mixtures thereof.

The active compounds can optionally be dissolved in physiologically tolerable vegetable or synthetic oils which are suitable for injection.

Suitable solubilizers are solvents which promote the dissolution of the active compound in the main solvent or prevent its precipitation. Examples are polyvinylpyrrolidone, polyvinyl alcohol, polyoxyethylated castor oil, and polyoxyethylated sorbitan ester.

Suitable preservatives are benzyl alcohol, trichlorobutanol, p-hydroxybenzoic acid esters, and n-butanol.

Oral solutions are administered directly. Concentrates are administered orally after prior dilution to the use concentration. Oral solutions and concentrates are prepared according to the state of the art and as described above for injection solutions, sterile procedures not being necessary.

Solutions for use on the skin are trickled on, spread on, rubbed in, sprinkled on or sprayed on.

Solutions for use on the skin are prepared according to the state of the art and according to what is described above for injection solutions, sterile procedures not being necessary.

Further suitable solvents are polypropylene glycol, phenyl ethanol, phenoxy ethanol, ester such as ethyl or butyl acetate, benzyl benzoate, ethers such as alkyleneglycol alkylether, e.g. dipropylenglycol monomethylether, ketons such as acetone, methylethylketone, aromatic hydrocarbons, vegetable and synthetic oils, dimethylformamide, dimethylacetamide, transcutol, solketal, propylencarbonate, and mixtures thereof.

It may be advantageous to add thickeners during preparation. Suitable thickeners are inorganic thickeners such as bentonites, colloidal silicic acid, aluminium monostearate, organic thickeners such as cellulose derivatives, polyvinyl alcohols and their copolymers, acrylates and methacrylates.

Gels are applied to or spread on the skin or introduced into body cavities. Gels are prepared by treating solutions which have been prepared as described in the case of the injection solutions with sufficient thickener that a clear material having an ointment-like consistency results. The thickeners employed are the thickeners given above.

Pour-on formulations are poured or sprayed onto limited areas of the skin, the active compound penetrating the skin and acting systemically.

Pour-on formulations are prepared by dissolving, suspending or emulsifying the active compound in suitable skin-compatible solvents or solvent mixtures. If appropriate, other auxiliaries such as colorants, bioabsorption-promoting substances, antioxidants, light stabilizers, adhesives are added.

Suitable solvents which are: water, alkanols, glycols, polyethylene glycols, polypropylene glycols, glycerol, aromatic alcohols such as benzyl alcohol, phenylethanol, phenoxyethanol, esters such as ethyl acetate, butyl acetate, benzyl benzoate, ethers such as alkylene glycol alkyl ethers such as dipropylene glycol monomethyl ether, diethylene glycol mono-butyl ether, ketones such as acetone, methyl ethyl ketone, cyclic carbonates such as propylene carbonate, ethylene carbonate, aromatic and/or aliphatic hydrocarbons, vegetable or synthetic oils, DMF, dimethylacetamide, n-alkylpyrrolidones such as methylpyrrolidone, n-butylpyrrolidone or n-octylpyrrolidone, N-methylpyrrolidone, 2-pyrrolidone, 2,2-dimethyl-4-oxy-methylene-1,3-diox-olane and glycerol formal.

Suitable colorants are all colorants permitted for use on animals and which can be dissolved or suspended.

Suitable absorption-promoting substances are, for example, DMSO, spreading oils such as isopropyl myristate, dipropylene glycol pelargonate, silicone oils and copolymers thereof with polyethers, fatty acid esters, triglycerides, fatty alcohols.

Suitable antioxidants are sulfites or metabisulfites such as potassium metabisulfite, ascorbic acid, butylhydroxytoluene, butylhydroxyanisole, tocopherol.

Suitable light stabilizers are, for example, novantisolic acid.

Suitable adhesives are, for example, cellulose derivatives, starch derivatives, polyacrylates, natural polymers such as alginates, gelatin.

Emulsions can be administered orally, dermally or as injections.

Emulsions are either of the water-in-oil type or of the oil-in-water type.

They are prepared by dissolving the active compound either in the hydrophobic or in the hydrophilic phase and homogenizing this with the solvent of the other phase with the aid of suitable emulsifiers and, if appropriate, other auxiliaries such as colorants, absorption-promoting substances, preservatives, antioxidants, light stabilizers, viscosity-enhancing substances.

### Suitable Hydrophobic Phases (Oils) are:

liquid paraffins, silicone oils, natural vegetable oils such as sesame oil, almond oil, castor oil, synthetic triglycerides such as caprylic/capric biglyceride, triglyceride mixture with vegetable fatty acids of the chain length C₈-C₁₂ or other specially selected natural fatty acids, partial glyceride mixtures of saturated or unsaturated fatty acids possibly also containing hydroxyl groups, mono- and diglycerides of the C₈-C₁₀ fatty acids, fatty acid esters such as ethyl stearate, di-n-butyryl adipate, hexyl laurate, dipropylene glycol perlargonate, esters of a branched fatty acid of medium chain length with saturated fatty alcohols of chain length C₁₆-C₁₈, isopropyl myristate, isopropyl palmitate, caprylic/capric acid esters of saturated fatty alcohols of chain length C₁₂-C₁₈, isopropyl stearate, oleyl oleate, decyl oleate, ethyl oleate, ethyl lactate, waxy fatty acid esters such as synthetic duck coccygeal gland fat, dibutyl phthalate, diisopropyl adipate, and ester mixtures related to the latter, fatty alcohols such as isotridecyl alcohol, 2-octyldodecanol, cetylstearyl alcohol, oleyl alcohol, and fatty acids such as oleic acid and mixtures thereof.

Suitable hydrophilic phases are: water, alcohols such as propylene glycol, glycerol, sorbitol and mixtures thereof.

### Suitable Emulsifiers are:

non-ionic surfactants, e.g. polyethoxylated castor oil, polyethoxylated sorbitan monooleate, sorbitan monostearate, glycerol monostearate, polyoxyethyl stearate, alkylphenol polyglycol ether;
ampholytic surfactants such as di-sodium N-lauryl-p-iminodipropionate or lecithin; anionic surfactants, such as sodium lauryl sulfate, fatty alcohol ether sulfates, mono/dialkyl polyglycol ether orthophosphoric acid ester monoethanolamine salt; cation-active surfactants, such as cetyltrimethylammonium chloride.

Suitable further auxiliaries are: substances which enhance the viscosity and stabilize the emulsion, such as carboxymethylcellulose, methylcellulose and other cellulose and starch derivatives, polyacrylates, alginates, gelatin, gum arabic, polyvinylpyrrolidone, polyvinyl alcohol, copolymers of methyl vinyl ether and maleic anhydride, polyethylene glycols, waxes, colloidal silicic acid or mixtures of the substances mentioned.

Suspensions can be administered orally or topically/dermally. They are prepared by suspending the active compound in a suspending agent, if appropriate with addition of other auxiliaries such as wetting agents, colorants, bioabsorption-promoting substances, preservatives, antioxidants, light stabilizers.

Liquid suspending agents are all homogeneous solvents and solvent mixtures.

Suitable wetting agents (dispersants) are the emulsifiers given above.

Other auxiliaries which may be mentioned are those given above.

Semi-solid preparations can be administered orally or topically/dermally. They differ from the suspensions and emulsions described above only by their higher viscosity.

For the production of solid preparations, the active compound is mixed with suitable excipients, if appropriate with addition of auxiliaries, and brought into the desired form.

Suitable excipients are all physiologically tolerable solid inert substances. Those used are inorganic and organic substances. Inorganic substances are, for example, sodium chloride, carbonates such as calcium carbonate, hydrogencarbonates, aluminium oxides, titanium oxide, silicic acids, argillaceous earths, precipitated or colloidal silica, or phosphates. Organic substances are, for example, sugar, cellulose, foodstuffs and feeds such as milk powder, animal meal, grain meals and shreds, starches.

Suitable auxiliaries are preservatives, antioxidants, and/or colorants which have been mentioned above.

Other suitable auxiliaries are lubricants and glidants such as magnesium stearate, stearic acid, talc, bentonites, disintegration-promoting substances such as starch or crosslinked polyvinylpyrrolidone, binders such as starch, gelatin or linear polyvinylpyrrolidone, and dry binders such as microcrystalline cellulose.

In general, “parasiticidally effective amount” means the amount of active ingredient needed to achieve an observable effect on growth, including the effects of necrosis, death, retardation, prevention, and removal, destruction, or otherwise diminishing the occurrence and activity of the target organism. The parasiticidally effective amount can vary for the various compounds/compositions used in the invention. A parasiticidally effective amount of the compositions will also vary according to the prevailing conditions such as desired parasiticidal effect and duration, target species, mode of application, and the like.

The compositions which can be used in the invention can comprise generally from about 0.001 to 95% of the compound of formula I.

Generally it is favorable to apply the compounds of formula I in total amounts of 0.5 mg/kg to 100 mg/kg per day, preferably 1 mg/kg to 50 mg/kg per day.

Ready-to-use preparations contain the compounds acting against parasites, preferably ectoparasites, in concentrations of 10 ppm to 80 percent by weight, preferably from 0.1 to 65 percent by weight, more preferably from 1 to 50 percent by weight, most preferably from 5 to 40 percent by weight.

Preparations which are diluted before use contain the compounds acting against ecto-parasites in concentrations of 0.5 to 90 percent by weight, preferably of 1 to 50 percent by weight.

Furthermore, the preparations comprise the compounds of formula I against endoparasites in concentrations of 10 ppm to 2 percent by weight, preferably of 0.05 to 0.9 percent by weight, very particularly preferably of 0.005 to 0.25 percent by weight.

In a preferred embodiment of the present invention, the compositions comprising the compounds of formula I them are applied dermally/topically.

In a further preferred embodiment, the topical application is conducted in the form of compound-containing shaped articles such as collars, medallions, ear tags, bands for fixing at body parts, and adhesive strips and foils.

Generally it is favorable to apply solid formulations which release compounds of formula I in total amounts of 10 mg/kg to 300 mg/kg, preferably 20 mg/kg to 200 mg/kg, most preferably 25 mg/kg to 160 mg/kg body weight of the treated animal in the course of three weeks.

For the preparation of the shaped articles, thermoplastic and flexible plastics as well as elastomers and thermoplastic elastomers are used. Suitable plastics and elastomers are polyvinyl resins, polyurethane, polyacrylate, epoxy resins, cellulose, cellulose derivatives, polyamides and polyester which are sufficiently compatible with the compounds of formula I. A detailed list of plastics and elastomers as well as preparation procedures for the shaped articles is given e.g. in WO 03/086075.

The present invention is now illustrated in further details by the following examples.

### A) Preparation Examples

The compounds of formulae I and II were characterized by coupled High Performance Liquid Chromatography/mass spectroscopy (HPLC/MS), by NMR or by their melting points. HPLC: RP-18 column (Chromolith Speed ROD from Merck KgaA, Germany). Elution: acetonitrile+0.1% trifluoroacetic acid (TFA)/water+0.1% TFA in a ratio of from 5:95 to 95:5 in 5 minutes at 40° C. MS: Quadrupol electrospray ionisation, 80 V (positive modus).

### A.1) Preparation of Compounds of Formula II

### EXAMPLE 1

### Preparation of acetic acid 2-{3-[1-(2,3-dimethyl-phenyl)-4-m-tolyl-but-3-ynyl]-thioureido}-ethyl ester

A solution of 1-(2,3-dimethyl-phenyl)-4-m-tolyl-but-3-ynylamine (2.00 g, 7.59 mmol, 1 eq, cf. example 24.3) and acetic acid 2-isothiocyanatoethylester (1.10 g, 7.59 mmol, 1 eq, prepared according to Collect. Czech. Chem. Commun. 1986, 51, 112) in tetrahydrofuran (100 ml) was stirred at 60° C. for 4 h. The obtained reaction mixture was concentrated in vacuo and the residue was purified by column chromatography to yield acetic acid 2-{3-[1-(2,3-dimethyl-phenyl)-4-m-tolyl-but-3-ynyl]-thioureido}-ethyl ester.

### EXAMPLE 2

### Preparation of 1-[1-(2,3-dimethyl-phenyl)-4-m-tolyl-but-3-ynyl]-3-(2-hydroxy-ethyl)-thiourea

A mixture of acetic acid 2-{3-[1-(2,3-dimethyl-phenyl)-4-m-tolyl-but-3-ynyl]-thioureido}-ethyl ester (1.02 g, 2.50 mmol, 1 eq, cf. example 1), lithium hydroxide monohydrate (210 mg, 4.99 mmol, 2 eq) in water (5 ml) and tetrahydrofuran (50 ml) was stirred for 18 h. Water was then added and the obtained mixture was extracted with dichloromethane (4×). The combined organic extracts were dried over sodium sulfate, filtered and concentrated in vacuo to yield 1-[1-(2,3-dimethyl-phenyl)-4-m-tolyl-but-3-ynyl]-3-(2-hydroxy-ethyl)-thiourea.

Urea compounds (i.e. compounds of formula II, wherein X is O) can be prepared in analogy to the method outlined in preparation example 1 by using isocyanato compounds instead of isothiocyanato compounds.

The compounds of formula (II)′ shown in table 2 hereinafter (examples 3 to 12)

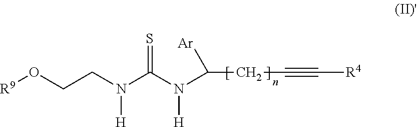

wherein n, Ar, R⁴, and R⁹ have the meanings given in table 2 and have been prepared in analogy to the preparation examples shown hereinbefore.

TABLE 2

| | | | | | Physico-chemical data |
|---|---|---|---|---|---|
| | | | | | (m.p.; ¹H-NMR (CDCl₃); |
| Ex. | n | Ar | R⁴ | R⁹ | HPLC; MS) |
| 1 | 1 | 2,3-(CH₃)₂—C₆H₃ | 3-CH₃—C₆H₄ | C(═O)CH₃ | tr = 3.89, m/z = 409 |
| 2 | 1 | 2,3-(CH₃)₂—C₆H₃ | 3-CH₃—C₆H₄ | H | tr = 3.70, m/z = 367 |
| 3 | 1 | 2,3-(CH₃)₂—C₆H₃ | C₆H₅ | C(═O)CH₃ | tr = 3.83, m/z = 395 |
| 4 | 1 | 2,3-(CH₃)₂—C₆H₃ | C₆H₅ | H | tr = 3.33, m/z = 353 |
| 5 | 1 | 2,3-(CH₃)₂—C₆H₃ | 2-CH₃—C₆H₄ | C(═O)CH₃ | tr = 3.81, m/z = 409 |
| 6 | 1 | 2,3-(CH₃)₂—C₆H₃ | 2-CH₃—C₆H₄ | H | tr = 3.61, m/z = 367 |
| 7 | 1 | 2,3-(CH₃)₂—C₆H₃ | 4-CH₃—C₆H₄ | C(═O)CH₃ | tr = 3.89, m/z = 409 |
| 8 | 1 | 2,3-(CH₃)₂—C₆H₃ | 3,5-(CH₃)₂—C₆H₃ | C(═O)CH₃ | 1.91 (s, 3H), 2.27 (s, |
| | | | | | 6H), 2.31 (s, 3H), |
| | | | | | 2.33 (s, 3H), 2.79-3.05 (m, |
| | | | | | 2H), 3.65-3.78 (m, 2H), |
| | | | | | 4.06-4.14 (m, 2H), |
| | | | | | 5.42 (bs, 1H), 6.01 (bs, 1H), |
| | | | | | 6.72 (m, 1H), 6.90 (s, |
| | | | | | 1H), 6.96 (s, 2H), |
| | | | | | 7.11 (m, 2H), 7.29 (m, 1H) |
| 9 | 1 | 2,3-(CH₃)₂—C₆H₃ | 4-CH₃—C₆H₄ | H | tr = 3.70, m/z = 367 |
| 10 | 1 | 2,3-(CH₃)₂—C₆H₃ | 3,5-(CH₃)₂—C₆H₃ | H | tr = 3.90, m/z = 381 |
| 11 | 1 | 2,3-(CH₃)₂—C₆H₃ | 3-OCH₃—C₆H₄ | C(═O)CH₃ | tr = 3.81, m/z = 425 |
| 12 | 1 | 2,3-(CH₃)₂—C₆H₃ | 3-OCH₃—C₆H₄ | H | tr = 3.48, m/z = 383 |

HPLC-MS data are reported as retention time (tᵣ, [min]) and detected mass [m/z];
¹H-NMR(CDCl₃) data are reported as chemical shift δ [ppm];
m.p. = melting point [° C.].

### A.2) Preparation of Compounds of Formula I

### EXAMPLE 13

### Preparation of (4,5-dihydro-thiazol-2-yl)-[1-(2,3-dimethyl-phenyl)-4-m-tolyl-but-3-ynyl]-amine

Diisopropylethylamine (0.48 ml, 2.82 mmol, 1.5 eq) and cyanomethyl-trimethyl-phosphonium iodide (915 mg, 3.77 mmol, 2 eq, cf. Tetrahedron 2001, 57, 5451) were successively added to a solution of 1-[1-(2,3-dimethyl-phenyl)-4-m-tolyl-but-3-ynyl]-3-(2-hydroxy-ethyl)-thiourea (690 mg, 1.88 mmol, 1 eq, cf. example 2) in propionitrile (15 ml). After stirring at 90° C. for 4 h, the mixture was concentrated in vacuo and the residue was dissolved in ethyl acetate. The mixture was then washed with a saturated aqueous solution of ammonium chloride and with a saturated aqueous solution of sodium hydrogen carbonate. The organic phase was dried over sodium sulfate, filtered and concentrated in vacuo. The residue was purified by column chromatography to yield (4,5-dihydro-thiazol-2-yl)-[1-(2,3-dimethyl-phenyl)-4-m-tolyl-but-3-ynyl]amine.

### EXAMPLE 14

### Preparation of (4,5-dihydro-thiazol-2-yl)-[1-(2,3-dimethyl-phenyl)-but-3-ynyl]-amine

A solution of 2-chloroethyl isothiocyanate (702 mg, 5.77 mmol, 1 eq.) was added dropwise to a solution of 1-(2,3-dimethylphenyl)-but-3-ynylamine (1.00 g, 5.77 mmol, 1 eq., cf. example 23) in Et₂O (20 mL) at 0° C. After stirring for 3 h, 1 M aqueous NaOH was added, the layers were separated and the aqueous phase extracted with Et₂O. The combined organic phases were washed with H₂O, dried over Na₂SO₄ and concentrated in vacuo. The residue was purified by column chromatography to yield (4,5-dihydro-thiazol-2-yl)-[1-(2,3-dimethyl-phenyl)-but-3-ynyl]-amine.

### EXAMPLE 15

### Preparation of [4-(3-chloro-phenyl)-1-(2,3-dimethyl-phenyl)-but-3-ynyl]-(4,5-dihydro-thiazol-2-yl)-amine

A mixture of (4,5-dihydro-thiazol-2-yl)-[1-(2,3-dimethyl-phenyl)-but-3-ynyl]-amine (500 mg, 1.94 mmol, 1 eq., cf. example 14), 1-chloro-3-iodobenzene (554 mg, 2.32 mmol, 1.2 eq.), diethylamine (696 mg, 9.68 mmol, 5 eq.), copper(I) iodide (36 mg, 0.19 mmol, 0.1 eq.) and bis(triphenylphosphine)palladium(II) dichloride (74 mg, 0.10 mmol, 0.05 eq.) in diethyl ether (20 ml) was stirred at room temperature for 4 h. The mixture was quenched with saturated aqueous ammonium chloride, the phases separated and the aqueous phase extracted with diethyl ether. The combined organic phases were washed with H₂O, dried over Na₂SO₄ and concentrated in vacuo. The residue was purified by column chromatography to yield [4-(3-chloro-phenyl)-1-(2,3-dimethyl-phenyl)-but-3-ynyl]-(4,5-dihydro-thiazol-2-yl)-amine.

The compounds of formula (I)′ shown in table 3 hereinafter (examples 13 to 22)

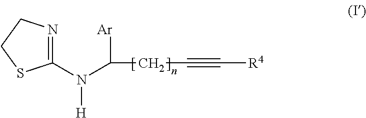

wherein n, Ar, and R⁴ have the meanings given in table 3 and have been prepared in analogy to the preparation examples shown hereinbefore.

TABLE 3

| | | | | Physico-chemical |
|---|---|---|---|---|
| | | | | data (m.p.; |
| | | | | ¹H-NMR; |
| | | | | ¹³C-NMR; HPLC- |
| Ex. | n | Ar | R⁴ | MS) |
| 13 | 1 | 2,3-(CH₃)₂—C₆H₃ | 3-CH₃—C₆H₄ | tr = 3.14, m/z = 349 |
| 14 | 1 | 2,3-(CH₃)₂—C₆H₃ | H | tr = 2.23, m/z = 259 |
| 15 | 1 | 2,3-(CH₃)₂—C₆H₃ | 3-Cl—C₆H₄ | tr = 3.20, m/z = 369 |
| 16 | 1 | 2,3-(CH₃)₂—C₆H₃ | 4-Cl—C₆H₄ | tr = 3.20, m/z = 369 |
| 17 | 1 | 2,3-(CH₃)₂—C₆H₃ | C₆H₅ | tr = 2.99, m/z = 335 |
| 18 | 1 | 2,3-(CH₃)₂—C₆H₃ | 2-CH₃—C₆H₄ | tr = 3.27, m/z = 349 |
| 19 | 1 | 2,3-(CH₃)₂—C₆H₃ | 4-CH₃—C₆H₄ | tr = 3.15, m/z = 349 |
| 20 | 1 | 2,3-(CH₃)₂—C₆H₃ | 3,5-(CH₃)₂—C₆H₃ | tr = 3.35, m/z = 363 |
| 21 | 1 | 2,3-(CH₃)₂—C₆H₃ | 3-OCH₃—C₆H₄ | tr = 3.02, m/z = 365 |
| 22 | 1 | 2,3-(CH₃)₂—C₆H₃ | 2-Cl—C₆H₄ | tr = 3.06, m/z = 369 |

HPLC-MS data are reported as retention time (tᵣ, [min]) and detected mass [m/z];
¹H-NMR(CDCl₃) data are reported as chemical shift δ [ppm];
¹³C-NMR(CDCl₃) data are reported as chemical shift δ [ppm];
m.p. = melting point [° C.].

### A.3) Preparation of Starting Compounds of Formula III

### EXAMPLE 23

### Preparation of 1-(2,3-dimethylphenyl)-but-3-ynylamine

Preparation of solution A: 1,2-dibromoethane (12.9 g, 69.0 mmol, 0.12 eq) was added to zinc (123 g, 1.89 mol, 3.3 eq) in tetrahydrofuran (80 mL) and the mixture was refluxed for 1 h. A solution of chlorotrimethylsilane (7.50 g, 69.0 mmol, 0.12 eq) in tetrahydrofuran (165 mL) was then added at room temperature followed by the dropwise addition of propargylbromide (204 g, 1.71 mol, 3 eq) in tetrahydrofuran (165 mL) at −10° C. The resulting mixture was then stirred at −10° C. for 1.5 h.

Preparation of solution B: in a separate flask, lithium bis(trimethylsilyl)amide (1 M in tetrahydrofuran, 629 mL, 629 mmol, 1.1 eq) was added dropwise to a solution of 2,3-dimethylbenzaldehyde (76.5 g, 571 mmol, 1 eq) in tetrahydrofuran (80 mL) at 0° C. The solution was then stirred at this temperature for 15 min and then 1 h at room temperature. Solution B was then added dropwise to solution A at −10° C. and the mixture was allowed to warm up to room temperature overnight. A saturated aqueous solution of K₂CO₃ (350 mL) was then added at 0° C., followed by water (1.3 L) and methyl-tert-butyl ether (500 mL). The phases were separated, and the aqueous portion was further extracted with methyl-tert-butyl ether (5×500 mL). The combined organic extracts were concentrated in vacuo. The residue was dissolved in methyl-tert-butyl ether (200 mL) and the solution was washed subsequently with water (2×200 mL) and saturated aqueous sodium chloride, dried over sodium sulfate, filtered and concentrated in vacuo to yield 1-(2,3-dimethylphenyl)-but-3-ynylamine in an amount of 103 g.

### EXAMPLE 24

### Preparation of 1-(2,3-dimethyl-phenyl)-4-m-tolyl-but-3-ynylamine

### 24.1 Preparation of [1-(2,3-dimethyl-phenyl)-but-3-ynyl]-carbamic acid tert-butyl ester

A mixture of 1-(2,3-dimethylphenyl)-but-3-ynylamine (4.00 g, 23.1 mmol, 1 eq, cf. example 23) and di-tert-butyl-dicarbonate (5.04 g, 23.1 mmol, 1 eq) and triethylamine (7 ml, 50.8 mmol, 2.2 eq) in dichloromethane (60 mL) was stirred overnight. The mixture was washed with water and saturated aqueous sodium chloride, dried over sodium sulfate, filtered and concentrated in vacuo. The residue was purified by column chromatography to yield [1-(2,3-dimethyl-phenyl)-but-3-ynyl]-carbamic acid tert-butyl ester.

### 24.2 Preparation of [1-(2,3-dimethyl-phenyl)-4-m-tolyl-but-3-ynyl]-carbamic acid tert-butyl ester

A mixture of [1-(2,3-dimethyl-phenyl)-but-3-ynyl]-carbamic acid tert-butyl ester (5.00 g, 18.3 mmol, 1 eq., cf. example 24.1), 3-iodotoluene (4.55 g, 22.0 mmol, 1.2 eq.), di-ethylamine (9.6 ml, 91.4 mmol, 5 eq.), copper(I) iodide (350 mg, 1.83 mmol, 0.1 eq.) and bis(triphenylphosphine)palladium(II) dichloride (670 mg, 0.91 mmol, 0.05 eq.) in diethyl ether (200 ml) was stirred at room temperature for 3 h. The mixture was quenched with saturated aqueous ammonium chloride, the phases separated and the aqueous phase extracted with diethyl ether. The combined organic phases were washed with H2O, dried over Na₂SO₄ and concentrated in vacuo. The residue was purified by column chromatography to yield [1-(2,3-dimethyl-phenyl)-4-m-tolyl-but-3-ynyl]-carbamic acid tert-butyl ester.

### 24.3 Preparation of 1-(2,3-dimethyl-phenyl)-4-m-tolyl-but-3-ynylamine

A mixture of [1-(2,3-dimethyl-phenyl)-4-m-tolyl-but-3-ynyl]-carbamic acid tert-butyl ester (3.00 g, 8.25 mmol, 1 eq., cf. example 24.2) and hydrochloric acid (4 N in dioxane, 9.6 ml, 38.8 mmol) in dioxane (100 ml) was stirred for 4 h at 40° C. A 25% aqueous solution of ammonia was added until pH 10. The mixture was then extracted with dichloromethane, the combined organic extracts were dried over Na₂SO₄ and concentrated in vacuo. The residue was purified by column chromatography to yield 1-(2,3-dimethylphenyl)-4-m-tolyl-but-3-ynylamine.

### B) Biological Examples

### Action Against Pests:

The action of the compounds of the general formulae I and II against pests was evaluated by the following experiments:

I. Cotton Aphid (Aphis gossypii)

The active compounds were formulated in 50:50 acetone: water and 100 ppm Kinetic® surfactant.

Cotton plants in the cotyledon stage (variety ‘Delta Pine’) are infested with approximately 100 laboratory-reared aphids by placing infested leaf sections on top of the test plants. The leaf sections are removed after 24 hours. The cotyledons of the intact plants are dipped into gradient solutions of the test compound. Aphid mortality on the treated plants, relative to mortality on check plants, is determined after 5 days.

In this test, the compounds of examples 5, 6, 8, 13, 15, 16, 17, 18, 19, 20 at 300 ppm showed over 70% mortality in comparison with untreated controls.

II. Green Peach Aphid (Myzus persicae)

The active compounds were formulated in 50:50 acetone: water and 100 ppm Kinetic® surfactant.

Pepper plants in the 2nd leaf-pair stage (variety ‘California Wonder’) are infested with approximately 40 laboratory-reared aphids by placing infested leaf sections on top of the test plants. The leaf sections are removed after 24 hours. The leaves of the intact plants are dipped into gradient solutions of the test compound. Aphid mortality on the treated plants, relative to mortality on check plants, is determined after 5 days.

In this test, the compounds of examples 4, 5, 6, 7, 11, 13, 15, 16, 17, 18, 19, 20 at 300 ppm showed over 70% mortality in comparison with untreated controls.

III. Cowpea Aphid (Aphis craccivora)

The active compounds were formulated in 50:50 acetone: water and 100 ppm Kinetic® surfactant.

Potted cowpea plants colonized with 100-150 aphids of various stages were sprayed after the pest population has been recorded. Population reduction was recorded after 24, 72, and 120 hours.

In this test, the compounds of examples 2, 5, 6, 12, 13, 14, 15, 16, 17, 18, 19, 20 at 300 ppm showed over 70% mortality in comparison with untreated controls.

### IV. Silverleaf Whitefly

The active compounds were formulated in 50:50 acetone: water and 100 ppm Kinetic® surfactant.

Selected cotton plants were grown to the cotyledon state (one plant per pot). The cotyledons were dipped into the test solution to provide complete coverage of the foliage and placed in a well-vented area to dry. Each pot with treated seedling was placed in a plastic cup and 10 to 12 whitefly adults (approximately 3-5 day old) were introduced. The insects were colleted using an aspirator and a 0.6 cm, non-toxic Tygon® tubing (R-3603) connected to a barrier pipette tip. The tip, containing the collected insects, was then gently inserted into the soil containing the treated plant, allowing insects to crawl out of the tip to reach the foliage for feeding. The cups were covered with a re-usable screened lid (150 micron mesh polyester screen PeCap from Tetko Inc). Test plants were maintained in the holding room at about 25 oC and 20-40% relative humidity for 3 days avoiding direct exposure to the fluorescent light (24 hour photoperiod) to prevent trapping of heat inside the cup. Mortality was assessed 3 days after treatment of the plants.

In this test, the compound of example 13 at 300 ppm showed over 70% mortality in comparison with untreated controls.

1-26. (canceled)

27. An azolin-2-ylamino compound of formula (I), 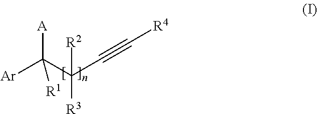
wherein
n is 0,1, 2 or 3;
Ar is phenyl, which is unsubstituted or carries 1, 2, 3, 4 or 5 substituents R^{Ar}, wherein
R^{Ar} is selected from the group consisting of halogen, CN, N₃, NO₂, C₁-C₆-alkyl, C₂-C₆-alkenyl and C₂-C₆-alkynyl, wherein each carbon atom in the aforementioned radicals may carry any combination of 1, 2 or 3 substituents, independently of one another selected from the group consisting of halogen, CN, NO₂, NH₂, OH, SH, COOH, C₁-C₆-alkoxy, C₂-C₆-alkenyloxy, C₂-C₆-alkynyloxy, C₁-C₆-haloalkoxy and C₁-C₆-alkylthio, and
R^{Ar} may further be selected from the group consisting of —C(═O)R^{a}, —C(═S)R^{a}, —C(═NR^{f})R^{a}, —C(═NR^{f})OR^{b}, —C(═NR^{f})NR^{c}R^{d}, —C(═NR^{f})SR^{e}, —C(═O)OR^{b}, —C(═O)NR^{c}R^{d}, —C(═O)SR^{e}, —C(═S)OR^{b}, —C(═S)NR^{c}R^{d}, —C(═S)SR^{e}, —OR^{b}, —O—C(═O)R^{a}, —O—C(═O)OR^{b}, —O—C(═O)—NR^{c}R^{d}, —O—C(═O)SR^{e}, —SR^{e}, —S(═O)R^{e}, —S(═O)₂R^{e}, —S(═O)₂OR^{b}, —S(═O)₂NR^{c}R^{d}, —NR^{c}R^{d}, —NR^{f}—C(═O)R^{a}, —NR^{f}—C(═O)OR^{b}, —NR^{f}—C(═O)NR^{c}R^{d}, —N═CR^{a}R^{a}, —NR^{f}—NR^{c}R^{d}, —NR^{f}—C(═O)SR^{e} and —NR^{f}—C(═S)NR^{c}R^{d}, wherein R^{a}, R^{b}, R^{c}, R^{d}, R^{e} and R^{f} independently of one another have one of the meanings given below, and
R^{Ar} may further be selected from cyclic radicals —Y-Cy and —Y—Ar¹, wherein Ar¹ has one of the meanings given below,
Y is a single bond, C₁-C₆-alkandiyl, C₁-C₆-alkandiyloxy, —C(═O)—, —C(═S)—, —C(═NR^{f})—, —C(═NR^{f})O—, —C(═NR^{f})S—, —C(═NR^{f})NR^{c}, —C(═O)O—, —C(═O)NR^{c}—, —C(═O)S—, —C(═S)O—, —C(═S)NR^{e}—, —C(═S)S—, —O—, —O—C(═O)—, —O—C(═O)O—, —O—C(═O)—NR^{c}—, —O—C(═O)S—, —S—, —S(═O)—, —S(═O)₂—, —S(═O)₂O—, —S(═O)₂NR^{c}—, —NR^{f}—C(═O)—, —NR^{f}—C(═O)O—, —NR^{f}—C(═O)NR^{c}—, —N═CR^{a}—, —NR^{f}—NR^{c}—, —NR^{f}—C(═O)S—, or —NR^{f}—C(═S)NR^{c}—, wherein R^{a}, R^{b}, R^{c} and R^{f} independently of one another have one of the meanings given below,
Cy is C₃-C₁₂-cycloalkyl or a saturated or partially unsaturated 5, 6 or 7-membered heterocycle containing 1, 2, 3 or 4 heteroatoms selected independently from one another from the group consisting of S, O and N as ring members, wherein C₃-C₁₂-cycloalkyl and the heterocycle are each unsubstituted or carry any combination of 1, 2, 3, 4 or 5 substituents, independently of one another selected from the group consisting of halogen, halogen, CN, NO₂, OH, SH, NH₂, COOH, C₁-C₆-alkyl, C₁-C₆-haloalkyl, C₁-C₆-alkoxy, C₂-C₆-alkenyloxy, C₂-C₆-alkynyloxy, C₁-C₆-haloalkoxy and C₁-C₆-alkylthio,
and/or two radicals R^{Ar} bound to adjacent carbon atoms of the phenyl ring together with said carbon atoms form a fused benzene, a fused saturated or partially unsaturated 5-, 6- or 7-membered carbocycle or a fused 5-, 6- or 7-membered heterocycle, which contains 1, 2, 3 or 4 heteroatoms selected from the group consisting of O, S and N as ring members, wherein each fused benzene, carbocycle or heterocycle is unsubstituted or carries 1, 2, 3 or 4 substituents R^{Ar} as defined above with the exception that such two radicals R^{Ar} when bound to adjacent carbon atoms of the fused benzene, carbocycle or heterocycle do not form another fused ring system;
A is a radical of the formulae A.1 or A.2, 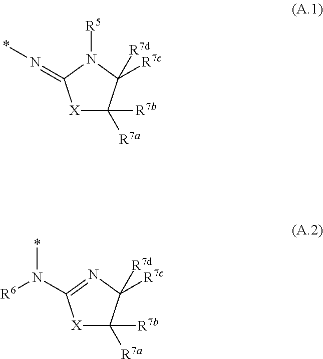
wherein
* indicates the point of attachment to the remaining part of the compound;
X is S, O or N(R⁸);
R^{7a}, R^{7b}, R^{7c} and R^{7d} are selected independently from one another from the group consisting of hydrogen, halogen, C₁-C₆-alkyl, C₁-C₆-alkoxy, C₁-C₆-alkylamino and C₃-C₆-cycloalkyl, wherein
each carbon atom of the aforementioned radicals may be unsubstituted or carries any combination of 1, 2 or 3 substituents, selected independently of one another from the group consisting of halogen, CN, NO₂, —OR^{b}, —SR^{e}, —NR^{c}R^{d}, C(═O)—R^{a}, C(═O)OR^{b} and Ar², wherein Ar², R^{a}, R^{b}, R^{c}, R^{d} and R^{e} have one of the meanings given below and wherein C₃-C₆-cycloalkyl additionally may carry any combination of 1, 2 or 3 substituents independent of one another selected from the group consisting of C₁-C₆-alkyl, C₂-C₆-alkenyl, C₂-C₆-alkynyl and C₁-C₆-haloalkyl,
R⁵, R⁶ and R⁸ are each selected independently from one another from the group consisting of hydrogen, CN, NO₂, C₁-C₆-alkyl, C₂-C₆-alkenyl, C₂-C₆-alkynyl, C₃-C₈-cycloalkyl and Ar³, wherein C₁-C₆-alkyl, C₂-C₆-alkenyl, C₂-C₆-alkynyl and C₃-C₈-cycloalkyl are unsubstituted or carry any combination of 1, 2, 3, 4 or 5 substituents, selected independently of one another from the group consisting of halogen, CN, NO₂, —OR^{b}, —NR^{c}R^{d}, —SR^{e}, —C(═O)R^{a}, —C(═O)OR^{b}, saturated or partially unsaturated 5, 6 or 7-membered heterocycles containing 1, 2, 3 or 4 heteroatoms selected independently from one another from the group consisting of S, O and N as ring members, —Ar⁴, —O-AR⁴, —S—Ar⁴ and —CH₂—Ar⁴, wherein Ar³, Ar⁴, R^{a}, R^{b}, R^{c}, R^{d}, Rₑ and R^{f} have one of the meanings given below, and wherein C₃-C₈-cycloalkyl and the heterocycles additionally may carry any combination of 1, 2, or 3 substituents, selected independently of one another from the group consisting of C₁-C₆-alkyl, C₁-C₆-haloalkyl, C₂-C₆-alkenyl and C₂-C₆-alkynyl, and
R⁵, R⁶ and R⁸ may further be selected from the group consisting of -E-C(═O)R^{a}, -E-C(═S)R^{a}, -E-C(═NR^{f})R^{a}, -E-C(═NR^{f})OR^{b}, -E-C(═NR^{f})NR^{c}R^{d}, -E-C(═NR^{f})SR^{e}, -E-C(═O)OR^{b}, -E-C(═O)NR^{c}R^{d}, -E-C(═O)SR^{e}, -E-C(═S)OR^{b}, -E-C(═S)NR^{c}R^{d}, -E-C(═S)SR^{e}, -E-OR^{b}, -E-O—C(═O)R^{a}, -E-O—C(═O)OR^{b}, -E-O—C(═O)—NR^{c}R^{d}, -E-O—C(═O)SR^{e}, -E-NR^{c}R^{d}, -E-NR^{f}—C(═O)R^{a}, -E-NR^{f}—C(═O)OR^{b}, -E-NR^{f}—C(═O)NR^{c}R^{d}, -E-N═CR^{a}R^{a′}, -E-NR^{f}—NR^{c}R^{d}, -E-NR^{f}—C(═O)SR^{e}, -E-NR^{f}—C(═S)NR^{c}R^{d}, -E-S(═O)R^{e}, -E-S(═O)₂R^{e}, —S(═O)₂OR^{b}, —S(═O)₂NR^{c}R^{d}, —P(═O)R^{g}R^{h} and P(═S)R^{g}R^{h}, wherein
E is a single bond or linear or branched C₁-C₄-alkanediyl, and
R^{a}, R^{a′}, R^{b}, R^{c}, R^{d}, R^{e}, R^{f}, R^{g} and R^{h} independently of one another have one of the meanings given below;
R¹ is selected from the group consisting of hydrogen, halogen, C₁-C₆-alkyl, C₂-C₆-alkenyl, C₂-C₆-alkynyl, C₃-C₆-cycloalkyl, phenyl and benzyl, wherein each carbon atom of the aforementioned radicals is unsubstituted or carries any combination of 1, 2 or 3 substituents selected independently of one another from the group consisting of halogen, CN, NO₂, —OR^{b}, NR^{c}R^{d}, —SR^{e}, —C(═O)R^{a} and —C(═O)OR^{b}, wherein R^{a}, R^{b}, R^{c}, R^{d} and R^{e} have one of the meanings given below, and wherein
C₃-C₆-cycloalkyl, phenyl and benzyl additionally may carry 1, 2, 3, 4 or 5 substituents selected independently of one another from the group consisting of C₁-C₆-alkyl, C₂-C₆-alkenyl, C₂-C₆-alkynyl and C₁-C₆-haloalkyl;
R² and R³ are each selected independently of one another from the group consisting of hydrogen, halogen, C₁-C₆-alkyl, C₂-C₆-alkenyl, C₂-C₆-alkynyl, C₃-C₆-cycloalkyl, phenyl and benzyl, wherein each carbon atom of the aforementioned radicals is unsubstituted or carries any combination of 1, 2 or 3 radicals selected independently of one another from the group consisting of halogen, CN, NO₂, —OR^{b}, NR^{c}R^{d}, —C(═O)R^{a} and —C(═O)OR^{b}, wherein R^{a}, R^{b}, R^{c}, R^{d} and R^{e} have one of the meanings given below, and wherein C₃-C₆-cycloalkyl, phenyl and benzyl additionally may carry 1, 2, 3, 4 or 5 substituents selected independently of one another from the group consisting of C₁-C₆-alkyl, C₂-C₆-alkenyl, C₂-C₆-alkynyl and C₁-C₆-haloalkyl,
wherein, when n is 2 or 3, the respective 2 or 3 substituents R² and 2 or 3 substituents R³ of this azolin-2-ylamino compound of formula I are selected independently from one another,
R⁴ is selected from the group consisting of hydrogen, halogen, CN, C₁-C₆-alkyl, C₂-C₆-alkenyl, C₂-C₆-alkynyl, C₃-C₈-cycloalkyl, saturated or partially unsaturated 5, 6 or 7-membered heterocycle containing 1, 2, 3 or 4 heteroatoms selected independently from one another from the group consisting of O, S and N as ring members, and Ar⁵,
wherein C₁-C₆-alkyl, C₂-C₆-alkenyl, C₂-C₆-alkynyl, C₃-Cₛ-cycloalkyl and the heterocycle are unsubstituted or carry any combination of 1, 2, 3, 4 or 5 substituents, selected independently of one another from the group consisting of halogen, CN, NO₂, —OR^{b}, —NR^{c}R^{d}, —SR^{e}, —C(═O)R^{a}, —C(═O)OR^{b}, Ar⁶, —CH₂—Ar⁶, —O—Ar⁶, and —S—Ar⁶, wherein R^{b}, R^{c}, R^{d}R^{e}, Ar⁵ and Ar⁶ have one of the meanings given below, and wherein C₃-C₈-cycloalkyl and the heterocycle additionally may carry 1, 2, 3, 4 or 5 substituents selected independently of one another from the group consisting of C₁-C₆-alkyl, C₂-C₆-alkenyl, C₂-C₆-alkynyl and C₁-C₆-haloalkyl, and
R⁴ may further be selected from the group consisting of —SiR^{a}R^{b}R^{c}, —Si(OR^{a})R^{b}R^{c}, —Si(OR^{a})(OR^{b})R^{c}, —Si(OR^{a})(OR^{b})(OR^{c}), —Si(—NR^{a}R^{b})R^{c}R^{d}, —C(═O)R^{a}, —C(═S)R^{a}, —C(═NR^{f})R^{a}, —C(═NR^{f})OR^{b}, —C(═NR^{f})NR^{c}R^{d}, —C(═NR^{f})SR^{e}, —C(═O)OR^{b}, —C(═O)NR^{c}R^{d}, —C(═O)SR^{e}, —C(═S)OR^{b}, —C(═S)NR^{c}R^{d}, —OR^{b}, —O—C(═O)R^{a}, —O—C(═O)OR^{b}, —O—C(═O)—NR^{c}R^{d}, —SR^{e}, —S(═O)R^{e}, —S(═O)₂R^{e}, —S(═O)₂NR^{e}R^{d}, —S(═N—R^{a})R^{b}, —S(═O)(═N—R^{a})R^{b}, —NR^{c}R^{d}, —NR^{f}—C(═O)R^{a}, —NR^{f}—C(═O)OR^{b}, —NR^{f}—C(═O)NR^{c}R^{d}, —NR^{f}—NR^{c}R^{d}, —NR^{f}—C(═O)SR^{e} and —NR^{f}—C(═S)NR^{c}R^{d};
wherein R^{a}, R^{a′}, R^{b}, R^{c}, R^{d}, R^{e}, R^{f}, R^{g} and R^{h} are selected independently of one another from the group consisting of hydrogen, C₁-C₆-alkyl, C₂-C₆-alkenyl, C₂-C₆-alkynyl, C₃-C₈-cycloalkyl, saturated or partially unsaturated 5, 6 or 7-membered heterocycles containing 1, 2, 3 or 4 heteroatoms selected independently from one another from O, S, N as ring members, and Ar⁷,
wherein each carbon atom in the aforementioned radicals may carry any combination of 1, 2 or 3 substituents, selected independently of one another from the group consisting of halogen, CN, NO₂, OH, C₁-C₆-alkoxy, C₁-C₆-haloalkoxy, C₁-C₆-alkylthio, C₁-C₆-haloalkylthio, C₁-C₆-alkylcarbonyl, C₁-C₆-haloalkylcarbonyl, C₁-C₆-alkoxycarbonyl, C₁-C₆-haloalkoxycarbonyl, C₁-C₆-alkylamino, di(C₁-C₆-alkyl)amino, C₃-C₆-cycloalkyl, C₃-C₆-halocycloalkyl, Ar⁸, —CH₂—Ar⁸, —O—Ar⁸ and —S—Ar⁸, wherein Ar⁷ and Ar⁸ have one of the meanings given below, and wherein C₃-C₈-cycloalkyl and the heterocycles additionally may carry 1, 2, 3, 4 or 5 substituents selected independently of one another from the group consisting of C₁-C₆-alkyl, C₂-C₆-alkenyl, C₂-C₆-alkynyl and C₁-C₆-haloalkyl; and
Ar¹, Ar², Ar³, Ar⁴, Ar⁵, Ar⁶, Ar⁷, and Ar⁸ are selected independently of one another from the group consisting of phenyl, naphthyl and mono- or bicyclic 5- to 10-membered heteroaryl, which contains 1, 2, 3 or 4 heteroatoms selected from the group consisting of O, S and N as ring members, wherein phenyl, naphtyl and hetaryl are unsubstituted or carry any combination of 1 to 5 substituents, selected independently of one another from the group consisting of halogen, CN, NO₂, OH, C₁-C₆-alkyl, C₂-C₆-alkenyl, C₂-C₆-alkynyl, C₁-C₆-haloalkyl, C₁-C₆-alkoxy, C₁-C₆-haloalkoxy, C₁-C₆-alkylthio, C₁-C₆-haloalkylthio, C₁-C₆-alkylcarbonyl, C₁-C₆-haloalkylcarbonyl, C₁-C₆-alkoxycarbonyl, C₁-C₆-haloalkoxycarbonyl, C₁-C₆-alkylamino, di(C₁-C₆-alkyl)amino, C₃-C₆-cycloalkyl, C₃-C₆-halocycloalkyl, unsubstituted phenyl and phenyl carrying 1, 2, 3, 4 or 5 substituents selected from the group consisting of halogen, C₁-C₆-alkyl, C₁-C₆-haloalkyl, C₁-C₆-alkoxy and C₁-C₆-haloalkoxy;
or the enantiomers, diastereomers or agriculturally or veterinarily acceptable salts thereof.

28. The azolin-2-ylamino compound of claim 27, wherein
n is 1, and
R² and R³ are both hydrogen.

29. The azolin-2-ylamino compound of claim 27, wherein
X is sulfur.

30. The azolin-2-ylamino compound of claim 27, wherein
A is the radical of formulae A.2;
R⁶ is hydrogen; and
R^{7a}, R^{7b}, R^{7c} and R^{7d} are hydrogen.

31. The azolin-2-ylamino compound of claim 27, wherein
Ar is phenyl, which is unsubstituted or carries 1, 2 or 3 radicals R^{Ar}, and wherein
R^{Ar} is selected from the group consisting of halogen, CN, C₁-C₆-alkyl, C₃-C₈-cycloalkyl, formyl, C₁-C₆-alkylcarbonyl, —C(═O)OH, C₁-C₆-alkoxycarbonyl, —C(═O)NH₂, C₁-C₆-alkylaminocarbonyl, di(C₁-C₆-alkyl)aminocarbonyl, —OH, C₁-C₆-alkoxy, —SH, C₁-C₆-alkylthio, —NH₂, C₁-C₆-alkylamino and di(C₁-C₆-alkyl)amino, wherein each carbon atom of the aforementioned radicals may carry any combination of 1, 2 or 3 substituents, selected independently of one another from the group consisting of halogen, CN, NO₂, OH, C₁-C₆-alkoxy, C₁-C₆-haloalkoxy, and C₁-C₆-alkylthio; and/or wherein
two of the radicals R^{Ar} bound to adjacent carbon atoms of the phenyl ring together with said carbon atoms form a fused benzene, a fused saturated or partially unsaturated 5- or 6-membered carbocycle or a fused 5- or 6-membered heterocycle, which contains 1, 2, 3 or 4 heteroatoms selected from the group consisting of O, S and N as ring members, wherein each fused benzene, carbocycle or heterocycle is unsubstituted or carries 1, 2 3 or 4 substituents, selected independently of one another from the group consisting of halogen, CN, NO₂, OH, SH, NH₂, COOH, C₁-C₆-alkyl, C₁-C₆-alkoxy, C₂-C₆-alkenyloxy, C₂-C₆-alkynyloxy, C₁-C₆-haloalkoxy and C₁-C₆-alkylthio.

32. The azolin-2-ylamino compound of claim 27, wherein
R⁴ is selected from the group consisting of hydrogen, halogen, C₁-C₆-alkyl, C₂-C₆-alkenyl, C₂-C₆-alkynyl, C₃-C₈-cycloalkyl, Ar⁵, —C(═O)R^{a}, —C(═S)R^{a}, —C(═O)OR^{b} and —C(═O)NR^{c}R^{d}, wherein
C₁-C₆-alkyl, C₂-C₆-alkenyl, C₂-C₆-alkynyl and C₃-C₈-cycloalkyl are unsubstituted or carry any combination of 1, 2, 3, 4 or 5 substituents, selected independently of one another from the group consisting of halogen, CN, NO₂, OH, C₁-C₆-alkoxy, C₁-C₆-haloalkoxy, C₂-C₆-alkenyloxy, C₂-C₆-alkynyloxy, NH₂, C₁-C₆-alkylamino, di(C₁-C₆-alkyl)amino, —SH, Ar⁶, —CH₂—Ar⁶, —O—Ar⁶ and —S—Ar⁶, and
wherein C₃-C₈-cycloalkyl additionally may carry 1, 2 or 3 substituents selected independently of one another from the group consisting of C₁-C₆-alkyl, C₂-C₆-alkenyl, C₂-C₆-alkynyl and C₁-C₆-haloalkyl.

33. A compound of the general formula (II), 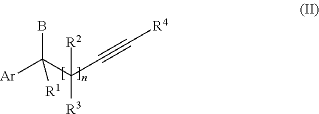
wherein,
n, Ar, R¹, R², R³ and R⁴ have the meanings as defined in claim 27 for the compounds of formula I,
and wherein
B is a radical 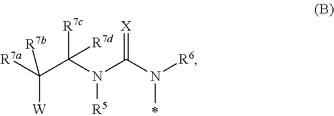
wherein
* indicates the point of attachment to the remaining part of the compound,
R⁵, R⁶, R^{7a}, R^{7b}, R^{7c}, R^{7d} and X have the meanings as defined in claim 27 for the compounds of formula I,
and
W is halogen or —OR⁹, wherein
R⁹ is hydrogen or —C(═O)—Rⁱ, wherein
Rⁱ is selected from the group consisting of hydrogen, C₁-C₆-alkyl, C₂-C₆-alkenyl, C₂-C₆-alkynyl, C₃-C₈-cycloalkyl, saturated or partially unsaturated 5, 6 or 7-membered heterocycles containing 1, 2, 3 or 4 heteroatoms selected independently from one another from the group consisting of O, S and N as ring members, and Ar⁹, wherein
C₁-C₆-alkyl, C₂-C₆-alkenyl, C₂-C₆-alkynyl, C₃-C₈-cycloalkyl and the heterocycles are unsubstituted or carry any combination of 1, 2, 3, 4 or 5 substituents, selected independently of one another from the group consisting of halogen, CN, NO₂, OH, C₁-C₆-alkoxy, C₁-C₆-haloalkoxy, C₁-C₆-alkylthio, C₁-C₆-haloalkylthio, C₁-C₆-alkylcarbonyl, C₁-C₆-haloalkylcarbonyl, C₁-C₆-alkoxycarbonyl, C₁-C₆-haloalkoxycarbonyl, di(C₁-C₆-alkyl)amino, C₃-C₆-cycloalkyl, C₃-C₆-halocycloalkyl, Ar¹⁰, —CH₂—Ar¹⁰, —O—Ar¹⁰ and —S—Ar¹⁰, and wherein
C₃-C₈-cycloalkyl and the heterocycles additionally may carry 1, 2, 3, 4 or 5 substituents selected independently of one another from the group consisting of C₁-C₆-alkyl, C₂-C₆-alkenyl, C₂-C₆-alkynyl and C₁-C₆-haloalkyl;
and wherein
Ar^{g} and Ar¹⁰ are selected independently of one another from the group consisting of phenyl, naphthyl and mono- or bicyclic 5- to 10-membered heteroaryl, which contains 1, 2, 3 or 4 heteroatoms selected from the group consisting of O, S and N as ring members, wherein phenyl, naphtyl and hetaryl are unsubstituted or carry any combination of 1 to 5 substituents, selected independently of one another from the group consisting of halogen, CN, NO₂, OH, C₁-C₆-alkyl, C₂-C₆-alkenyl, C₂-C₆-alkynyl, C₁-C₆-haloalkyl, C₁-C₆-alkoxy, C₁-C₆-haloalkoxy, C₁-C₆-alkylthio, C₁-C₆-haloalkylthio, C₁-C₆-alkylcarbonyl, C₁-C₆-haloalkylcarbonyl, C₁-C₆-alkoxycarbonyl, C₁-C₆-haloalkoxycarbonyl, C₁-C₆-alkylamino, di(C₁-C₆-alkyl)amino, C₃-C₆-cycloalkyl, C₃-C₆-halocycloalkyl, unsubstituted phenyl and phenyl carrying 1, 2, 3, 4 or 5 substituent selected from the group consisting of halogen, C₁-C₆-alkyl, C₁-C₆-haloalkyl, C₁-C₆-alkoxy and C₁-C₆-haloalkoxy,
or the enantiomers, diastereomers or agriculturally or veterinarily acceptable salts thereof.

34. A process for the preparation of the azolin-2-ylamino compound of formula I as defined in claim 27 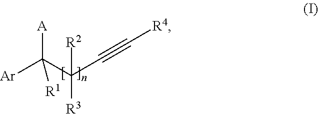
wherein
n, Ar, R¹, R², R³ and R⁴ are as defined in claim 27,
A is a radical of the formulae A.1 or A.2: 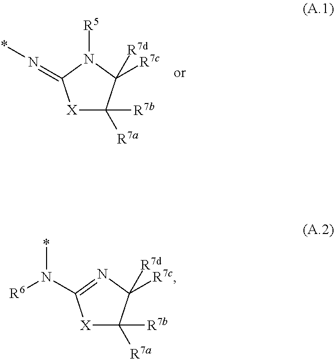
and wherein
X is oxygen or sulfur;
R⁵, R⁶ are hydrogen;
and the other variables and indices are as defined in claim 27;
comprising cyclizing an aminothiocarbonylaminoethanol compound of formula II.A or an aminocarbonylaminoethanol compound of formula II.B, respectively, 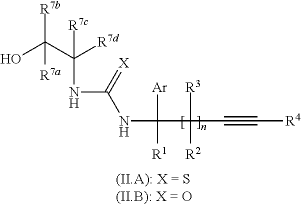
wherein n, R¹, R₂, R³, R⁴, R^{7a}, R^{7b}, R^{7c}, R^{7d} and Ar are as defined in claim 27 for compounds of formula I,
in the presence of an acidic catalyst or under a dehydrating condition.

35. A process for preparing an aminothiocarbonyl-aminoethanol compound of formula II.A or an aminocarbonylaminoethanol compound of formula II.B, 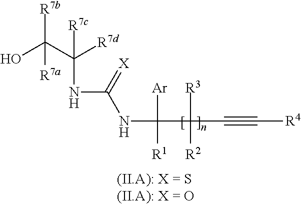
wherein R¹, R², R³, R⁴, R^{7a}, R^{7b}, R^{7c}, R^{7d} and wherein Ar are as defined in claim 33 for compounds of formula II,
comprising converting a compound of formula III 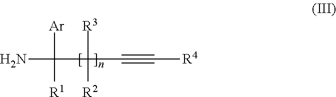
wherein the variables and the index are as defined for compounds of formula II.A and II.B, respectively, or a salt thereof, to the corresponding iso(thio)cyanate IV 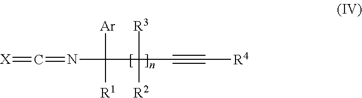
wherein the variables and the index are as defined for compounds of formula II.A and II.B, respectively, with (thio)phosgene, optionally in the presence of a base,
reacting the iso(thio)cyanate IV with an aminoethanol V 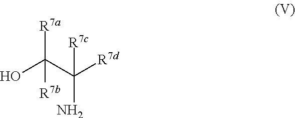
wherein the variables and the index are as defined for compounds of formula II.A and II.B, respectively,
to form compounds II.A or II.B.

36. A process for preparing a compound of formula II.A as defined in claim 34 comprising reacting an amino compound III 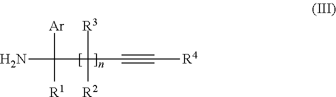
wherein the variables and the index have the meaning as defined for compounds II.A as defined in claim 34, or a salt thereof, with an isothiocyanate VI 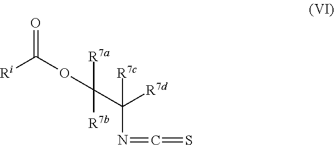
wherein the variables and the index have the meaning as defined for compounds II.A as defined in claim 8 and Rⁱ is as defined in claim 27,
to yield compounds of formula II.A′ 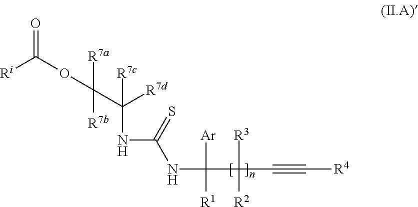
wherein the variables and the index have the meaning as defined for compounds II.A as defined in claim 34, and
followed by saponification of (II.A)′.

37. An agricultural or veterinary composition containing at least one azolin-2-ylamino compound of formula I as defined in claim 27 and/or an enantiomer, diastereomer or agriculturally acceptable salt thereof and at least one liquid or solid carrier.

38. A method for controlling animal pests, said method comprising treating the pests, their food supply, their habitat or their breeding ground or a plant, seed, soil, area, material or environment in which the pests are growing or may grow, or the materials, plants, seeds, soils, surfaces or spaces to be protected from pest attack or infestation with a pesticidally effective amount of the azolin-2-ylamino compound of formula I or an enantiomer, diastereomer or salt thereof as defined in claim 27.

39. A method for controlling animal pests, said method comprising treating the pests, their food supply, their habitat or their breeding ground or a plant, seed, soil, area, material or environment in which the pests are growing or may grow, or the materials, plants, seeds, soils, surfaces or spaces to be protected from pest attack or infestation with a pesticidally effective amount of the agricultural composition of claim 37.

40. A method for protecting plant propagation material or the plants which grow therefrom, said method comprising treating the plant propagation material with a pesticidally effective amount of the azolin-2-ylamino compound of formula I or an enantiomer, diastereomer or agriculturally acceptable salt thereof as defined in claim 27.

41. A method for protecting plant propagation material or the plants which grow therefrom, said method comprising treating the plant propagation material with a pesticidally effective amount of the agricultural composition of claim 37.

42. The method of claim 40, wherein the plant proparagtion material are seeds.

43. A seed treated with at least one azolin-2-ylamino compound of formula I as defined in claim 27 and/or an enantiomer, diastereomer or agriculturally acceptable salt thereof.

44. A method for treating, controlling, preventing or protecting animals against infestation or infection by parasites, said method comprising treating the animals with a parasiticidally effective amount of the azolin-2-ylamino compound of formula I or an enantiomer, diastereomer or veterinally acceptable salt thereof as defined in claim 27.

45. A method for treating, controlling, preventing or protecting animals against infestation or infection by parasites, said method comprising treating the animals with a parasiticidally effective amount of the composition of claim 37.

46. An agricultural or veterinary composition containing at least one compound of formula II as defined in claim 33 and/or an enantiomer, diastereomer or agriculturally acceptable salt thereof and at least one liquid or solid carrier.

47. A method for controlling animal pests, said method comprising treating the pests, their food supply, their habitat or their breeding ground or a plant, seed, soil, area, material or environment in which the pests are growing or may grow, or the materials, plants, seeds, soils, surfaces or spaces to be protected from pest attack or infestation with a pesticidally effective amount of the compound of formula II or an enantiomer, diastereomer or salt thereof as defined in claim 33.

48. A method for protecting plant propagation material and/or the plants which grow therefrom, said method comprising treating the plant propagation material with a pesticidally effective amount of a the compound of formula II or an enantiomer, diastereomer or agriculturally acceptable salt thereof as defined in claim 33.

49. The method of claim 47, wherein the plant propagation material are seeds.

50. A seed treated with at least one compound of formula II as defined in claim 33 and/or an enantiomer, diastereomer or agriculturally acceptable salt thereof.

51. A method for treating, controlling, preventing or protecting animals against infestation or infection by parasites, said method comprising treating the animal with a parasiticidally effective amount of the compound of formula II or an enantiomer, diastereomer or veterinally acceptable salt thereof as defined in claim 33.
